(19) United States
(12) Patent Application Publication
(10) Pub. No.: US 2023/0399618 A1
(43) Pub. Date: Dec. 14, 2023

(54) METHODS, COMPOSITIONS, AND KITS FOR EXPANDING NATURAL KILLER CELLS

(71) Applicant: Regents of the University of Minnesota, Minneapolis, MN (US)

(72) Inventors: Branden Scott Moriarity, Shoreview, MN (US); Jae Woong Chang, Falcon Heights, MN (US); Beau R. Webber, Andover, MN (US)

(73) Assignee: Regents of the University of Minnesota, Minneapolis, MN (US)

(21) Appl. No.: 17/984,720

(22) Filed: Nov. 10, 2022

Related U.S. Application Data
(60) Provisional application No. 63/277,893, filed on Nov. 10, 2021.

(51) Int. Cl. C12N 5/0783; C12N 5/00

(52) U.S. Cl. CPC C12N 5/0646; C12N 5/0018; C12N 2501/2302; C12N 2501/2318; C12N 2501/51; C12N 2501/25; C12N 2501/2321; C12N 2501/599

(57) ABSTRACT

Provided herein are methods, compositions, and kits for expanding natural killer cells in the absence of feeder cells.

### CROSS-REFERENCE TO RELATED APPLICATIONS

This application claims the benefit of priority to U.S. Provisional Patent Application No. 63/277,893, filed on Nov. 10, 2021. The entire content of this application is incorporated herein by reference.

### SEQUENCE LISTING

This application contains a Sequence Listing that has been submitted electronically as an XML file named “52526-0005001_SL_ST26.XML.” The XML file, created on May 16, 2023, is 33,006 bytes in size. The material in the XML file is hereby incorporated by reference in its entirety.

### BACKGROUND

Natural killer (NK) cells are a type of cytotoxic lymphocyte critical to the innate immune system and represent 5-20% of all circulating lymphocytes in humans (Arachchige et al., Innate Immunity 27(3):212-229, 2021). NK cells have analogous functionality to that of cytotoxic T cells in the vertebrate adaptive immune response in that NK cells provide rapid responses to virus-infected cells, other intracellular pathogens, and cancer cells. Typically, immune cells detect the major histocompatibility complex (MHC) presented on infected cell surfaces, triggering cytokine release and causing the death of the infected cell by lysis or apoptosis. NK cells act differently, however, since they can recognize and kill stressed cells in the absence of antibodies and MHC, allowing for a much faster immune response. This function of NK cells is especially important because harmful cells that are missing MHC class I cannot be detected and destroyed by other immune cells, such as T lymphocytes.

Various approaches have been developed to expand large quantities of NK cells for use in clinical applications (e.g., immunotherapy). NK cell expansion requires the delivery of cell signals that promote NK cell survival, proliferation and/or cytotoxicity. To-date, NK cell expansion methods have focused on using autologous feeder cells, genetically modified allogeneic feeder cells, or plasma membrane particles derived from these cells, with limited success. Thus, there remains a need for large-scale feeder-free expansion of NK cells.

### SUMMARY

The present disclosure features in vitro methods, compositions, and kits for expanding natural killer (NK) cells in the absence of feeder cells. The ability to generate clinical doses of NK cells for therapeutic purposes (e.g., chimeric antigen receptor (CAR)-NK cells) is challenging. In general, current methods of expanding natural killer cells include the use of irradiated feeder cells. However, methods including feeder cells are costly, are difficult to scale-up, and can potentially result in the feeder cells, or derivatives thereof, contaminating the expanded NK cell population. Provided herein are methods of expanding NK cells in the absence of feeder cells that can be performed on both a small-scale and a large-scale. The presently provided methods results in an unexpected improvement in the fold-expansion of NK cells (e.g., about 8-fold to about 80-fold expansion of NK cells at about day 10, an additional about 8-fold to about 80-fold expansion of NK cells at about day 20 (e.g., for a total about 64-fold to about 6,400-fold expansion of NK cells at about 20), and a further additional about 8-fold to about 80-fold expansion of NK cells at about day 30 (e.g., for a total about 512-fold to about 512,000-fold expansion of NK cells at about day 30).

Provided herein are methods of expanding an NK cell that include: contacting an NK cell disposed in a liquid culture medium, in the absence of a feeder cell, with (i) a first polypeptide including B7-H6 or a functional fragment thereof, or an anti-NKp30 antigen-binding domain, (ii) a second polypeptide including 4-1BBL or a functional fragment thereof, and (iii) a third polypeptide including ICAM-1 or a functional fragment thereof, under conditions sufficient for expansion of the NK cell.

In some embodiments of any of the methods described herein, after the contacting step the method includes periodically re-contacting the NK cell with the first polypeptide, the second polypeptide, and the third polypeptide. In some embodiments of any of the methods described herein, method is performed over about 30 days, and the re-contacting step is performed at about day 10 and at about day 20 after the contacting step. In some embodiments of any of the methods described herein, the method is performed from about 1 day to about 30 days. In some embodiments of any of the methods described herein, the method is performed from about 5 days to about 30 days. In some embodiments of any of the methods described herein, the method is performed from about 10 days to about 30 days.

In some embodiments of any of the methods described herein, one or more of the first polypeptide, the second polypeptide, and the third polypeptide are present in the liquid culture medium. In some embodiments of any of the methods described herein, two or more of the first polypeptide, the second polypeptide, and the third polypeptide are present in the liquid culture medium. In some embodiments of any of the methods described herein, the first polypeptide, the second polypeptide, and the third polypeptide are present in the liquid culture medium. In some embodiments of any of the methods described herein, one or more of the first polypeptide, the second polypeptide, and the third polypeptide are directly or indirectly attached to one or more substrate(s). In some embodiments of any of the methods described herein, the first polypeptide, the second polypeptide, and the third polypeptide are present in approximately equal amounts or concentrations. In some embodiments of any of the methods described herein, one or more of the first polypeptide, the second polypeptide, and the third polypeptide are directly attached to the one or more substrate(s). In some embodiments of any of the methods described herein, one or more of the first polypeptide, the second polypeptide, and the third polypeptide are indirectly attached to the one or more substrate(s). In some embodiments of any of the methods described herein, one or more of the first polypeptide, the second polypeptide, and the third polypeptide includes a tag. In some embodiments of any of the methods described herein, the one or more substrate(s) includes an agent that specifically binds to the tag. In some embodiments of any of the methods described herein, the tag is an Fc domain, a myc tag, a HA tag, or a GST tag. In some embodiments of any of the methods described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the methods described herein, one or more of the first polypeptide, the second polypeptide, and the third polypeptide further comprises a linker (e.g., an N-terminal or C-terminal linker (e.g., disposed between the substrate and the other portions of the first polypeptide, the second polypeptide, and/or the third polypeptide)). In some embodiments of any of the methods described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the methods described herein, two or more of the first polypeptide, the second polypeptide, and the third polypeptide are directly or indirectly attached to one or more substrate(s). In some embodiments of any of the methods described herein, two or more of the first polypeptide, the second polypeptide, and the third polypeptide are directly attached to the one or more substrate(s). In some embodiments of any of the methods described herein, two or more of the first polypeptide, the second polypeptide, and the third polypeptide are indirectly attached to the one or more substrate(s). In some embodiments of any of the methods described herein, two or more of the first polypeptide, the second polypeptide, and the third polypeptide include a tag. In some embodiments of any of the methods described herein, the one or more substrate(s) includes an agent that specifically binds to the tag (e.g., an anti-Fc antibody or antigen-binding fragment thereof, an anti-myc tag antibody or antigen-binding fragment thereof, an anti-HA tag antibody or antigen-binding fragment thereof, or an anti-GST tag antibody or antigen-binding fragment thereof). In some embodiments of any of the methods described herein, the tag is an Fc domain, a myc tag, a HA tag, or a GST tag. In some embodiments of any of the methods described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the methods described herein, one or more of the first polypeptide, the second polypeptide, and the third polypeptide further comprises a linker (e.g., an N-terminal or C-terminal linker (e.g., disposed between the substrate and the other portions of the first polypeptide, the second polypeptide, and/or the third polypeptide)). In some embodiments of any of the methods described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the methods described herein, first polypeptide, the second polypeptide, and the third polypeptide are directly or indirectly attached to one or more substrate(s). In some embodiments of any of the methods described herein, the first polypeptide, the second polypeptide, and the third polypeptide are directly attached to the one or more substrate(s). In some embodiments of any of the methods described herein, the first polypeptide, the second polypeptide, and the third polypeptide are indirectly attached to the one or more substrate(s). In some embodiments of any of the methods described herein, the first polypeptide, the second polypeptide, and the third polypeptide include a tag. In some embodiments of any of the methods described herein, the one or more substrate(s) includes an agent that specifically binds to the tag (e.g., an anti-Fc antibody or antigen-binding fragment thereof, an anti-myc tag antibody or antigen-binding fragment thereof, an anti-HA tag antibody or antigen-binding fragment thereof, or an anti-GST tag antibody or antigen-binding fragment thereof). In some embodiments of any of the methods described herein, the tag is an Fc domain, a myc tag, a HA tag, or a GST tag. In some embodiments of any of the methods described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the methods described herein, one or more of the first polypeptide, the second polypeptide, and the third polypeptide further comprises a linker (e.g., an N-terminal or C-terminal linker (e.g., disposed between the substrate and the other portions of the first polypeptide, the second polypeptide, and/or the third polypeptide)). In some embodiments of any of the methods described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the methods described herein, the substrate includes a hydrogel. In some embodiments of any of the methods described herein, the substrate includes a natural polymer, a synthetic polymer, or a co-polymer. In some embodiments of any of the methods described herein, the natural polymer is selected from the group consisting of: alginate, agarose, carrageenan, chitosan, dextran, carboxymethylcellulose, heparin, hyaluronic acid, polyamino acids, collagen, gelatin, fibrin, fibrous protein-based biopolymers, and any combination thereof. In some embodiments of any of the methods described herein, the synthetic polymer is selected from the group consisting of: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof. In some embodiments of any of the methods described herein, the co-polymer is a copolymer of natural polymers, a copolymer of synthetic polymers or a copolymer of a natural polymer with a synthetic polymer. In some embodiments of any of the methods described herein, the one or more substrate(s) is a bead. In some embodiments of any of the methods described herein, the bead is a magnetic bead.

In some embodiments of any of the methods described herein, the one or more substrate(s) is a culture vessel. In some embodiments of any of the methods described herein, the culture vessel is a cell culture flask, cell culture bag, tube, plate, dish, or well.

In some embodiments of any of the methods described herein, the liquid culture medium includes a chemically-defined liquid culture medium. In some embodiments of any of the methods described herein, the liquid culture medium is selected from the group consisting of: AIM-V medium, NK MACS medium, EL837 medium, X-VIVO 10 medium, X-VIVO 15 medium, X-VIVO 20 medium, LGM-3 lymphocyte growth medium, and LYMPHOONE T-cell expansion xeno-free medium. In some embodiments of any of the methods described herein, the liquid culture medium is AIM-V medium. In some embodiments of any of the methods described herein, the liquid culture medium includes one or more of: serum, a serum replacement, penicillin, streptomycin, HEPES, and _{L}-alanyl-_{L}-glutamine.

In some embodiments of any of the methods described herein, the liquid culture medium includes one or both of IL-2 or a functional fragment thereof, and IL-18 or a functional fragment thereof. In some embodiments of any of the methods described herein, the liquid culture medium includes about 50 U/mL to about 200 U/mL of IL-2 or a functional fragment thereof. In some embodiments of any of the methods described herein, the liquid culture medium includes about 75 U/mL to about 125 U/mL of IL-2 or a functional fragment thereof. In some embodiments of any of the methods described herein, the liquid culture medium includes about 0.1 nM to about 4.0 nM of IL-18 or a functional fragment thereof. In some embodiments of any of the methods described herein, the liquid culture medium includes about 0.2 nM to about 0.8 nM of IL-18 or a functional fragment thereof. In some embodiments of any of the methods described herein, the liquid culture medium includes an anti-CD16 antibody or antigen-binding fragment thereof. In some embodiments of any of the methods described herein, the liquid culture medium does not include IL-12 or a functional fragment thereof. In some embodiments of any of the methods described herein, the liquid culture medium includes IL-21 or a functional fragment thereof. In some embodiments of any of the methods described herein, the liquid culture medium does not include IL-21 or a functional fragment thereof.

In some embodiments of any of the methods described herein, the method includes replenishing the liquid culture medium one or more times. In some embodiments of any of the methods described herein, the replenishing is performed using fed batch culturing. In some embodiments of any of the methods described herein, the replenishing is performed using perfusion culturing. In some embodiments of any of the methods described herein, the replenishing is performed by replacement of the liquid culture medium. In some embodiments of any of the methods described herein, the replenishing of the liquid culture medium occurs about every one or two days.

In some embodiments of any of the methods described herein, the method includes, before the contacting step, culturing the NK cell in the presence of a feeder cell. In some embodiments of any of the methods described herein, the method includes, after the contacting step, culturing the NK cell in the presence of a feeder cell.

In some embodiments of any of the methods described herein, the method includes, after the contacting step, culturing the NK cell in a liquid culture medium not including each of (i) a first polypeptide including B7-H6 or a functional fragment thereof, or an anti-NKp30 antigen-binding domain, (ii) a second polypeptide including 4-1BBL or a functional fragment thereof, and (iii) a third polypeptide including ICAM-1 or a functional fragment thereof.

In some embodiments of any of the methods described herein, the method includes, before the contacting step, introducing into the NK cell a nucleic acid encoding one or more exogenous polypeptides. In some embodiments of any of the methods described herein, the method includes, after the contacting step, introducing into the NK cell a nucleic acid encoding one or more exogenous polypeptides. In some embodiments of any of the methods described herein, the one or more exogenous polypeptides is a chimeric antigen receptor or a therapeutic protein. In some embodiments of any of the methods described herein, the NK cell is a genetically-engineered NK cell.

In some embodiments of any of the methods described herein, the method results in about 8-fold to about 512,000-fold expansion of NK cells. In some embodiments of any of the methods described herein, the method results in about 8-fold to about 80-fold expansion of NK cells at about day 10 after the contacting step. In some embodiments of any of the methods described herein, the method results in about 64-fold to about 6,400-fold expansion of NK cells at about day 20 after the contacting step. In some embodiments of any of the methods described herein, the method results in about 512-fold to about 512,000-fold expansion of NK cells at about day 30 after the contacting step.

Also provided herein are methods of expanding an NK cell including: contacting an NK cell disposed in a liquid culture medium, in the absence of a feeder cell, with (i) a first polypeptide including B7-H6 or a functional fragment thereof, or an anti-NKp30 antigen-binding domain, (ii) a second polypeptide including 4-1BBL or a functional fragment thereof, (iii) a third polypeptide including ICAM-1 or a functional fragment thereof, and (iv) a fourth polypeptide including IL-21 or a functional fragment thereof, under conditions sufficient for expansion of the NK cell.

In some embodiments of any of the methods described herein, after the contacting step the method includes periodically re-contacting the NK cell with the first polypeptide, the second polypeptide, the third polypeptide, and the fourth polypeptide. In some embodiments of any of the methods described herein, the method is performed over about 30 days and the re-contacting step is performed at about day 10 and at about day 20 after the contacting step. In some embodiments of any of the methods described herein, the method is performed over about 1 day to about 30 days. In some embodiments of any of the methods described herein, the method is performed over about 5 days to about 30 days. In some embodiments of any of the methods described herein, the method is performed over about 10 days to about 30 days.

In some embodiments of any of the methods described herein, one or more of the first polypeptide, the second polypeptide, the third polypeptide, and the fourth polypeptide are present in the liquid culture medium. In some embodiments of any of the methods described herein, two or more of the first polypeptide, the second polypeptide, the third polypeptide, and the fourth polypeptide are present in the liquid culture medium. In some embodiments of any of the methods described herein, three or more of the first polypeptide, the second polypeptide, the third polypeptide, and the fourth polypeptide are present in the liquid culture medium. In some embodiments of any of the methods described herein, the first polypeptide, the second polypeptide, the third polypeptide, and the fourth polypeptide are present in the liquid culture medium.

In some embodiments of any of the methods described herein, one or more of the first polypeptide, the second polypeptide, the third polypeptide, and the fourth polypeptide are directly or indirectly attached to one or more substrate(s). In some embodiments of any of the methods described herein, the first polypeptide, the second polypeptide, the third polypeptide, and the fourth polypeptide are present in approximately equal amounts or concentrations. In some embodiments of any of the methods described herein, one or more of the first polypeptide, the second polypeptide, the third polypeptide, and the fourth polypeptide are directly attached to the one or more substrate(s). In some embodiments of any of the methods described herein, one or more of the first polypeptide, the second polypeptide, the third polypeptide, and the fourth polypeptide are indirectly attached to the one or more substrate(s). In some embodiments of any of the methods described herein, one or more of the first polypeptide, the second polypeptide, the third polypeptide, and the fourth polypeptide includes a tag. In some embodiments of any of the methods described herein, the one or more substrate(s) includes an agent that specifically binds to the tag. In some embodiments of any of the methods described herein, the tag is an Fc domain, a myc tag, a HA tag, or a GST tag. In some embodiments of any of the methods described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the methods described herein, one or more of the first polypeptide, the second polypeptide, the third polypeptide, and the fourth polypeptide further comprises a linker (e.g., an N-terminal or C-terminal linker (e.g., disposed between the substrate and the other portions of the first polypeptide, the second polypeptide, the third polypeptide, and/or the fourth polypeptide)). In some embodiments of any of the methods described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the methods described herein, two or more of the first polypeptide, the second polypeptide, the third polypeptide, and the fourth polypeptide are directly or indirectly attached to one or more substrate(s). In some embodiments of any of the methods described herein, two or more of the first polypeptide, the second polypeptide, the third polypeptide, and the fourth polypeptide are directly attached to the one or more substrate(s). In some embodiments of any of the methods described herein, two or more of the first polypeptide, the second polypeptide, the third polypeptide, and the fourth polypeptide are indirectly attached to the one or more substrate(s). In some embodiments of any of the methods described herein, two or more of the first polypeptide, the second polypeptide, the third polypeptide, and the fourth polypeptide include a tag. In some embodiments of any of the methods described herein, the one or more substrate(s) includes an agent that specifically binds to the tag. In some embodiments of any of the methods described herein, the tag is an Fc domain, a myc tag, a HA tag, or a GST tag. In some embodiments of any of the methods described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the methods described herein, one or more of the first polypeptide, the second polypeptide, the third polypeptide, and the fourth polypeptide further comprises a linker (e.g., an N-terminal or C-terminal linker (e.g., disposed between the substrate and the other portions of the first polypeptide, the second polypeptide, the third polypeptide, and/or the fourth polypeptide)). In some embodiments of any of the methods described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the methods described herein, three or more of the first polypeptide, the second polypeptide, the third polypeptide, and the fourth polypeptide are directly or indirectly attached to one or more substrate(s). In some embodiments of any of the methods described herein, three or more of the first polypeptide, the second polypeptide, the third polypeptide, and the fourth polypeptide are directly attached to the one or more substrate(s). In some embodiments of any of the methods described herein, three or more of the first polypeptide, the second polypeptide, the third polypeptide, and the fourth polypeptide are indirectly attached to the one or more substrate(s). In some embodiments of any of the methods described herein, three or more of the first polypeptide, the second polypeptide, the third polypeptide, and the fourth polypeptide include a tag. In some embodiments of any of the methods described herein, the one or more substrate(s) includes an agent that specifically binds to the tag. In some embodiments of any of the methods described herein, the tag is an Fc domain, a myc tag, a HA tag, or a GST tag. In some embodiments of any of the methods described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the methods described herein, one or more of the first polypeptide, the second polypeptide, the third polypeptide, and the fourth polypeptide further comprises a linker (e.g., an N-terminal or C-terminal linker (e.g., disposed between the substrate and the other portions of the first polypeptide, the second polypeptide, the third polypeptide, and/or the fourth polypeptide)). In some embodiments of any of the methods described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the methods described herein, the first polypeptide, the second polypeptide, the third polypeptide, and the fourth polypeptide are directly or indirectly attached to one or more substrate(s). In some embodiments of any of the methods described herein, the first polypeptide, the second polypeptide, the third polypeptide, and the fourth polypeptide are directly attached to the one or more substrate(s). In some embodiments of any of the methods described herein, the first polypeptide, the second polypeptide, the third polypeptide, and the fourth polypeptide are indirectly attached to the one or more substrate(s). In some embodiments of any of the methods described herein, the first polypeptide, the second polypeptide, the third polypeptide, and the fourth polypeptide include a tag. In some embodiments of any of the methods described herein, the one or more substrate(s) includes an agent that specifically binds to the tag. In some embodiments of any of the methods described herein, the tag is an Fc domain, a myc tag, a HA tag, or a GST tag. In some embodiments of any of the methods described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the methods described herein, one or more of the first polypeptide, the second polypeptide, the third polypeptide, and the fourth polypeptide further comprises a linker (e.g., an N-terminal or C-terminal linker (e.g., disposed between the substrate and the other portions of the first polypeptide, the second polypeptide, the third polypeptide, and/or the fourth polypeptide)). In some embodiments of any of the methods described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the methods described herein, the substrate includes a hydrogel. In some embodiments of any of the methods described herein, the substrate includes a natural polymer, a synthetic polymer, or a co-polymer. In some embodiments of any of the methods described herein, the natural polymer is selected from the group consisting of: alginate, agarose, carrageenan, chitosan, dextran, carboxymethylcellulose, heparin, hyaluronic acid, polyamino acids, collagen, gelatin, fibrin, fibrous protein-based biopolymers, and any combination thereof. In some embodiments of any of the methods described herein, the synthetic polymer is selected from the group consisting of: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof. In some embodiments of any of the methods described herein, the co-polymer is a copolymer of natural polymers, a copolymer of synthetic polymers or a copolymer of a natural polymer with a synthetic polymer. In some embodiments of any of the methods described herein, the one or more substrate(s) is a bead. In some embodiments of any of the methods described herein, the bead is a magnetic bead.

In some embodiments of any of the methods described herein, the one or more substrate(s) is a culture vessel. In some embodiments of any of the methods described herein, the culture vessel is a cell culture flask, cell culture bag, tube, plate, dish, or well.

In some embodiments of any of the methods described herein, the liquid culture medium is a chemically-defined liquid culture medium. In some embodiments of any of the methods described herein, the liquid culture medium is selected from the group consisting of: AIM-V medium, NK MACS medium, EL837 medium, X-VIVO 10 medium, X-VIVO 15 medium, X-VIVO 20 medium, LGM-3 lymphocyte growth medium, and LYMPHOONE T-cell expansion xeno-free medium. In some embodiments of any of the methods described herein, the liquid culture medium is AIM-V medium. In some embodiments of any of the methods described herein, the liquid culture medium includes one or more of: serum, a serum replacement, penicillin, streptomycin, HEPES, and _{L}-alanyl-_{L}-glutamine.

In some embodiments of any of the methods described herein, the liquid culture medium includes one or both of IL-2 or a functional fragment thereof, and IL-18 or a functional fragment thereof. In some embodiments of any of the methods described herein, the liquid culture medium includes about 50 U/mL to about 200 U/mL of IL-2 or a functional fragment thereof. In some embodiments of any of the methods described herein, the liquid culture medium includes about 75 U/mL to about 125 U/mL of IL-2 or a functional fragment thereof. In some embodiments of any of the methods described herein, the liquid culture medium includes about 0.1 nM to about 4.0 nM of IL-18 or a functional fragment thereof. In some embodiments of any of the methods described herein, the liquid culture medium includes about 0.2 nM to about 0.8 nM of IL-18 or a functional fragment thereof. In some embodiments of any of the methods described herein, the liquid culture medium includes an anti-CD16 antibody or antigen-binding fragment thereof. In some embodiments of any of the methods described herein, the liquid culture medium does not include IL-12 or a functional fragment thereof.

In some embodiments of any of the methods described herein, the method includes replenishing the liquid culture medium one or more times. In some embodiments of any of the methods described herein, the replenishing is performed using fed batch culturing. In some embodiments of any of the methods described herein, the replenishing is performed using perfusion culturing. In some embodiments of any of the methods described herein, the replenishing is performed by replacement of the liquid culture medium. In some embodiments of any of the methods described herein, the replenishing of the liquid culture medium occurs about every one or two days.

In some embodiments of any of the methods described herein, the method includes, before the contacting step, culturing the NK cell in the presence of a feeder cell. In some embodiments of any of the methods described herein, the method includes, after the contacting step, culturing the NK cell in the presence of a feeder cell. In some embodiments of any of the methods described herein, the method includes, after the contacting step, culturing the NK cell in a liquid culture medium not including each of (i) a first polypeptide including B7-H6 or a functional fragment thereof, or an anti-NKp30 antigen-binding domain, (ii) a second polypeptide including 4-1BBL or a functional fragment thereof, (iii) a third polypeptide including ICAM-1 or a functional fragment thereof; and (iv) a fourth polypeptide including IL-21 or a functional fragment thereof.

In some embodiments of any of the methods described herein, the method includes, before the contacting step, introducing into the NK cell a nucleic acid encoding one or more exogenous polypeptides. In some embodiments of any of the methods described herein, the method includes, after the contacting step, introducing into the NK cell a nucleic acid encoding one or more exogenous polypeptides. In some embodiments of any of the methods described herein, the one or more exogenous polypeptides is a chimeric antigen receptor or a therapeutic protein. In some embodiments of any of the methods described herein, the NK cell is a genetically engineered NK cell.

In some embodiments of any of the methods described herein, the method results in about 8-fold to about 512,000-fold expansion of NK cells. In some embodiments of any of the methods described herein, the method results in about 8-fold to about 80-fold expansion of NK cells at about day 10 after the contacting step. In some embodiments of any of the methods described herein, the method results in about 64-fold to about 6,400-fold expansion of NK cells at about day 20 after the contacting step. In some embodiments of any of the methods described herein, the method results in about 512-fold to about 512,000-fold expansion of NK cells at about day 30 after the contacting step.

Also provided herein are NK cells produced by any of the methods described herein.

Also provided herein are pharmaceutical compositions including any of the NK cells described herein.

Also provided herein are methods of treating a subject in need thereof including administering to the subject any of the NK cells described herein or any of the pharmaceutical composition described herein.

Also provided herein are kits that include: one or more substrate(s) that together include: (i) a first polypeptide including B7-H6 or a functional fragment thereof, or an anti-NKp30 antigen-binding domain, (ii) a second polypeptide including 4-1BBL or a functional fragment thereof, and (iii) a third polypeptide including ICAM-1 or a functional fragment thereof, directly or indirectly attached to the one or more substrate(s). In some embodiments of any of the kits described herein, the first polypeptide, the second polypeptide, and the third polypeptide are directly attached to the one or more substrate(s). In some embodiments of any of the kits described herein, the first polypeptide, the second polypeptide, and the third polypeptide are indirectly attached to the one or more substrate(s). In some embodiments of any of the kits described herein, the first polypeptide, the second polypeptide, and the third polypeptide include a tag.

In some embodiments of any of the kits described herein, the one or more substrate(s) includes an agent that specifically binds to the tag. In some embodiments of any of the kits described herein, the tag is an Fc domain, a myc tag, a HA tag, or a GST tag. In some embodiments of any of the kits described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the kits described herein, one or more of the first polypeptide, the second polypeptide, and the third polypeptide further comprises a linker. In some embodiments of any of the kits described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the kits described herein, the first polypeptide, the second polypeptide, and the third polypeptide are present in approximately equal amounts or concentrations.

In some embodiments of any of the kits described herein, the one or more substrate(s) includes a hydrogel. In some embodiments of any of the kits described herein, the one or more substrate(s) includes a natural polymer, a synthetic polymer, or a co-polymer. In some embodiments of any of the kits described herein, the natural polymer is selected from the group consisting of: alginate, agarose, carrageenan, chitosan, dextran, carboxymethylcellulose, heparin, hyaluronic acid, polyamino acids, collagen, gelatin, fibrin, fibrous protein-based biopolymers, and any combination thereof. In some embodiments of any of the kits described herein, the synthetic polymer is selected from the group consisting of: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof. In some embodiments of any of the kits described herein, the co-polymer is a copolymer of natural polymers, a copolymer of synthetic polymers or a copolymer of a natural polymer with a synthetic polymer. In some embodiments of any of the kits described herein, the one or more substrate(s) is a bead. In some embodiments of any of the kits described herein, the bead is a magnetic bead.

In some embodiments of any of the kits described herein, the one or more substrate(s) is a culture vessel. In some embodiments of any of the kits described herein, the culture vessel is a cell culture flask, cell culture bag, tube, plate, dish, or well.

In some embodiments of any of the kits described herein, the kit includes a liquid culture medium. In some embodiments of any of the kits described herein, the liquid culture medium is a chemically-defined liquid culture medium. In some embodiments of any of the kits described herein, the liquid culture medium is selected from the group consisting of: AIM-V medium, NK MACS medium, EL837 medium, X-VIVO 10 medium, X-VIVO 15 medium, X-VIVO 20 medium, LGM-3 lymphocyte growth medium, and LYMPHOONE T-cell expansion xeno-free medium. In some embodiments of any of the kits described herein, the liquid culture medium is AIM-V medium. In some embodiments of any of the kits described herein, the liquid culture medium includes one or more of: serum, a serum replacement, penicillin, streptomycin, HEPES, and _{L}-alanyl-_{L}-glutamine. In some embodiments of any of the kits described herein, the liquid culture medium includes one or both of IL-2 or a functional fragment thereof and IL-18 or a functional fragment thereof. In some embodiments of any of the kits described herein, the liquid culture medium includes about 50 U/mL to about 200 U/mL of IL-2 or a functional fragment thereof. In some embodiments of any of the kits described herein, the liquid culture medium includes about 75 U/mL to about 125 U/mL of IL-2 or a functional fragment thereof. In some embodiments of any of the kits described herein, the liquid culture medium includes about 0.1 nM to about 4.0 nM of IL-18 or a functional fragment thereof. In some embodiments of any of the kits described herein, the liquid culture medium includes about 0.2 nM to about 0.8 nM of IL-18 or a functional fragment thereof. In some embodiments of any of the kits described herein, the liquid culture medium includes an anti-CD16 antibody or antigen-binding fragment thereof. In some embodiments of any of the kits described herein, the liquid culture medium does not include IL-12 or a functional fragment thereof. In some embodiments of any of the kits described herein, the liquid culture medium includes IL-21 or a functional fragment thereof. In some embodiments of any of the kits described herein, the liquid culture medium does not include IL-21 or a functional fragment thereof.

Also provided herein are kits that include: one or more substrate(s) that together include: (i) a first polypeptide including B7-H6 or a functional fragment thereof, or an anti-NKp30 antigen-binding domain, (ii) a second polypeptide including 4-1BBL or a functional fragment thereof, (iii) a third polypeptide including ICAM-1 or a functional fragment thereof, and (iv) a fourth polypeptide including IL-21 or a functional fragment thereof, directly or indirectly attached to the one or more substrate(s).

In some embodiments of any of the kits described herein, the first polypeptide, the second polypeptide, the third polypeptide, and the fourth polypeptide are directly attached to the one or more substrate(s). In some embodiments of any of the kits described herein, the first polypeptide, the second polypeptide, the third polypeptide, and the fourth polypeptide are indirectly attached to the one or more substrate(s). In some embodiments of any of the kits described herein, the first polypeptide, the second polypeptide, the third polypeptide, and the fourth polypeptide include a tag. In some embodiments of any of the kits described herein, the one or more substrate(s) include(s) an agent that specifically binds to the tag. In some embodiments of any of the kits described herein, the tag is an Fc domain, a myc tag, a HA tag, or a GST tag. In some embodiments of any of the kits described herein, the tag is an Fc domain, and the agent that specifically binds to the tag is an anti-Fc antibody or antigen-binding fragment thereof; the tag is a myc tag and the agent is an anti-myc tag antibody or antigen-binding fragment thereof; the tag is an HA tag and the agent that specifically binds to the tag is an anti-HA tag antibody or antigen-binding fragment thereof; or the tag is a GST tag and the agent that specifically binds to the tag is an anti-GST tag antibody or antigen-binding fragment thereof. In some embodiments of any of the kits described herein, one or more of the first polypeptide, the second polypeptide, the third polypeptide, and the fourth polypeptide further comprises a linker. In some embodiments of any of the kits described herein, the linker comprises a glycine-serine (GS) repeat and/or an IgG hinge region (e.g., an IgG1 hinge region, an IgG2 hinge region, and IgG3 hinge region, or an IgG4 hinge region).

In some embodiments of any of the kits described herein, the first polypeptide, the second polypeptide, the third polypeptide, and the fourth polypeptide are present in approximately equal amounts or concentrations.

In some embodiments of any of the kits described herein, the one or more substrate(s) includes a hydrogel. In some embodiments of any of the kits described herein, the one or more substrate(s) includes a natural polymer, a synthetic polymer, or a co-polymer. In some embodiments of any of the kits described herein, the natural polymer is selected from the group consisting of: alginate, agarose, carrageenan, chitosan, dextran, carboxymethylcellulose, heparin, hyaluronic acid, polyamino acids, collagen, gelatin, fibrin, fibrous protein-based biopolymers, and any combination thereof. In some embodiments of any of the kits described herein, the synthetic polymer is selected from the group consisting of: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof. In some embodiments of any of the kits described herein, the co-polymer is a copolymer of natural polymers, a copolymer of synthetic polymers or a copolymer of a natural polymer with a synthetic polymer. In some embodiments of any of the kits described herein, the one or more substrate(s) is a bead. In some embodiments of any of the kits described herein, the bead is a magnetic bead.

In some embodiments of any of the kits described herein, the one or more substrate(s) is a culture vessel. In some embodiments of any of the kits described herein, the culture vessel is a cell culture flask, cell culture bag, tube, plate, dish, or well.

In some embodiments of any of the kits described herein, the kit includes a liquid culture medium. In some embodiments of any of the kits described herein, the liquid culture medium is a chemically-defined liquid culture medium. In some embodiments of any of the kits described herein, the liquid culture medium is selected from the group consisting of: AIM-V medium, NK MACS medium, EL837 medium, X-VIVO 10 medium, X-VIVO 15 medium, X-VIVO 20 medium, LGM-3 lymphocyte growth medium, and LYMPHOONE T-cell expansion xeno-free medium. In some embodiments of any of the kits described herein, the liquid culture medium is AIM-V medium. In some embodiments of any of the kits described herein, the liquid culture medium includes one or more of: serum, a serum replacement, penicillin, streptomycin, HEPES, and _{L}-alanyl-_{L}-glutamine.

In some embodiments of any of the kits described herein, the liquid culture medium includes one or both of IL-2 or a functional fragment thereof, and IL-18 or a functional fragment thereof. In some embodiments of any of the kits described herein, the liquid culture medium includes about 50 U/mL to about 200 U/mL of IL-2 or a functional fragment thereof. In some embodiments of any of the kits described herein, the liquid culture medium includes about 75 U/mL to about 125 U/mL of IL-2 or a functional fragment thereof. In some embodiments of any of the kits described herein, the liquid culture medium includes about 0.1 nM to about 4.0 nM of IL-18 or a functional fragment thereof. In some embodiments of any of the kits described herein, the liquid culture medium includes about 0.2 nM to about 0.8 nM of IL-18 or a functional fragment thereof. In some embodiments of any of the kits described herein, the liquid culture medium includes an anti-CD16 antibody or antigen-binding fragment thereof. In some embodiments of any of the kits described herein, the liquid culture medium does not include IL-12 or a functional fragment thereof.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The term “perfusion culturing” means a culturing method that includes both the addition (incremental or continuous) of a liquid culture medium to a cell culture and removal of liquid culture medium from the cell culture. Removal and addition can be performed simultaneously or sequentially, or a combination of the two. Further, removal and addition can be performed continuously.

The volume of the liquid culture medium removed and the volume of the liquid culture medium added can in some instances be held approximately the same over each 24-hour period (or, alternatively, an incremental time period of about 1 hour to about 24 hours or an incremental time period of greater than 24 hours) over the entire or part of the culturing period. As is known in the art, the rate at which the volume of the liquid culture medium is removed (volume/unit of time) and the rate at which the volume of the liquid culture medium is added (volume/unit of time) can be varied. The rate at which the volume of the liquid culture medium is removed (volume/unit of time) and the rate at which the volume of the liquid culture medium is added (volume/unit of time) can be about the same or can be different.

Alternatively, the volume removed and added can change (e.g., gradually increase) over each 24-hour period (or alternatively, an incremental time period of between 1 hour and about 24 hours or an incremental time period of greater than 24 hours) during the culturing period. For example the volume of the liquid culture medium removed and the volume of the liquid culture medium added within each 24-hour period (or alternatively, an incremental time period of between about 1 hour and above 24 hours or an incremental time period of greater than 24 hours) over the culturing period can be increased (e.g., gradually or through staggered increments) over the culturing period.

The volume of the liquid culture medium can be removed, e.g., by a mechanical system that can remove the volume of the liquid culture medium from the vessel (e.g., a bioreactor), by allowing the cells to settle and removing the volume of the liquid culture medium using pipetting, or by a method that can at least partially include the use of centrifugal force). Alternatively, or in addition, the volume of the liquid culture medium can be removed by seeping or gravity flow of the volume of the liquid culture medium through a sterile membrane with a molecular weight cut-off (e.g., that excludes NK cells).

The volume of the liquid culture medium can be added to the vessel (e.g., bioreactor) in an automated fashion, e.g., by a peristaltic pump or a perfusion pump.

The term “feed-batch culture” means a culturing method that includes the incremental or continuous addition of a second liquid culture medium to an initial cell culture without substantial or significant removal of the first liquid culture medium from the cell culture. In some instances, the second liquid culture medium includes the same components at substantially the same concentration as the first liquid culture medium. In other instances, the second liquid culture medium is a concentrated form of the first liquid culture medium and/or is added as a dry powder.

The term “chemically-defined liquid culture medium” means a liquid culture medium in which all of the chemical components are known. For example, a chemically-defined liquid culture medium does not contain serum.

The term “functional fragment” means a portion of an amino acid sequence (e.g., of a polypeptide) that is substantially identical to, but shorter in length than, a reference polypeptide that retains at least 80%, at least 90%, at least 95%, at least 96%, at least 97%, at least 98%, or at least 99% of one activity (e.g., cognate binding partner (e.g., receptor) binding activity) of the reference polypeptide. A fragment can include an N-terminal truncation, a C-terminal truncation, or both N-terminal and C-terminal truncations relative to a reference polypeptide.

The terms “polypeptide” and “protein” are used interchangeably herein and refer to a polymer of amino acid residues.

The term “subject” refers to any mammal. In some embodiments, the subject or “subject in need” of a treatment can be a primate (e.g., a human, a simian (e.g., a monkey (e.g., marmoset or baboon), or an ape (e.g., a gorilla, chimpanzee, orangutan, or gibbon)), a rodent (e.g., a mouse, a guinea pig, a hamster, or a rat), a rabbit, a dog, a cat, a horse, a sheep, a cow, a pig, or a goat. In some embodiments, the subject or “subject suitable for treatment” may be a non-human mammal, especially mammals that are conventionally used as models for demonstrating therapeutic efficacy in humans (e.g., a mouse, a pig, a rat, or a non-human primate) may be employed. In some examples, a subject can be previously diagnosed or identified as being in need of treatment by a medical professional (e.g., a physician, a laboratory technician, a physician's assistant, a nurse, or a clinical laboratory technician).

As used herein, “treating” means a reduction in the number, severity, frequency, and/or duration of one or more symptoms of a medical disease or condition in a subject (e.g., any of the exemplary subjects described herein).

The term “flask” means a vessel (e.g., a sterile vessel) that can hold a volume of liquid culture medium that has at least one gas permeable surface (e.g., an end that has a gas-permeable element, e.g., a membrane, which may also act as a sterile barrier) and/or at least one vent cap, and at least a portion of its shape is approximately frustoconical. For example, a flask can be a cell culture flask, such as a T-flask, an Erlenmeyer flask, or any art-recognized modified version thereof.

The term “tube” means a vessel (e.g., a sterile vessel) that can retain liquid culture medium that has at least one gas permeable surface (e.g., an end that has a gas-permeable element, e.g., a membrane, which may also act as a sterile barrier) and/or at least one vent cap, and is capable of retaining liquid culture medium within the vessel upon agitation (e.g., rotary agitation), and at least a portion of its shape is approximately cylindrical. For example, a tube can be an Eppendorf tube (e.g., a 50-mL or 15-mL Eppendorf tube), a roller bottle, or any art-recognized equivalent or modified version thereof.

### BRIEF DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIGS. 1A-1G are graphs comparing a feeder-free NK cell expansion method and feeder cell-dependent NK cell expansion method at different time points using NK cells from two separate donors. FIG. 1A is a bar graph showing the fold expansion of NK cells at day 10 for a feeder-free NK cell expansion method (diamonds) and a feeder cell-dependent NK cell expansion method (inverted triangles). FIG. 1B is a bar graph showing the fold expansion of NK cells between day 10 and day 20 for a feeder-free NK cell expansion method (diamonds) and a feeder cell-dependent NK cell expansion method (inverted triangles). FIG. 1C is a bar graph showing the fold expansion of NK cells between day 0 and day 20 for a feeder-free NK cell expansion method (diamonds) and a feeder cell-dependent NK cell expansion method (inverted triangles). FIG. 1D is a line graph showing the total number of NK cells for a feeder-free NK cell expansion method (squares) and a feeder cell-dependent NK cell expansion method (circles) at days 0, 10 and 20. FIG. 1E is a bar graph showing the fold expansion of NK cells between day 20 and day 30 for a feeder-free NK cell expansion method (diamonds) and a feeder cell-dependent NK cell expansion method (inverted triangles). FIG. 1F is a bar graph showing the fold expansion of NK cells between day 0 and day 30 for a feeder-free NK cell expansion method (diamonds) and a feeder cell-dependent NK cell expansion method (inverted triangles). FIG. 1G is a line graph showing the total number of NK cells for a feeder-free NK cell expansion method (squares) and a feeder cell-dependent NK cell expansion method (circles) at days 0, 10, 20 and 30.

FIGS. 2A-2D are bar graphs comparing the cytolytic activity against different cell lines of feeder-free expanded NK cells with NK cells expanded using feeder cell-expanded NK cells. FIG. 2A is a bar graph showing the percent of cell lysis of K562 cells after 24 hours with either feeder-free expanded NK cells (circles) or feeder cell-expanded NK cells (squares). FIG. 2B is a bar graph showing the percent of cell lysis of Raji cells after 24 hours with either feeder-free expanded NK cells (circles) or feeder cell-expanded NK cells (squares). FIG. 2C is a bar graph showing the percent of cell lysis of Nomo-1 cells after 24 hours with either feeder-free expanded NK cells (circles) or feeder cell-expanded NK cells (squares). FIG. 2D is a bar graph showing the percent of cell lysis of Molm-13 cells after 24 hours with either feeder-free expanded NK cells (circles) or feeder cell-expanded NK cells (squares).

FIG. 3 is a bar graph comparing telomere length (kb/chromosome) in NK cells prior to expansion at Day 0 (inverted triangle), feeder cell-expanded expanded NK cells (diamonds) and feeder-free expanded NK cells (empty circle).

### DETAILED DESCRIPTION

Natural killer (NK) cells are innate lymphocytes important for mediating anti-viral and anti-cancer immunity through cytokine and chemokine secretion, and through the release of cytotoxic granules (see, e.g., Vivier et al. Science 331(6013):44-49, 2011; Caligiuri, Blood 112(3):461-469, 2008; and Roda et al., Cancer Res. 66(1):517-526, 2006). NK cells are effector cells that include the third largest population of lymphocytes and are important for host immune-surveillance against tumor and pathogen-infected cells.

Expanding NK cells is critical to achieve quantities sufficient for clinical applications. Expansion protocols using feeder cells (e.g., irradiated peripheral blood mononuclear cells or cancer cell lines (e.g., K562 cells genetically modified to express one or more cytokines)) have been described. However, the use of feeder cells imposes complexities to cell processing and analysis. Moreover, NK cell expansion using feeder cells may result in unwanted NK cell uptake and acquisition of membrane-bound proteins (e.g., engineered cytokines) present on the feeder cells via a process known as trogocytosis (see, e.g., Somanchi et al., Blood 119:5164-72, 2012). Such acquisition may present potential clinical and regulatory concerns for expanded NK cells in therapeutic applications. While some feeder-free NK expansion methods with limited cell expansion have also been described, there remains a need for feeder free methods that result in robust NK cell expansion

The methods described herein can be used for small-scale and large-scale expansion of NK cells (e.g., for use in cell therapy). In addition, the specific combinations of polypeptides described herein can be adapted for inclusion in research grade products (e.g., magnetic bead, hydrogel-based systems, or polymer-based systems). For instance, one or more polypeptides from the described specific combination of polypeptides can be complexed (e.g., conjugated) with a substrate(s) such as beads and other cell culture apparatuses to generate kits for in vitro NK cell expansion.

In some embodiments, the methods described herein do not require the use of antigen-binding proteins (e.g., antibodies) which can be advantageous during cell therapy manufacturing as it reduces the risk of residual antibodies in the manufactured drug substance. In contrast, many of the feeder-free NK cell expansion methods known in the art require at least one antibody (e.g., an anti-NKp46 antibody). In some embodiments, the methods provided herein do not comprise contacting the NK cells with an anti-CD2 antibody or antigen-binding fragment thereof. In some embodiments, the methods provided herein do not comprise contacting the NK cells with an anti-NKp46 antibody or antigen-binding fragment thereof. In some embodiments, the methods provided herein do not comprise contacting the NK cells with an anti-CD2 antibody or antigen-binding fragment thereof and an anti-NKp46 antibody or antigen-binding fragment thereof.

The methods provided herein also provide for an unexpectedly improved level of expansion as compared to known NK cell expansion methods (e.g., known feeder-free NK cell expansion methods). In some embodiments, following the contacting (e.g., an initial contacting step) (e.g., for about 1 hour, 2 hours, 3 hours, 4 hours, 5 hours, 6 hours, 7 hours, 8 hours, 9 hours, hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 4 days, 5 days, 6 days, 7 days, 8 days, 9 days, 10 days, 11 days, or 12 days) of the NK cell with the first polypeptide, the second polypeptide, and the third polypeptide, and optionally, the fourth polypeptide, under conditions sufficient for expansion of the NK cell, the methods provided herein provide for about 8-fold to about 80-fold (e.g., about 8-fold to about 75-fold, about an 8-fold to 70-fold, about an 8-fold to about 65-fold, about an 8-fold to about 60-fold, about an 8-fold to about 55-fold, about an 8-fold to about 50-fold, about 8-fold to about 45-fold, about 8-fold to about 40-fold, about 8-fold to about 35-fold, about 8-fold to about 30-fold, about 8-fold to about 25-fold, about 8-fold to about 20-fold, about 8-fold to about 15-fold, about 8-fold to about about 10-fold to about 80-fold, about 10-fold to about 75-fold, about 10-fold to about about 10-fold to about 65-fold, about 10-fold to about 60-fold, about 10-fold to about about 10-fold to about 50-fold, about 10-fold to about 45-fold, about 10-fold to about about 10-fold to about 35-fold, about 10-fold to about 30-fold, about 10-fold to about about 10-fold to about 20-fold, about 10-fold to about 15-fold, about 15-fold to about about 15-fold to about 75-fold, about 15-fold to about 70-fold, about 15-fold to about 65-fold, about 15-fold to about 60-fold, about 15-fold to about 55-fold, about 15-fold to about about 15-fold to about 45-fold, about 15-fold to about 40-fold, about 15-fold to about 35-fold, about 15-fold to about 30-fold, about 15-fold to about 25-fold, about 15-fold to about about 20-fold to about 80-fold, about 20-fold to about 75-fold, about 20-fold to about about 20-fold to about 65-fold, about 20-fold to about 60-fold, about 20-fold to about 55-fold, about 20-fold to about 50-fold, about 20-fold to about 45-fold, about 20-fold to about about 20-fold to about 35-fold, about 20-fold to about 30-fold, about 20-fold to about 25-fold, about 25-fold to about 80-fold, about 25-fold to about 75-fold, about 25-fold to about about 25-fold to about 65-fold, about 25-fold to about 60-fold, about 25-fold to about 55-fold, about 25-fold to about 50-fold, about 25-fold to about 45-fold, about 25-fold to about about 25-fold to about 35-fold, about 25-fold to about 30-fold, about 30-fold to about about 30-fold to about 75-fold, about 30-fold to about 70-fold, about 30-fold to about 65-fold, about 30-fold to about 60-fold, about 30-fold to about 55-fold, about 30-fold to about about 30-fold to about 45-fold, about 30-fold to about 40-fold, about 30-fold to about about 35-fold to about 80-fold, about 35-fold to about 75-fold, about 35-fold to about about 35-fold to about 65-fold, about 35-fold to about 60-fold, about 35-fold to about 55-fold, about 35-fold to about 50-fold, about 35-fold to about 45-fold, about 35-fold to about about 40-fold to about 80-fold, about 40-fold to about 75-fold, about 40-fold to about about 40-fold to about 65-fold, about 40-fold to about 60-fold, about 40-fold to about 55-fold, about 40-fold to about 50-fold, about 40-fold to about 45-fold, about 45-fold to about about 45-fold to about 75-fold, about 45-fold to about 70-fold, about 45-fold to about 65-fold, about 45-fold to about 60-fold, about 45-fold to about 55-fold, about 45-fold to about about 50-fold to about 80-fold, about 50-fold to about 75-fold, about 50-fold to about about 50-fold to about 65-fold, about 50-fold to about 60-fold, about 50-fold to about 85-fold, about 55-fold to about 80-fold, about 55-fold to about 75-fold, about 55-fold to about about 55-fold to about 65-fold, about 55-fold to about 60-fold, about 60-fold to about about 60-fold to about 75-fold, about 60-fold to about 70-fold, about 60-fold to about 75-fold, about 65-fold to about 80-fold, about 65-fold to about 75-fold, about 65-fold to about about 70-fold to about 80-fold, about 70-fold to about 75-fold, or about 75-fold to about expansion of NK cells at about day 10 after the contacting step.

In some embodiments, the NK cell is re-contacted (e.g., contacted for a second time) (e.g., for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, hours, 21 hours, 22 hours, 23 hours, 24 hours, 36 hours, 48 hours, 60 hours, 72 hours, 4 days, days, 6 days, 7 days, 8 days, 9 days, 10 days, 11 days or 12 days) with the first polypeptide, the second polypeptide and the third polypeptide, and optionally the fourth polypeptide, at about day 8 to about day 12 (e.g., about day 10) after an initial contacting step, and the methods described herein provide for an additional about 8-fold to about 80-fold (or any of the subranges of this range described herein) expansion of NK cells (e.g., for a total of about 64-fold to about 6400-fold (or any of the subranges in this range described herein) expansion of NK cells) at about day after the initial contacting step. In some embodiments, the methods described herein provide for at least about 64-fold expansion, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 1,100-fold, at least about 1,200-fold, at least about 1,300-fold, at least about 1,400-fold, at least about 1,500-fold, at least about 1,600-fold, at least about 1,700-fold, at least about 1,800-fold, at least about 1,900-fold, at least about 2,000-fold, at least about 2,100-fold, at least about 2,000-fold, at least about 2,100-fold, at least about 2,200-fold, at least about 2,300-fold, at least about 2,400-fold, at least about 2,500-fold, at least about 2,600-fold, at least about 2,700-fold, at least about 2,800-fold, at least about 2,900-fold, at least about 3,000-fold, at least about 3,100-fold, at least about 3,200-fold, at least about 3,300-fold, at least about 3,400-fold, at least about 3,500-fold, at least about 3,600-fold, at least about 3,700-fold, at least about 3,800-fold, at least about 3,900-fold, at least about 4,000-fold, at least about 4,100-fold, at least about 4,200-fold, at least about 4,300-fold, at least about 4,400-fold, at least about 4,500-fold, at least about 4,600-fold, at least about 4,700-fold, at least about 4,800-fold, at least about 4,900-fold, at least about 5,000-fold, at least about 5,100-fold, at least about 5,200-fold, at least about 5,300-fold, at least about at least about 5,500-fold, at least about 5,600-fold, at least about 5,700-fold, at least about 5,800-fold, at least about 5,900-fold, at least about 6,000-fold, at least about 6,100-fold, at least about 6,200-fold, at least about 6,300-fold, or at least about 6,400-fold NK cell expansion at about day 18 to about day 22 (e.g., at about day 20) after the initial contacting step. In some embodiments, the methods described herein provide for about 64-fold to about 6,400-fold (e.g., about 64-fold to about 6,200-fold, about 64-fold to about 6,000-fold, about 64-fold to about about 64-fold to about 5,600-fold, about 64-fold to about 5,400-fold, about 64-fold to about 5,200-fold, about 64-fold to about 5,000-fold, about 64-fold to about 4,800-fold, about 64-fold to about 4,600-fold, about 64-fold to about 4,400-fold, about 64-fold to about 4,200-fold, about 64-fold to about 4,000-fold, about 64-fold to about 3,800-fold, about 64-fold to about 3,600-fold, about 64-fold to about 3,400-fold, about 64-fold to about 3,200-fold, about 64-fold to about 3,000-fold, about 64-fold to about 2,800-fold, about 64-fold to about 2,600-fold, about 64-fold to about 2,400-fold, about 64-fold to about 2,200-fold, about 64-fold to about 2,000-fold, about 64-fold to about 1,800-fold, about 64-fold to about 1,600-fold, about 64-fold to about 1,400-fold, about 64-fold to about 1,200-fold, about 64-fold to about 1,000-fold, about 64-fold to about 800-fold, about 64-fold to about 600-fold, about 64-fold to about 400-fold, about 64-fold to about 200-fold, about 64-fold to about 100-fold, about 100-fold to about 6,400-fold, about 100-fold to about 6,200-fold, about 100-fold to about 6,000-fold, about 100-fold to about 5,800-fold, about 100-fold to about 5,600-fold, about 100-fold to about 5,400-fold, about 100-fold to about 5,200-fold, about 100-fold to about 5,000-fold, about 100-fold to about 4,800-fold, about 100-fold to about 4,600-fold, about 100-fold to about 4,400-fold, about 100-fold to about 4,200-fold, about 100-fold to about 4,000-fold, about 100-fold to about 3,800-fold, about 100-fold to about 3,600-fold, about 100-fold to about 3,400-fold, about 100-fold to about 3,200-fold, about 100-fold to about 3,000-fold, about 100-fold to about 2,800-fold, about 100-fold to about 2,600-fold, about 100-fold to about 2,400-fold, about 100-fold to about 2,200-fold, about 100-fold to about 2,000-fold, about 100-fold to about 1,800-fold, about 100-fold to about 1,600-fold, about 100-fold to about 1,400-fold, about 100-fold to about 1,200-fold, about 100-fold to about 1,000-fold, about 100-fold to about 800-fold, about 100-fold to about 600-fold, about 100-fold to about 400-fold, about 100-fold to about 200-fold, about 200-fold to about 6,400-fold, about 200-fold to about 6,200-fold, about 200-fold to about 6,000-fold, about 200-fold to about 5,800-fold, about 200-fold to about 5,600-fold, about 200-fold to about 5,400-fold, about 200-fold to about 5,200-fold, about 200-fold to about 5,000-fold, about 200-fold to about 4,800-fold, about 200-fold to about 4,600-fold, about 200-fold to about 4,400-fold, about 200-fold to about 4,200-fold, about 200-fold to about 4,000-fold, about 200-fold to about 3,800-fold, about 200-fold to about 3,600-fold, about 200-fold to about 3,400-fold, about 200-fold to about 3,200-fold, about 200-fold to about 3,000-fold, about 200-fold to about 2,800-fold, about 200-fold to about 2,600-fold, about 200-fold to about 2,400-fold, about 200-fold to about 2,200-fold, about 200-fold to about 2,000-fold, about 200-fold to about 1,800-fold, about 200-fold to about 1,600-fold, about 200-fold to about 1,400-fold, about 200-fold to about 1,200-fold, about 200-fold to about 1,000-fold, about 200-fold to about 800-fold, about 200-fold to about 600-fold, about 200-fold to about 400-fold, about 400-fold to about 6,400-fold, about 400-fold to about 6,200-fold, about 400-fold to about 6,000-fold, about 400-fold to about 5,800-fold, about 400-fold to about 5,600-fold, about 400-fold to about 5,400-fold, about 400-fold to about 5,200-fold, about 400-fold to about 5,000-fold, about 400-fold to about 4,800-fold, about 400-fold to about 4,600-fold, about 400-fold to about 4,400-fold, about 400-fold to about 4,200-fold, about 400-fold to about 4,000-fold, about 400-fold to about 3,800-fold, about 400-fold to about 3,600-fold, about 400-fold to about 3,400-fold, about 400-fold to about 3,200-fold, about 400-fold to about 3,000-fold, about 400-fold to about 2,800-fold, about 400-fold to about 2,600-fold, about 400-fold to about 2,400-fold, about 400-fold to about 2,200-fold, about 400-fold to about 2,000-fold, about 400-fold to about 1,800-fold, about 400-fold to about 1,600-fold, about 400-fold to about 1,400-fold, about 400-fold to about 1,200-fold, about 400-fold to about 1,000-fold, about 400-fold to about 800-fold, about 400-fold to about 600-fold, about 600-fold to about 6,400-fold, about 600-fold to about 6,200-fold, about 600-fold to about 6,000-fold, about 600-fold to about 5,800-fold, about 600-fold to about 5,600-fold, about 600-fold to about 5,400-fold, about 600-fold to about 5,200-fold, about 600-fold to about 5,000-fold, about 600-fold to about 4,800-fold, about 600-fold to about 4,600-fold, about 600-fold to about 4,400-fold, about 600-fold to about 4,200-fold, about 600-fold to about 4,000-fold, about 600-fold to about 3,800-fold, about 600-fold to about 3,600-fold, about 600-fold to about 3,400-fold, about 600-fold to about 3,200-fold, about 600-fold to about 3,000-fold, about 600-fold to about 2,800-fold, about 600-fold to about 2,600-fold, about 600-fold to about 2,400-fold, about 600-fold to about 2,200-fold, about 600-fold to about 2,000-fold, about 600-fold to about 1,800-fold, about 600-fold to about 1,600-fold, about 600-fold to about 1,400-fold, about 600-fold to about 1,200-fold, about 600-fold to about 1,000-fold, about 600-fold to about 800-fold, about 800-fold to about 6,400-fold, about 800-fold to about 6,200-fold, about 800-fold to about 6,000-fold, about 800-fold to about 5,800-fold, about 800-fold to about 5,600-fold, about 800-fold to about 5,400-fold, about 800-fold to about 5,200-fold, about 800-fold to about 5,000-fold, about 800-fold to about 4,800-fold, about 800-fold to about 4,600-fold, about 800-fold to about 4,400-fold, about 800-fold to about 4,200-fold, about 800-fold to about 4,000-fold, about 800-fold to about 3,800-fold, about 800-fold to about 3,600-fold, about 800-fold to about 3,400-fold, about 800-fold to about 3,200-fold, about 800-fold to about 3,000-fold, about 800-fold to about 2,800-fold, about 800-fold to about 2,600-fold, about 800-fold to about 2,400-fold, about 800-fold to about 2,200-fold, about 800-fold to about 2,000-fold, about 800-fold to about 1,800-fold, about 800-fold to about 1,600-fold, about 800-fold to about 1,400-fold, about 800-fold to about 1,200-fold, about 800-fold to about 1,000-fold, about 1,000-fold to about 6,400-fold, about 1,000-fold to about 6,200-fold, about 1,000-fold to about 6,000-fold, about 1,000-fold to about about 1,000-fold to about 5,600-fold, about 1,000-fold to about 5,400-fold, about 1,000-fold to about 5,200-fold, about 1,000-fold to about 5,000-fold, about 1,000-fold to about 4,800-fold, about 1,000-fold to about 4,600-fold, about 1,000-fold to about 4,400-fold, about 1,000-fold to about 4,200-fold, about 1,000-fold to about 4,000-fold, about 1,000-fold to about 3,800-fold, about 1,000-fold to about 3,600-fold, about 1,000-fold to about 3,400-fold, about 1,000-fold to about 3,200-fold, about 1,000-fold to about 3,000-fold, about 1,000-fold to about 2,800-fold, about 1,000-fold to about 2,600-fold, about 1,000-fold to about 2,400-fold, about 1,000-fold to about 2,200-fold, about 1,000-fold to about 2,000-fold, about 1,000-fold to about 1,800-fold, about 1,000-fold to about 1,600-fold, about 1,000-fold to about 1,400-fold, about 1,000-fold to about 1,200-fold, about 2,000-fold to about 6,400-fold, about 2,000-fold to about 6,200-fold, about 2,000-fold to about 6,000-fold, about 2,000-fold to about 5,800-fold, about 2,000-fold to about 5,600-fold, about 2,000-fold to about 5,400-fold, about 2,000-fold to about about 2,000-fold to about 5,000-fold, about 2,000-fold to about 4,800-fold, about 2,000-fold to about 4,600-fold, about 2,000-fold to about 4,400-fold, about 2,000-fold to about 4,200-fold, about 2,000-fold to about 4,000-fold, about 2,000-fold to about 3,800-fold, about 2,000-fold to about 3,600-fold, about 2,000-fold to about 3,400-fold, about 2,000-fold to about 3,200-fold, about 2,000-fold to about 3,000-fold, about 2,000-fold to about 2,800-fold, about 2,000-fold to about 2,600-fold, about 2,000-fold to about 2,400-fold, about 1,000-fold to about 2,200-fold, about 3,000-fold to about 6,400-fold, about 3,000-fold to about 6,200-fold, about 3,000-fold to about 6,000-fold, about 3,000-fold to about 5,800-fold, about 3,000-fold to about about 3,000-fold to about 5,400-fold, about 3,000-fold to about 5,200-fold, about 3,000-fold to about 5,000-fold, about 3,000-fold to about 4,800-fold, about 3,000-fold to about 4,600-fold, about 3,000-fold to about 4,400-fold, about 3,000-fold to about 4,200-fold, about 3,000-fold to about 4,000-fold, about 3,000-fold to about 3,800-fold, about 3,000-fold to about 3,600-fold, about 3,000-fold to about 3,400-fold, about 3,000-fold to about 3,200-fold, about 4,000-fold to about 6,400-fold, about 4,000-fold to about 6,200-fold, about 4,000-fold to about 6,000-fold, about 4,000-fold to about 5,800-fold, about 4,000-fold to about 5,600-fold, about 4,000-fold to about 5,400-fold, about 4,000-fold to about 5,200-fold, about 4,000-fold to about about 4,000-fold to about 4,800-fold, about 4,000-fold to about 4,600-fold, about 4,000-fold to about 4,400-fold, about 4,000-fold to about 4,200-fold, about 5,000-fold to about 6,400-fold, about 5,000-fold to about 6,200-fold, about 5,000-fold to about 6,000-fold, about to about 5,800-fold, about 5,000-fold to about 5,600-fold, about 5,000-fold to about about 5,000-fold to about 5,200-fold, about 6,000-fold to about 6,400-fold, or about 6,000-fold to about 6,200-fold) expansion of NK cells at about day 20 after the initial contacting step.

In some embodiments, the NK cell is re-contacted (e.g., contacted for a third time) (e.g., for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 4 days, 5 days, 6 days, 7 days, 8 days, 9 days, 10 days, 11 days or 12 days) with the first polypeptide, the second polypeptide and the third polypeptide, and optionally the fourth polypeptide, at about day 18 to about day 22 (e.g., about day 20), after an initial contacting step and the methods described herein provide for an additional about 8-fold to about 80-fold (or any of the subranges of this range described herein) expansion of NK cells (e.g., for a total of about 512-fold to about 512,000-fold (or any of the subranges in this range described herein) expansion of NK cells)) at about day 28 to about day 32 (e.g., at about day 30) after the initial contacting step. In some embodiments, the methods described herein provide for at least about a 512-fold, at least about a 1,000-fold, at least about 5,000-fold, at least about 10,000-fold, at least about 15,000-fold, at least about 20,000-fold, at least about 25,000-fold, at least about 30,000-fold, at least about at least about 40,000-fold, at least about 45,000-fold, at least about 50,000-fold, at least about 55,000-fold, at least about 60,000-fold, at least about 65,000-fold, at least about at least about 75,000-fold, at least about 80,000-fold, at least about 85,000-fold, at least about 90,000-fold, at least about 95,000-fold, at least about 100,000-fold, at least about 105,000-fold, at least about 110,000-fold, at least about 115,000-fold, at least about 120,000-fold, at least about 125,000-fold, at least about 130,000-fold, at least about 135,000-fold, at least about 140,000-fold, at least about 145,000-fold, at least about 150,000-fold, at least about 155,000-fold, at least about 160,000-fold, at least about 165,000-fold, at least about 170,000-fold, at least about 175,000-fold, at least about 180,000-fold, at least about 185,000-fold, at least about 190,000-fold, at least about 195,000-fold, at least about 200,000-fold, at least about 205,000-fold, at least about 210,000-fold, at least about 215,000-fold, at least about 220,000-fold, at least about 225,000-fold, at least about 230,000-fold, at least about 235,000-fold, at least about 240,000-fold, at least about 245,000-fold, at least about 250,000-fold, at least about 255,000-fold, at least about 260,000-fold, at least about 265,000-fold, at least about 270,000-fold, at least about 275,000-fold, at least about 280,000-fold, at least about 285,000-fold, at least about 290,000-fold, at least about 295,000-fold, at least about 300,000-fold, at least about 305,000-fold, at least about 310,000-fold, at least about 315,000-fold, at least about 320,000-fold, at least about 325,000-fold, at least about 330,000-fold, at least about 335,000-fold, at least about 340,000-fold, at least about 345,000-fold, at least about 350,000-fold, at least about 355,000-fold, at least about 360,000-fold, at least about 365,000-fold, at least about 370,000-fold, at least about 375,000-fold, at least about 380,000-fold, at least about 385,000-fold, at least about 390,000-fold, at least about 395,000-fold, at least about 400,000-fold, at least about 405,000-fold, at least about 410,000-fold, at least about 415,000-fold, at least about 420,000-fold, at least about 425,000-fold, at least about 430,000-fold, at least about 435,000-fold, at least about 440,000-fold, at least about 445,000-fold, at least about 450,000-fold, at least about 455,000-fold, at least about 460,000-fold, at least about 465,000-fold, at least about 470,000-fold, at least about 475,000-fold, at least about 480,000-fold, at least about 485,000-fold, at least about 490,000-fold, at least about 495,000-fold, at least about 500,000-fold, at least about 505,000-fold, at least about 510,000-fold, or at least about 512,000-fold NK cell expansion at about day 28 to about day 32 (e.g., at about day 30) after the initial contacting. In some embodiments, the methods described herein provide for about 512-fold to about 512,000-fold, about 512-fold to about 510,000-fold, about 512-fold to about 505,000-fold, about 512-fold to about 500,000-fold, about 512-fold to about 495,000-fold, about 512-fold to about 490,000-fold, about 512-fold to about 485,000-fold, about 512-fold to about 480,000-fold, about 512-fold to about 475,000-fold, about 512-fold to about 470,000-fold, about 512-fold to about 465,000-fold, about 512-fold to about 460,000-fold, about 512-fold to about 455,000-fold, about 512-fold to about 450,000-fold, about 512-fold to about 445,000-fold, about 512-fold to about 440,000-fold, about 512-fold to about 435,000-fold, about 512-fold to about 430,000-fold, about 512-fold to about 425,000-fold, about 512-fold to about 420,000-fold, about 512-fold to about 415,000-fold, about 512-fold to about 410,000-fold, about 512-fold to about 405,000-fold, about 512-fold to about 400,000-fold, about 512-fold to about 395,000-fold, about 512-fold to about 390,000-fold, about 512-fold to about 385,000-fold, about 512-fold to about 380,000-fold, about 512-fold to about 375,000-fold, about 512-fold to about 370,000-fold, about 512-fold to about 365,000-fold, about 512-fold to about 360,000-fold, about 512-fold to about 355,000-fold, about 512-fold to about 350,000-fold, about 512-fold to about 345,000-fold, about 512-fold to about 340,000-fold, about 512-fold to about 335,000-fold, about 512-fold to about 330,000-fold, about 512-fold to about 325,000-fold, about 512-fold to about 320,000-fold, about 512-fold to about 315,000-fold, about 512-fold to about 310,000-fold, about 512-fold to about 305,000-fold, about 512-fold to about 300,000-fold, about 512-fold to about 295,000-fold, about 512-fold to about 290,000-fold, about 512-fold to about 285,000-fold, about 512-fold to about 280,000-fold, about 512-fold to about 275,000-fold, about 512-fold to about 270,000-fold, about 512-fold to about 265,000-fold, about 512-fold to about 260,000-fold, about 512-fold to about 255,000-fold, about 512-fold to about 250,000-fold, about 512-fold to about 245,000-fold, about 512-fold to about 240,000-fold, about 512-fold to about 235,000-fold, about 512-fold to about 230,000-fold, about 512-fold to about 225,000-fold, about 512-fold to about 220,000-fold, about 512-fold to about 215,000-fold, about 512-fold to about 210,000-fold, about 512-fold to about 205,000-fold, about 512-fold to about 200,000-fold, about 512-fold to about 195,000-fold, about 512-fold to about 190,000-fold, about 512-fold to about 185,000-fold, about 512-fold to about 180,000-fold, about 512-fold to about 175,000-fold, about 512-fold to about 170,000-fold, about 512-fold to about 165,000-fold, about 512-fold to about 160,000-fold, about 512-fold to about 155,000-fold, about 512-fold to about 150,000-fold, about 512-fold to about 145,000-fold, about 512-fold to about 140,000-fold, about 512-fold to about 135,000-fold, about 512-fold to about 130,000-fold, about 512-fold to about 125,000-fold, about 512-fold to about 120,000-fold, about 512-fold to about 115,000-fold, about 512-fold to about 110,000-fold, about 512-fold to about 105,000-fold, about 512-fold to about 100,000-fold, about 512-fold to about 95,000-fold, about 512-fold to about 90,000-fold, about 512-fold to about 85,000-fold, about 512-fold to about 80,000-fold, about 512-fold to about 75,000-fold, about 512-fold to about 70,000-fold, about 512-fold to about 65,000-fold, about 512-fold to about 60,000-fold, about 512-fold to about 55,000-fold, about 512-fold to about 50,000-fold, about 512-fold to about 45,000-fold, about 512-fold to about 40,000-fold, about 512-fold to about 35,000-fold, about 512-fold to about 30,000-fold, about 512-fold to about 25,000-fold, about 512-fold to about 20,000-fold, about 512-fold to about 15,000-fold, about 512-fold to about 10,000-fold, about 512-fold to about 5,000-fold, about 512-fold to about 1,000-fold, about 1,000-fold to about 512,000-fold, about 1,000-fold to about 510,000-fold, about 1,000-fold to about 505,000-fold, about 1,000-fold to about 500,000-fold, about 1,000-fold to about 495,000-fold, about 1,000-fold to about 490,000-fold, about 1,000-fold to about 485,000-fold, about 1,000-fold to about 480,000-fold, about 1,000-fold to about 475,000-fold, about 1,000-fold to about 470,000-fold, about 1,000-fold to about 465,000-fold, about 1,000-fold to about 460,000-fold, about 1,000-fold to about 455,000-fold, about 1,000-fold to about 450,000-fold, about 1,000-fold to about 445,000-fold, about 1,000-fold to about 440,000-fold, about 1,000-fold to about 435,000-fold, about 1,000-fold to about 430,000-fold, about 1,000-fold to about 425,000-fold, about 1,000-fold to about 420,000-fold, about 1,000-fold to about 415,000-fold, about 1,000-fold to about 410,000-fold, about 1,000-fold to about 405,000-fold, about 1,000-fold to about 400,000-fold, about 1,000-fold to about 395,000-fold, about 1,000-fold to about 390,000-fold, about 1,000-fold to about 385,000-fold, about 1,000-fold to about 380,000-fold, about 1,000-fold to about 375,000-fold, about 1,000-fold to about 370,000-fold, about 1,000-fold to about 365,000-fold, about 1,000-fold to about 360,000-fold, about 1,000-fold to about 355,000-fold, about 1,000-fold to about 350,000-fold, about 1,000-fold to about 345,000-fold, about 1,000-fold to about 340,000-fold, about 1,000-fold to about 335,000-fold, about 1,000-fold to about 330,000-fold, about 1,000-fold to about 325,000-fold, about 1,000-fold to about 320,000-fold, about 1,000-fold to about 315,000-fold, about 1,000-fold to about 310,000-fold, about 1,000-fold to about 305,000-fold, about 1,000-fold to about 300,000-fold, about 1,000-fold to about 295,000-fold, about 1,000-fold to about 290,000-fold, about 1,000-fold to about 285,000-fold, about 1,000-fold to about 280,000-fold, about 1,000-fold to about 275,000-fold, about 1,000-fold to about 270,000-fold, about 1,000-fold to about 265,000-fold, about 1,000-fold to about 260,000-fold, about 1,000-fold to about 255,000-fold, about 1,000-fold to about 250,000-fold, about 1,000-fold to about 245,000-fold, about 1,000-fold to about 240,000-fold, about 1,000-fold to about 235,000-fold, about 1,000-fold to about 230,000-fold, about 1,000-fold to about 225,000-fold, about 1,000-fold to about 220,000-fold, about 1,000-fold to about 215,000-fold, about 1,000-fold to about 210,000-fold, about 1,000-fold to about 205,000-fold, about 1,000-fold to about 200,000-fold, about 1,000-fold to about 195,000-fold, about 1,000-fold to about 190,000-fold, about 1,000-fold to about 185,000-fold, about 1,000-fold to about 180,000-fold, about 1,000-fold to about 175,000-fold, about 1,000-fold to about 170,000-fold, about 1,000-fold to about 165,000-fold, about 1,000-fold to about 160,000-fold, about 1,000-fold to about 155,000-fold, about 1,000-fold to about 150,000-fold, about 1,000-fold to about 145,000-fold, about 1,000-fold to about 140,000-fold, about 1,000-fold to about 135,000-fold, about 1,000-fold to about 130,000-fold, about 1,000-fold to about 125,000-fold, about 1,000-fold to about 120,000-fold, about 1,000-fold to about 115,000-fold, about 1,000-fold to about 110,000-fold, about 1,000-fold to about 105,000-fold, about 1,000-fold to about 100,000-fold, about 1,000-fold to about 95,000-fold, about 1,000-fold to about 90,000-fold, about 1,000-fold to about 85,000-fold, about 1,000-fold to about 80,000-fold, about 1,000-fold to about 75,000-fold, about 1,000-fold to about 70,000-fold, about 1,000-fold to about 65,000-fold, about 1,000-fold to about 60,000-fold, about 1,000-fold to about 55,000-fold, about 1,000-fold to about 50,000-fold, about 1,000-fold to about 45,000-fold, about 1,000-fold to about 40,000-fold, about 1,000-fold to about 35,000-fold, about 1,000-fold to about 30,000-fold, about 1,000-fold to about 25,000-fold, about 1,000-fold to about 20,000-fold, about 1,000-fold to about 15,000-fold, about 1,000-fold to about 10,000-fold, about 1,000-fold to about 5,000-fold, about 10,000-fold to about 512,000-fold, about 10,000-fold to about 510,000-fold, about 10,000-fold to about 505,000-fold, about 10,000-fold to about 500,000-fold, about 10,000-fold to about 495,000-fold, about 10,000-fold to about 490,000-fold, about 10,000-fold to about 485,000-fold, about 10,000-fold to about 480,000-fold, about 10,000-fold to about 475,000-fold, about 10,000-fold to about 470,000-fold, about 10,000-fold to about 465,000-fold, about 10,000-fold to about 460,000-fold, about 10,000-fold to about 455,000-fold, about 10,000-fold to about 450,000-fold, about 10,000-fold to about 445,000-fold, about 10,000-fold to about 440,000-fold, about 10,000-fold to about 435,000-fold, about 10,000-fold to about 430,000-fold, about 10,000-fold to about 425,000-fold, about 10,000-fold to about 420,000-fold, about 10,000-fold to about 415,000-fold, about 10,000-fold to about 410,000-fold, about 10,000-fold to about 405,000-fold, about 10,000-fold to about 400,000-fold, about 10,000-fold to about 395,000-fold, about 10,000-fold to about 390,000-fold, about 10,000-fold to about 385,000-fold, about 10,000-fold to about 380,000-fold, about 10,000-fold to about 375,000-fold, about 10,000-fold to about 370,000-fold, about 10,000-fold to about 365,000-fold, about 10,000-fold to about 360,000-fold, about 10,000-fold to about 355,000-fold, about 10,000-fold to about 350,000-fold, about 10,000-fold to about 345,000-fold, about 10,000-fold to about 340,000-fold, about 10,000-fold to about 335,000-fold, about 10,000-fold to about 330,000-fold, about 10,000-fold to about 325,000-fold, about 10,000-fold to about 320,000-fold, about 10,000-fold to about 315,000-fold, about 10,000-fold to about 310,000-fold, about 10,000-fold to about 305,000-fold, about 10,000-fold to about 300,000-fold, about 10,000-fold to about 295,000-fold, about 10,000-fold to about 290,000-fold, about 10,000-fold to about 285,000-fold, about 10,000-fold to about 280,000-fold, about 10,000-fold to about 275,000-fold, about 10,000-fold to about 270,000-fold, about 10,000-fold to about 265,000-fold, about 10,000-fold to about 260,000-fold, about 10,000-fold to about 255,000-fold, about 10,000-fold to about 250,000-fold, about 10,000-fold to about 245,000-fold, about 10,000-fold to about 240,000-fold, about 10,000-fold to about 235,000-fold, about 10,000-fold to about 230,000-fold, about 10,000-fold to about 225,000-fold, about 10,000-fold to about 220,000-fold, about 10,000-fold to about 215,000-fold, about 10,000-fold to about 210,000-fold, about 10,000-fold to about 205,000-fold, about 10,000-fold to about 200,000-fold, about 10,000-fold to about 195,000-fold, about 10,000-fold to about 190,000-fold, about 10,000-fold to about 185,000-fold, about 10,000-fold to about 180,000-fold, about 10,000-fold to about 175,000-fold, about 10,000-fold to about 170,000-fold, about 10,000-fold to about 165,000-fold, about 10,000-fold to about 160,000-fold, about 10,000-fold to about 155,000-fold, about 10,000-fold to about 150,000-fold, about 10,000-fold to about 145,000-fold, about 10,000-fold to about 140,000-fold, about 10,000-fold to about 135,000-fold, about 10,000-fold to about 130,000-fold, about 10,000-fold to about 125,000-fold, about 10,000-fold to about 120,000-fold, about 10,000-fold to about 115,000-fold, about 10,000-fold to about 110,000-fold, about 10,000-fold to about 105,000-fold, about 10,000-fold to about 100,000-fold, about 10,000-fold to about 95,000-fold, about 10,000-fold to about 90,000-fold, about 10,000-fold to about about 10,000-fold to about 80,000-fold, about 10,000-fold to about 75,000-fold, about 10,000-fold to about 70,000-fold, about 10,000-fold to about 65,000-fold, about 10,000-fold to about 60,000-fold, about 10,000-fold to about 55,000-fold, about 10,000-fold to about about 10,000-fold to about 45,000-fold, about 10,000-fold to about 40,000-fold, about 10,000-fold to about 35,000-fold, about 10,000-fold to about 30,000-fold, about 10,000-fold to about 25,000-fold, about 10,000-fold to about 20,000-fold, about 10,000-fold to about about 100,000-fold to about 512,000-fold, about 100,000-fold to about 510,000-fold, about 100,000-fold to about 505,000-fold, about 100,000-fold to about 500,000-fold, about 100,000-fold to about 495,000-fold, about 100,000-fold to about 490,000-fold, about 100,000-fold to about 485,000-fold, about 100,000-fold to about 480,000-fold, about 100,000-fold to about 475,000-fold, about 100,000-fold to about 470,000-fold, about 100,000-fold to about 465,000-fold, about 100,000-fold to about 460,000-fold, about 100,000-fold to about 455,000-fold, about 100,000-fold to about 450,000-fold, about 100,000-fold to about 445,000-fold, about 100,000-fold to about 440,000-fold, about 100,000-fold to about 435,000-fold, about 100,000-fold to about 430,000-fold, about 100,000-fold to about 425,000-fold, about 100,000-fold to about 420,000-fold, about 100,000-fold to about 415,000-fold, about 100,000-fold to about 410,000-fold, about 100,000-fold to about 405,000-fold, about 100,000-fold to about 400,000-fold, about 100,000-fold to about 395,000-fold, about 100,000-fold to about 390,000-fold, about 100,000-fold to about 385,000-fold, about 100,000-fold to about 380,000-fold, about 100,000-fold to about 375,000-fold, about 100,000-fold to about 370,000-fold, about 100,000-fold to about 365,000-fold, about 100,000-fold to about 360,000-fold, about 100,000-fold to about 355,000-fold, about 100,000-fold to about 350,000-fold, about 100,000-fold to about 345,000-fold, about 100,000-fold to about 340,000-fold, about 100,000-fold to about 335,000-fold, about 100,000-fold to about 330,000-fold, about 100,000-fold to about 325,000-fold, about 100,000-fold to about 320,000-fold, about 100,000-fold to about 315,000-fold, about 100,000-fold to about 310,000-fold, about 100,000-fold to about 305,000-fold, about 100,000-fold to about 300,000-fold, about 100,000-fold to about 295,000-fold, about 100,000-fold to about 290,000-fold, about 100,000-fold to about 285,000-fold, about 100,000-fold to about 280,000-fold, about 100,000-fold to about 275,000-fold, about 100,000-fold to about 270,000-fold, about 100,000-fold to about 265,000-fold, about 100,000-fold to about 260,000-fold, about 100,000-fold to about 255,000-fold, about 100,000-fold to about 250,000-fold, about 100,000-fold to about 245,000-fold, about 100,000-fold to about 240,000-fold, about 100,000-fold to about 235,000-fold, about 100,000-fold to about 230,000-fold, about 100,000-fold to about 225,000-fold, about 100,000-fold to about 220,000-fold, about 100,000-fold to about 215,000-fold, about 100,000-fold to about 210,000-fold, about 100,000-fold to about 205,000-fold, about 100,000-fold to about 200,000-fold, about 100,000-fold to about 195,000-fold, about 100,000-fold to about 190,000-fold, about 100,000-fold to about 185,000-fold, about 100,000-fold to about 180,000-fold, about 100,000-fold to about 175,000-fold, about 100,000-fold to about 170,000-fold, about 100,000-fold to about 165,000-fold, about 100,000-fold to about 160,000-fold, about 100,000-fold to about 155,000-fold, about 100,000-fold to about 150,000-fold, about 100,000-fold to about 145,000-fold, about 100,000-fold to about 140,000-fold, about 100,000-fold to about 135,000-fold, about 100,000-fold to about 130,000-fold, about 100,000-fold to about 125,000-fold, about 100,000-fold to about 120,000-fold, about 100,000-fold to about 115,000-fold, about 100,000-fold to about 110,000-fold, about 100,000-fold to about 105,000-fold, about 200,000-fold to about 512,000-fold, about 200,000-fold to about 510,000-fold, about 200,000-fold to about 505,000-fold, about 200,000-fold to about 500,000-fold, about 200,000-fold to about 495,000-fold, about 200,000-fold to about 490,000-fold, about 200,000-fold to about 485,000-fold, about 200,000-fold to about 480,000-fold, about 200,000-fold to about 475,000-fold, about 200,000-fold to about 470,000-fold, about 200,000-fold to about 465,000-fold, about 200,000-fold to about 460,000-fold, about 200,000-fold to about 455,000-fold, about 200,000-fold to about 450,000-fold, about 200,000-fold to about 445,000-fold, about 200,000-fold to about 440,000-fold, about 200,000-fold to about 435,000-fold, about 200,000-fold to about 430,000-fold, about 200,000-fold to about 425,000-fold, about 200,000-fold to about 420,000-fold, about 200,000-fold to about 415,000-fold, about 200,000-fold to about 410,000-fold, about 200,000-fold to about 405,000-fold, about 200,000-fold to about 400,000-fold, about 200,000-fold to about 395,000-fold, about 200,000-fold to about 390,000-fold, about 200,000-fold to about 385,000-fold, about 200,000-fold to about 380,000-fold, about 200,000-fold to about 375,000-fold, about 200,000-fold to about 370,000-fold, about 200,000-fold to about 365,000-fold, about 200,000-fold to about 360,000-fold, about 200,000-fold to about 355,000-fold, about 200,000-fold to about 350,000-fold, about 200,000-fold to about 345,000-fold, about 200,000-fold to about 340,000-fold, about 200,000-fold to about 335,000-fold, about 200,000-fold to about 330,000-fold, about 200,000-fold to about 325,000-fold, about 200,000-fold to about 320,000-fold, about 200,000-fold to about 315,000-fold, about 200,000-fold to about 310,000-fold, about 200,000-fold to about 305,000-fold, about 200,000-fold to about 300,000-fold, about 200,000-fold to about 295,000-fold, about 200,000-fold to about 290,000-fold, about 200,000-fold to about 285,000-fold, about 200,000-fold to about 280,000-fold, about 200,000-fold to about 275,000-fold, about 200,000-fold to about 270,000-fold, about 200,000-fold to about 265,000-fold, about 200,000-fold to about 260,000-fold, about 200,000-fold to about 255,000-fold, about 200,000-fold to about 250,000-fold, about 200,000-fold to about 245,000-fold, about 200,000-fold to about 240,000-fold, about 200,000-fold to about 235,000-fold, about 200,000-fold to about 230,000-fold, about 200,000-fold to about 225,000-fold, about 200,000-fold to about 220,000-fold, about 200,000-fold to about 215,000-fold, about 200,000-fold to about 210,000-fold, about 200,000-fold to about 205,000-fold, about 300,000-fold to about 512,000-fold, about 300,000-fold to about 510,000-fold, about 300,000-fold to about 505,000-fold, about 300,000-fold to about 500,000-fold, about 300,000-fold to about 495,000-fold, about 300,000-fold to about 490,000-fold, about 300,000-fold to about 485,000-fold, about 300,000-fold to about 480,000-fold, about 300,000-fold to about 475,000-fold, about 300,000-fold to about 470,000-fold, about 300,000-fold to about 465,000-fold, about 300,000-fold to about 460,000-fold, about 300,000-fold to about 455,000-fold, about 300,000-fold to about 450,000-fold, about 300,000-fold to about 445,000-fold, about 300,000-fold to about 440,000-fold, about 300,000-fold to about 435,000-fold, about 300,000-fold to about 430,000-fold, about 300,000-fold to about 425,000-fold, about 300,000-fold to about 420,000-fold, about 300,000-fold to about 415,000-fold, about 300,000-fold to about 410,000-fold, about 300,000-fold to about 405,000-fold, about 300,000-fold to about 400,000-fold, about 300,000-fold to about 395,000-fold, about 300,000-fold to about 390,000-fold, about 300,000-fold to about 385,000-fold, about 300,000-fold to about 380,000-fold, about 300,000-fold to about 375,000-fold, about 300,000-fold to about 370,000-fold, about 300,000-fold to about 365,000-fold, about 300,000-fold to about 360,000-fold, about 300,000-fold to about 355,000-fold, about 300,000-fold to about 350,000-fold, about 300,000-fold to about 345,000-fold, about 300,000-fold to about 340,000-fold, about 300,000-fold to about 335,000-fold, about 300,000-fold to about 330,000-fold, about 300,000-fold to about 325,000-fold, about 300,000-fold to about 320,000-fold, about 300,000-fold to about 315,000-fold, about 300,000-fold to about 310,000-fold, about 300,000-fold to about 305,000-fold, about 400,000-fold to about 512,000-fold, about 400,000-fold to about 510,000-fold, about 400,000-fold to about 505,000-fold, about 400,000-fold to about 500,000-fold, about 400,000-fold to about 495,000-fold, about 400,000-fold to about 490,000-fold, about 400,000-fold to about 485,000-fold, about 400,000-fold to about 480,000-fold, about 400,000-fold to about 475,000-fold, about 400,000-fold to about 470,000-fold, about 400,000-fold to about 465,000-fold, about 400,000-fold to about 460,000-fold, about 400,000-fold to about 455,000-fold, about 400,000-fold to about 450,000-fold, about 400,000-fold to about 445,000-fold, about 400,000-fold to about 440,000-fold, about 400,000-fold to about 435,000-fold, about 400,000-fold to about 430,000-fold, about 400,000-fold to about 425,000-fold, about 400,000-fold to about 420,000-fold, about 400,000-fold to about 415,000-fold, about 400,000-fold to about 410,000-fold, about 400,000-fold to about 405,000-fold, about 500,000-fold to about 512,000-fold, about 500,000-fold to about 510,000-fold, or about 500,000-fold to about 505,000-fold expansion of NK cells at about day 30 after the initial contacting step.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### Methods of Expanding NK Cells

Provided herein are methods of expanding an NK cell including: contacting an NK cell disposed in a liquid culture medium, in the absence of a feeder cell, with (i) a first polypeptide comprising B7-H6 or a functional fragment thereof, or an anti-NKp30 antigen-binding domain, (ii) a second polypeptide comprising 4-1BBL or a functional fragment thereof, and (iii) a third polypeptide comprising ICAM-1 or a functional fragment thereof, and optionally (iv) a fourth polypeptide comprising IL-21 or a functional fragment thereof, under conditions sufficient for expansion of the NK cell.

B7-H6 belongs to the B7 family of co-stimulatory molecules involved in the immune response. B7-H6 is a ligand for the NK-cell-activating receptor NKp30 and is rarely expressed in healthy tissue, however, it is found to be overexpressed in several primary tumors including leukemia, lymphoma, and gastrointestinal stromal tumors (see, e.g., Brandt et al., J. Exp. Med. 206:1495-1503, 2009). In some embodiments, a first polypeptide comprises B7-H6 or a functional fragment thereof (e.g., a B7-H6 fragment capable of binding to NKp30, an extracellular domain of B7-H6 (e.g., SEQ ID NOs: 2, 4 and 26), or an IgV-like domain of B7-H6 (e.g., SEQ ID NO: 27 and 28). In some embodiments, a first polypeptide comprises an amino acid sequence at least 80%, at least 90%, at least 95%, at least 96%, at least 97%, at least 98%, at least 99% identical, or 100% identical to SEQ ID NO: 1 or 3. In some embodiments, a first polypeptide comprises an amino acid sequence of an extracellular domain of B7-H6 (e.g., an amino acid sequence that is at least 80%, at least 90%, at least 99%, or 100% identical to SEQ ID NO: 2 or 4. In some embodiments, a first polypeptide comprises an amino acid sequence of a truncated extracellular domain of human B7-H6 (e.g., an amino acid sequence that is at least 80%, at least 90%, at least 95%, at least 96%, at least 97%, at least 98%, at least 99% identical, or 100% identical to SEQ ID NO: 26). In some embodiments, a first polypeptide comprises an amino acid sequence of the Ig-V like domain of human B7-H6 (e.g., an amino acid sequence that is at least 80%, at least 90%, at least 95%, at least 96%, at least 97%, at least 98%, at least 99% identical, or 100% identical to SEQ ID NO: 27 or 28).

Mature Human B7-H6
(SEQ ID NO: 1)
DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVTPLKAQGTVQLEVVASPASRLLLDQVGMKENEDKYMCESSGFYPEAIN
ITWEKQTQKFPHPIEISEDVITGPTIKNMDGTFNVTSCLKLNSSQEDPGT
VYQCVVRHASLHTPLRSNFTLTAARHSLSETEKTDNFSIHWWPISFIGVG
LVLLIVLIPWKKICNKSSSAYTPLKCILKHWNSFDTQTLKKEHLIFFCTR
AWPSYQLQDGEAWPPEGSVNINTIQQLDVFCRQEGKWSEVPYVQAFFALR
DNPDLCQCCRIDPALLTVTSGKSIDDNSTKSEKQTPREHSDAVPDAPILP
VSPIWEPPPATTSTTPVLSSQPPTLLLPLQ
Extracellular Domain of Human B7-H6
(SEQ ID NO: 2)
DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVTPLKAQGTVQLEVVASPASRLLLDQVGMKENEDKYMCESSGFYPEAIN
ITWEKQTQKFPHPIEISEDVITGPTIKNMDGTFNVTSCLKLNSSQEDPGT
VYQCVVRHASLHTPLRSNFTLTAARHSLSETEKTDNFS
Truncated Extracellular Domain of Human B7-H6
(SEQ ID NO: 26)
DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVTPLKAQGTVQLEVVASPA
IgV-like Domain of Human B7-H6
(SEQ ID NO: 27)
KVEMMAGGTQITPLNDNVTIFCNIFYSQPLNITSMGITWFWKSLTFDKEV
KVFEFFGDHQEAFRPGAIVSPWRLKSGDASLRLPGIQLEEAGEYRCEVVV
TPLKAQGTVQLE
Mature Rat B7-H6
(SEQ ID NO: 3)
LPSAGGLELEMAGTTQIVFLHEDVTIPCKILGSLHLDLSIVGVIWSLKKD
GDESEVFKFYGDQLEAVRPGANVSLLGLEHGDASLYLPRFELWEAGEYQC
KVVVTPEKKEGTTRLEVVAHPNMSLSEKPATARGGKEKLIICQLDGFYPE
ALDIKWMGSALKDSHFQEITEGVVTGPTVKNDDGTFSVTSSLALKPALED
HMYQCVVWHRSWLMPQSLNVTVFENTRDSTHGTVPPTAEVGPPVSEPRSV
MIYVTIIGLCILLFSVIVCGLWKWKRLTTSNTGLCLRLDVRCCLPSFRNT
Extracellular Domain of Rat B7-H6
(SEQ ID NO: 4)
LPSAGGLELEMAGTTQIVFLHEDVTIPCKILGSLHLDLSIVGVIWSLKKD
GDESEVFKFYGDQLEAVRPGANVSLLGLEHGDASLYLPRFELWEAGEYQC
KVVVTPEKKEGTTRLEVVAHPNMSLSEKPATARGGKEKLIICQLDGFYPE
ALDIKWMGSALKDSHFQEITEGVVTGPTVKNDDGTFSVTSSLALKPALED
HMYQCVVWHRSWLMPQSLNVTVFENTRDSTHGTVPPTAEVGPPVSEPR
IgV-like Domain of Rat B7-H6
(SEQ ID NO: 28)
GGLELEMAGTTQIVFLHEDVTIPCKILGSLHLDLSIVGVIWSLKKDGDES
EVFKFYGDQLEAVRPGANVSLLGLEHGDASLYLPRFELWEAGEYQCKVVV
TPEKKEGTTRLE

In some embodiments, a first polypeptide comprises an anti-NKp30 antigen-binding domain. Non-limiting examples of a first polypeptide including an anti-NKp30 antigen-binding domain include an antibody or an antigen-binding fragment thereof. Non-limiting examples of anti-NKp30 antibodies are known in the art, including, e.g., anti-human NKp30/NCR3 monoclonal mouse antibody MAB18491 (clone #2108471, R&D Systems™); anti-NKp30 rabbit monoclonal antibody, clone number EPR23058-137 (ab243649; Abcam™), anti-NKp30 rabbit monoclonal antibody, clone number EPR14509 (ab250880; Abcam™), and anti-NKp30 rabbit polyclonal antibody (ab203390; Abcam™); anti-human CD337 (NKp30) mouse monoclonal antibody, cloneAF29-4D12 (Cat. No. 130-092-554; Miltenyi Biotec), anti-NKp30 mouse monoclonal antibody CLH9 (Cat. No. sc-33647; Santa Cruz Biotechnology, Inc.), BV510 mouse anti-human CD337 (NKp30) antibody (clone p30-15, Cat. No. 743170; BD Biosciences), and anti-NKp30 mouse monoclonal antibody CLH3 (Cat. No. sc-33646; Santa Cruz Biotechnology, Inc.). Non-limiting examples of first polypeptides that include an anti-NKp30 antigen-binding domain include an antibody (e.g., IgG1, IgG2, IgG3, IgG4), a VHH, an scFv, a Fab′ fragment, an Fv fragment, or a F(ab′)₂ fragment.

In some embodiments, a second polypeptide comprises 4-1BBL or a functional fragment thereof (e.g., a 4-1BBL fragment capable of binding to 4-1BB or an extracellular domain of 4-1BBL (e.g., SEQ ID NOs: 6, 8, and 10)). 4-1BBL, also known as tumor necrosis factor ligand superfamily member 9 (TNFSF9), is a type 2 transmembrane glycoprotein receptor found on antigen-presenting cells and binds to its cognate receptor 4-1BB. In some embodiments, a second polypeptide comprises an amino acid sequence at least 80%, at least 90%, at least 95%, at least 96%, at least 97%, at least 98%, at least 99% identical, or 100% identical to SEQ ID NO: 5, 7, or 9. In some embodiments, a second polypeptide comprises an amino acid sequence of an extracellular domain of 4-1BBL (e.g., an amino acid sequence that is at least 80%, at least 90%, at least 95%, at least 96%, at least 97%, at least 98%, at least 99%, or 100% identical to SEQ ID NO: 6, 8, or 10).

Mature Human 4-1BBL
(SEQ ID NO: 5)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE
Extracellular Domain of Human 4-1BBL
(SEQ ID NO: 6)
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE
Mature Mouse 4-1BBL
(SEQ ID NO: 7)
MDQHTLDVEDTADARHPAGTSCPSDAALLRDTGLLADAALLSDTVRPTNA
ALPTDAAYPAVNVRDREAAWPPALNFCSRHPKLYGLVALVLLLLIAACVP
IFTRTEPRPALTITTSPNLGTRENNADQVTPVSHIGCPNTTQQGSPVFAK
LLAKNQASLCNTTLNWHSQDGAGSSYLSQGLRYEEDKKELVVDSPGLYYV
FLELKLSPTFTNTGHKVQGWVSLVLQAKPQVDDFDNLALTVELFPCSMEN
LKVDRSWSQLLLLKAGHRLSVGLRAYLHGAQDAYRDWELSYPNTTSFGLF
LVKPDNPWE
Extracellular Domain of Mouse 4-1BBL
(SEQ ID NO: 8)
RTEPRPALTITTSPNLGTRENNADQVTPVSHIGCPNTTQQGSPVFAKLLA
KNQASLCNTTLNWHSQDGAGSSYLSQGLRYEEDKKELVVDSPGLYYVFLE
LKLSPTFTNTGHKVQGWVSLVLQAKPQVDDFDNLALTVELFPCSMENKLV
DRSWSQLLLLKAGHRLSVGLRAYLHGAQDAYRDWELSYPNTTSFGLFLVK
PDNPWE
Mature Rat 4-1BBL
(SEQ ID NO: 9)
MDQHALDVGGTVDAGHPPGTAQPPDAAFLRDTGLPSDAAFLVDTVRLTEA
ALPTDAACPAVNVRDPEAAWPPALNFCSRHPRLYCLGGLVLLLLVACVPI
FIRILPQPALTITTSPDLDTGENTADQVTPVSHIGCANTTQQGFPVFAKL
LAKNQASLSNTTLNWHSQEGAGSSYLSRGLRYEEDKKELVVDSPGLYYVF
LELKLSPTSINTGRKVQGWVSLVLQAKPQVDHLDSLALTVELFPCSMEDK
LVDRSWNRLLRLKAGHRLSVDLRAYLHGAQDAYRDWELSHANTTSFGLFL
VKPDPPQG
Extracellular Domain of Rat 4-1BBL
(SEQ ID NO: 10)
RILPQPALTITTSPDLDTGENTADQVTPVSHIGCANTTQQGFPVFAKLLA
KNQASLSNTTLNWHSQEGAGSSYLSRGLRYEEDKKELVVDSPGLYYVFLE
LKLSPTSINTGRKVQGWVSLVLQAKPQVDHLDSLALTVELFPCSMEDKLV
DRSWNRLLRLKAGHRLSVDLRAYLHGAQDAYRDWELSHANTTSFGLFLVK
PDPPQG

In some embodiments, a third polypeptide comprises ICAM-1 or a functional fragment thereof (e.g., an ICAM-1 fragment capable of binding to leukocyte function-associated antigen-1 (LFA-1), an extracellular domain of ICAM-1 (e.g., SEQ ID NOs: 12, 14 and 16), or an ICAM-1 Domain 1 (e.g., SEQ ID NO: 29)). Intracellular adhesion molecule 1 (ICAM-1) is part of the immunoglobulin superfamily and is a cell surface glycoprotein. ICAM-1 is known to have roles in cell-cell adhesion, extravasation, and signal transduction, including recruitment of immune cells such as macrophages and granulocytes (see, e.g., Etienne-Manneville et al., J. Immunol. 163(2):668-74, 1999). In some embodiments, a third polypeptide comprises an amino acid sequence at least 80%, at least 90%, at least 95%, at least 96%, at least 97%, at least 98%, at least 99% identical, or 100% identical to SEQ ID NO: 11, 13, or 15. In some embodiments, a third polypeptide comprises an amino acid sequence of an extracellular domain of ICAM-1 (e.g., an amino acid sequence that is at least 80%, at least 90%, at least 95%, at least 96%, at least 97%, at least 98%, at least 99%, or 100% identical to SEQ ID NO: 12, 14, or 16. In some embodiments, a third polypeptide comprises an amino acid sequence of a Domain 1 of ICAM-1 (e.g., an amino acid sequence that is at least 80%, at least 90%, at least 95%, at least 96%, at least 97%, at least 98%, at least 99% identical, or 100% identical to SEQ ID NO: 29).

Mature Human ICAM-1
(SEQ ID NO: 11)
QTSVSPSKVILPRGGSVLVTCSTSCDQPKLLGIETPLPKKELLLPGNNRK
VYELSNVQEDSQPMCYSNCPDGQSTAKTFLTVYWTPERVELAPLPSWQPV
GKNLTLRCQVEGGAPRANLTVVLLRGEKELKREPAVGEPAEVTTTVLVRR
DHHGANFSCRTELDLRPQGLELFENTSAPYQLQTFVLPATPPQLVSPRVL
EVDTQGTVVCSLDGLFPVSEAQVHLALGDQRLNPTVTYGNDSFSAKASVS
VTAEDEGTQRLTCAVILGNQSQETLQTVTIYSFPAPNVILTKPEVSEGTE
VTVKCEAHPRAKVTLNGVPAQPLGPRAQLLLKATPEDNGRSFSCSATLEV
AGQLIHNQTRELRVLYGPRLDERDCPGNWTWPENSQQTPMCQAKWGNPLP
ELKCLKDGTFPLPIGESVTVTRDLEGTYLCRARSTQGEVTRKVTVNVLSP
RYEIVIITVVAAAVIMGTAGLSTYLYNRQRKIKKYRLQQAQKGTPMKPNT
QATPP
Extracellular Domain of Human ICAM-1
(SEQ ID NO: 12)
QTSVSPSKVILPRGGSVLVTCSTSCDQPKLLGIETPLPKKELLLPGNNRK
VYELSNVQEDSQPMCYSNCPDGQSTAKTFLTVYWTPERVELAPLPSWQPV
GKNLTLRCQVEGGAPRANLTVVLLRGEKELKREPAVGEPAEVTTTVLVRR
DHHGANFSCRTELDLRPQGLELFENTSAPYQLQTFVLPATPPQLVSPRVL
EVDTQGTVVCSLDGLFPVSEAQVHLALGDQRLNPTVTYGNDSFSAKASVS
VTAEDEGTQRLTCAVILGNQSQETLQTVTIYSFPAPNVILTKPEVSEGTE
VTVKCEAHPRAKVTLNGVPAQPLGPRAQLLLKATPEDNGRSFSCSATLEV
AGQLIHKNQTRELRVLYGPRLDERDCPGNWTWPENSQQTPMCQAWGNPLP
ELK
Mature Mouse ICAM-1
(SEQ ID NO: 13)
QVSIHPREAFLPQGGSVQVNCSSSCKEDLSLGLETQWLKDELESGPNWKL
FELSEIGEDSSPLCFENCGTVQSSASATITVYSFPESVELRPLPAWQQVG
KDLTLRCHVDGGAPRTQLSAVLLRGEEILSRQPVGGHPKDPKEITFTVLA
SRGDHGANFSCRTELDLRPQGLALFSNVSEARSLRTFDLPATIPKLDTPD
LLEVGTQQKLFCSLEGLFPASEARIYLELGGQMPTQESTNSSDSVSATAL
VEVTEEFDRTLPLRCVLELADQILETQRTLTVYNFSAPVLTLSQLEVSEG
SQVTVKCEAHSGSKVVLLSGVEPRPPTPQVQFTLNASSEDHKRSFFCSAA
LEVAGKFLFKNQTLELHVLYGPRLDETDCLGNWTWQEGSQQTLKCQAWGN
PSPKMTCRRKADGALLPIGVVKSVKQEMNGTYVCHAFSSHGNVTRNVYLT
VLYHSQNNWTIIILVPVLLVIVGLVMAASYVYNRQRKIRIYKLQKAQEEA
IKLKGQAPPP
Extracellular Domain of Mouse ICAM-1
(SEQ ID NO: 14)
QVSIHPREAFLPQGGSVQVNCSSSCKEDLSLGLETQWLKDELESGPNWKL
FELSEIGEDSSPLCFENCGTVQSSASATITVYSFPESVELRPLPAWQQVG
KDLTLRCHVDGGAPRTQLSAVLLRGEEILSRQPVGGHPKDPKEITFTVLA
SRGDHGANFSCRTELDLRPQGLALFSNVSEARSLRTFDLPATIPKLDTPD
LLEVGTQQKLFCSLEGLFPASEARIYLELGGQMPTQESTNSSDSVSATAL
VEVTEEFDRTLPLRCVLELADQILETQRTLTVYNFSAPVLTLSQLEVSEG
SQVTVKCEAHSGSKVVLLSGVEPRPPTPQVQFTLNASSEDHKRSFFCSAA
LEVAGKFLFKNQTLELHVLYGPRLDETDCLGNWTWQEGSQQTLKCQAWGN
PSPKMTCRRKADGALLPIGVVKSVKQEMNGTYVCHAFSSHGNVTRNVYLT
VLYHSQNN
Mature Rat ICAM-1
(SEQ ID NO: 15)
QVSIHPTEAFLPRGGSVQVNCSSSCEDENLGLGLETNWMKDELSSGHNWK
LFKLSDIGEDSRPLCFENCGTTQSSASATITVYSFPERVELDPLPAWQQV
GKNLILRCLVEGGAPRTQLSVVLLRGNETLSRQAVDGDPKEITFTVLASR
GDHGANFSCFTELDLRPQGLSLFKNVSEVRQLRTFDLPTRVLKLDTPDLL
EVGTQQKFLCSLEGLFPASEAQIYLEMGGQMLTLESTNSRDFVSATASVE
VTEKLDRTLQLRCVLELADQTLEMEKTLRIYNFSAPILTLSQPEVSEGDQ
VTVKCEAHGGAQVVLLNSTSPRPPTSQGTSPRPPTSQIQFTLNASPEDHK
RRFFCSAALEVDGKSLFKNQTLELHVLYGPHLDKKDCLGNWTWQEGSQQT
LTCQPQGNPAPNLTCSRKADGVPLPIGMVKSVKREMNGTYKCRAFSSRGS
ITRDVHLTVLYHDQNTWVIIVGVLVLIIAGFVIVASIYTYYRQRKIRIYK
LQKAQEEALKLKVQAPPP
Extracellular Domain of Rat ICAM-1
(SEQ ID NO: 16)
QVSIHPTEAFLPRGGSVQVNCSSSCEDENLGLGLETNWMKDELSSGHNWK
LFKLSDIGEDSRPLCFENCGTTQSSASATITVYSFPERVELDPLPAWQQV
GKNLILRCLVEGGAPRTQLSVVLLRGNETLSRQAVDGDPKEITFTVLASR
GDHGANFSCFTELDLRPQGLSLFKNVSEVRQLRTFDLPTRVLKLDTPDLL
EVGTQQKFLCSLEGLFPASEAQIYLEMGGQMLTLESTNSRDFVSATASVE
VTEKLDRTLQLRCVLELADQTLEMEKTLRIYNFSAPILTLSQPEVSEGDQ
VTVKCEAHGGAQVVLLNSTSPRPPTSQGTSPRPPTSQIQFTLNASPEDHK
RRFFCSAALEVDGKSLFKNQTLELHVLYGPHLDKKDCLGNWTWQEGSQQT
LTCQPQGNPAPNLTCSRKADGVPLPIGMVKSVKREMNGTYKCRAFSSRGS
ITRDVHLTVLYHDQN
ICAM-1 Domain 1 (41-103 aa)
(SEQ ID NO: 29)
GGSVQVNCSSSCEDENLGLGLETNWMKDELSSGHNWKLFKLSDIGEDSRP
LCFENCGTTQSSA

In some embodiments, a fourth polypeptide comprises IL-21 or a functional fragment thereof (e.g., an IL-21 fragment capable of binding to its cognate receptor complex composed of the IL-21 receptor private chain (IL-21Ra) and the common γ-chain (γC)). In some embodiments, a fourth polypeptide comprises an amino acid sequence at least 80%, at least 90%, at least 95%, at least 96%, at least 97%, at least 98%, at least 99% identical, or 100% identical to SEQ ID NO: 23, 24, or 25.

Mature Human IL-21
(SEQ ID NO: 23)
HKSSSQGQDRHMIRMRQLIDIVDQLKNYVNDLVPEFLPAPEDVETNCEWS
AFSCFQKAQLKSANTGNNERIINVSIKKLKRKPPSTNAGRRQKHRLTCPS
CDSYEKKPPKEFLERFKSLLQKMIHQHLSSRTHGSEDS
Mature Mouse IL-21
(SEQ ID NO: 24)
HKSSPQGPDRLLIRLRHLIDIVEQLKIYENDLDPELLSAPQDVKGHCEHA
AFACFQKAKLKPSNPGNNKTFIIDLVAQLRRRLPARRGGKKQKHIAKCPS
CDSYEKRTPKEFLERLKWLLQKMIHQHLS
Mature Rat IL-21
(SEQ ID NO: 25)
HKSSPQRPDHLLIRLRHLMDIVEQLKIYENDLDPELLTAPQDVKGQCEHE
AFACFQKAKLKPSNTGNNKTFINDLLAQLRRRLPAKRTGNKQRHMAKCPS
CDLYEKKTPKEFLERLKWLLQKMIHQHLS

In some embodiments, the NK cell is periodically re-contacted with the first polypeptide, the second polypeptide, and the third polypeptide, and optionally, the fourth polypeptide. In some embodiments, the methods described herein are performed for about 1, about 5, about 10, about 15, about 20, about 25, about 30, about 35, about 40, about 45, about 50, about 55, or about 60 days. In some embodiments, the methods provided herein are performed for about 1 day to about 60 days, about 1 day to about 55 days, about 1 day to about 50 days, about 1 day to about 45 days, about 1 day to about 40 days, about 1 day to about 35 days, about 1 day to about 30 days, about 1 day to about 25 days, about 1 day to about 20 days, about 1 day to about 15 days, about 1 day to about 10 days, about 1 day to about 8 days, about 1 day to about 6 days, about 1 day to about 4 days, about 1 day to about 2 days, about 2 days to about 60 days, about 2 days to about 55 days, about 2 days to about 50 days, about 2 days to about 45 days, about 2 days to about 40 days, about 2 days to about 35 days, about 2 days to about 30 days, about 2 days to about 25 days, about 2 days to about 20 days, about 2 days to about 15 days, about 2 days to about 10 days, about 2 days to about 8 days, about 2 days to about 6 days, about 2 days to about 4 days, about 4 days to about 60 days, about 4 days to about 55 days, about 4 days to about 50 days, about 4 days to about 45 days, about 4 days to about 40 days, about 4 days to about 35 days, about 4 days to about 30 days, about 4 days to about 25 days, about 4 days to about 20 days, about 4 days to about 15 days, about 4 days to about 10 days, about 4 days to about 8 days, about 4 days to about 6 days, about 6 days to about 60 days, about 6 days to about 55 days, about 6 days to about 50 days, about 6 days to about 45 days, about 6 days to about 40 days, about 6 days to about 35 days, about 6 days to about 30 days, about 6 days to about 25 days, about 6 days to about 20 days, about 6 days to about 15 days, about 6 days to about 10 days, about 6 days to about 8 days, about 8 days to about 60 days, about 8 days to about 55 days, about 8 days to about days, about 8 days to about 45 days, about 8 days to about 40 days, about 8 days to about 35 days, about 8 days to about 30 days, about 8 days to about 25 days, about 8 days to about 20 days, about 8 days to about 15 days, about 8 days to about 10 days, about 10 days to about 60 days, about 10 days to about 55 days, about 10 days to about 50 days, about 10 days to about 45 days, about 10 days to about 40 days, about 10 days to about 35 days, about 10 days to about 30 days, about 10 days to about 25 days, about 10 days to about 20 days, about 10 days to about 15 days, about 15 days to about 60 days, about 15 days to about 55 days, about 15 days to about 50 days, about 15 days to about 45 days, about 15 days to about 40 days, about 15 days to about 35 days, about 15 days to about 30 days, about 15 days to about 25 days, about 15 days to about 20 days, about 20 days to about 60 days, about 20 days to about 55 days, about 20 days to about 50 days, about 20 days to about 45 days, about 20 days to about 40 days, about 20 days to about 35 days, about 20 days to about 30 days, about 20 days to about 25 days, about 25 days to about 60 days, about 25 days to about 55 days, about 25 days to about 50 days, about 25 days to about 45 days, about 25 days to about 40 days, about 25 days to about 35 days, about 25 days to about 30 days, about 30 days to about 60 days, about 30 days to about 55 days, about 30 days to about 50 days, about 30 days to about 45 days, about 30 days to about 40 days, about 30 days to about 35 days, about 35 days to about 60 days, about 35 days to about 55 days, about 35 days to about 50 days, about 35 days to about 45 days, about 35 days to about 40 days, about 40 days to about 60 days, about 40 days to about 55 days, about 40 days to about 50 days, about 40 days to about 45 days, about 45 days to about 60 days, about 45 days to about 55 days, about 45 days to about 50 days, about 50 days to about 60 days, about 50 days to about 55 days, or about 55 days to about days.

In some embodiments, the NK cell is re-contacted with the first polypeptide, the second polypeptide, and the third polypeptide, and optionally the fourth polypeptide, at about day 1, day 2, day 3, day 4, day 5, day 10, day 15, day 20, day 25, day 30, day 35, day 40, day 45, day 50, day 55, and/or day 60 after an initial contacting (e.g., an initial contacting step). In some embodiments, the NK cell is re-contacted with the first polypeptide, the second polypeptide, and the third polypeptide, and optionally the fourth polypeptide, at about day 1, day 2, day 3, day 4, day 5, day 6, day 9, day 12, day 15, day 18, day 21, day 24, day 27, day 30, day 33, day 36, day 39, day 42, day 45, day 48, day 51, day 54, day 57, and/or day 60 after an initial contacting (e.g., an initial contacting step). In some embodiments, the NK cell is re-contacted with the first polypeptide, the second polypeptide, and the third polypeptide, and optionally the fourth polypeptide, at about day 1, day 2, day 3, day 4, day 5, day 8, day 12, day 16, day 20, day 24, day 28, day 32, day 36, day 40, day 44, day 48, day 52, day 56, and/or day 60 after an initial contacting (e.g., an initial contacting step). In some embodiments, the NK cell is re-contacted with the first polypeptide, the second polypeptide, and the third polypeptide, and optionally the fourth polypeptide, at about day 1, day 2, day 3, day 4, day 5, day 6, day 12, day 18, day 24, day day 36, day 42, day 48, day 54, and/or day 60 after an initial contacting (e.g., an initial contacting step). In some embodiments, the NK cell is re-contacted with the first polypeptide, the second polypeptide, and the third polypeptide, and optionally the fourth polypeptide, at about day 1, day 2, day 3, day 4, day 5, day 7, day 14, day 21, day 28, day 35, day 42, day 49, and/or day 56 after an initial contacting (e.g., an initial contacting step). In some embodiments, the NK cell is re-contacted with the first polypeptide, the second polypeptide, and the third polypeptide, and optionally the fourth polypeptide, at about day 1, day 2, day 3, day 4, day 5, day 8, day 16, day 24, day 32, day 40, day 48, and/or day 56 after an initial contacting (e.g., an initial contacting step). In some embodiments, the NK cell is re-contacted with the first polypeptide, the second polypeptide, and the third polypeptide, and optionally the fourth polypeptide, at about day 1, day 2, day 3, day 4, day 5, day 9, day 18, day 27, day 36, day 45, and/or day 54 after an initial contacting (e.g., an initial contacting step). In some embodiments, the NK cell is re-contacted with the first polypeptide, the second polypeptide, and the third polypeptide, and optionally the fourth polypeptide, at about day 1, day 2, day 3, day 4, day 5, day 10, day 20, day day 40, day 50, and/or day 60 after an initial contacting (e.g., an initial contacting step).

In some embodiments of any of the methods provided herein, the NK cell is contacted with the NK cell with the first polypeptide, the second polypeptide, the third polypeptide, and optionally the fourth polypeptide, for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, hours, 72 hours, 4 days, 5 days, 6 days, 7 days, 8 days, 9 days, 10 days, 11 days or 12 days.

In some embodiments of any of the methods provided herein, the NK cell is re-contacted with the NK cell with the first polypeptide, the second polypeptide, the third polypeptide, and optionally the fourth polypeptide, for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, hours, 72 hours, 4 days, 5 days, 6 days, 7 days, 8 days, 9 days, 10 days, 11 days or 12 days.

In some embodiments, the methods described herein are performed for about 30 days, and the NK cell is re-contacted with the first polypeptide, the second polypeptide, and the third polypeptide, and optionally the fourth polypeptide, at about day 10 and at about day 20 after an initial contacting (e.g., an initial contacting step).

In some embodiments, one or more of the first polypeptide, the second polypeptide, and the third polypeptide are present in a liquid culture medium. In some embodiments, two or more of the first polypeptide, the second polypeptide, and the third polypeptide are present in a liquid culture medium. In some embodiments, the first polypeptide, the second polypeptide, and the third polypeptide are present in a liquid culture medium.

In some embodiments, the liquid culture medium includes a chemically-defined liquid culture medium. For example, suitable liquid culture mediums include AIM-V medium (Thermo Fisher Scientific), NK MACS medium (Miltenyi Biotec), EL837 medium (Biomedical EliteCell Corp.), X-VIVO 10 medium (Lonza), X-VIVO 15 medium (Lonza), X-VIVO 20 medium (Lonza), LGM-3 lymphocyte growth medium (Lonza), and LYMPHOONE T-cell expansion xeno-free medium (Takara). In some embodiments, the liquid culture medium is AIM-V. In some embodiments, the liquid culture medium includes additional reagents. For example, additional reagents can include serum, a serum replacement (e.g., CTS Immune Cell SR serum replacement (Thermo Fisher Scientific), KNOCKOUT serum replacement (Thermo Fisher Scientific), Panexin NTS Pharma Grade (PAN Biotech GmbH)), penicillin, streptomycin, HEPES (4-(2-hydroxyethyl)-1-piperazineethanesulfonic acid), and/or L-alanyl-L-glutamine.

In some embodiments, the liquid culture medium includes interleukin IL-2 or a functional fragment thereof. IL-2 is a type of cytokine signaling molecule in the immune system that regulates the activities of white blood cells responsible for immunity. In some embodiments, the IL-2 or a functional fragment thereof comprises an amino acid sequence that is at least 80%, at least 90%, at least 95%, at least 96%, at least 97%, at least 98%, at least 99%, or 100% identical to SEQ ID NO: 17, 18, or 19.

Mature Human IL-2
(SEQ ID NO: 17)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT
Mature Mouse IL-2
(SEQ ID NO: 18)
APTSSSTSSSTAEAQQQQQQQQQQQQHLEQLLMDLQELLSRMENYRNLKL
PRMLTFKFYLPKQATELKDLQCLEDELGPLRHVLDLTQSKSFQLEDAENF
ISNIRVTVVKLKGSDNTFECQFDDESATVVDFLRRWIAFCQSIISTSPQ
Mature Rat IL-2
(SEQ ID NO: 19)
APTSSPAKETQQHLEQLLLDLQVLLRGIDNYKNLKLPMMLTFKFYLPKQA
TELKHLQCLENELGALQRVLDLTQSKSFHLEDAGNFISNIRVTVVKLKGS
ENKFECQFDDEPATVVEFLRRWIAICQSIISTMTQ

In some embodiments, the liquid culture medium includes IL-18 or a functional fragment thereof. In some embodiments, the liquid culture medium includes both IL-2 or a functional fragment thereof and IL-18 or a functional fragment thereof. IL-18 is a proinflammatory cytokine and belongs to the IL-1 superfamily and is produced mainly by macrophages. In some embodiments, the IL-18 or a functional fragment thereof comprises an amino acid sequence that is at least 80%, at least 90%, at least 95%, at least 96%, at least 97%, at least 98%, at least 99%, or 100% identical to SEQ ID NO: 20, 21, or 22.

Human IL-18 (active form)
(SEQ ID NO: 20)
YFGKLESKLSVIRNLNDQVLFIDQGNRPLFEDMTDSDCRDNAPRTIFIIS
MYKDSQPRGMAVTISVKCEKISTLSCENKIISFKEMNPPDNIKDTKSDII
FFQRSVPGHDNKMQFESSSYEGYFLACEKERDLFKLILKKEDELGDRSIM
FTVQNED
Mouse IL-18 (active form)
(SEQ ID NO: 21)
NFGRLHCTTAVIRNINDQVLFVDKRQPVFEDMTDIDQSASEPQTRLIIYM
YKDSEVRGLAVTLSVKDSKMSTLSCKNKIISFEEMDPPENIDDIQSDLIF
FQKRVPGHNKMEFESSLYEGHFLACQKEDDAFKLILKKKDENGDKSVMFT
LTNLHQS
Rat IL-18 (active form)
(SEQ ID NO: 22)
HFGRLHCTTAVIRSINDQVLFVDKRNPPVFEDMPDIDRTANESQTRLIIY
MYKDSEVRGLAVTLSVKDGRMSTLSCKNKIISFEEMNPPENIDDIKSDLI
FFQKRVPGHNKMEFESSLYEGHFLACQKEDDAFKLVLKRKDENGDKSVMF
TLTNLHQS

In some embodiments, the liquid culture medium includes from about 10 U/mL, about 15 U/mL, about 20 U/mL, about 25 U/mL, about 30 U/mL, about 35 U/mL, about 40 U/mL, about U/mL, about 50 U/mL, about 55 U/mL, about 60 U/mL, about 65 U/mL, about 70 U/mL, about 75 U/mL, about 80 U/mL, about 85 U/mL, about 90 U/mL, about 95 U/mL, about 100 U/mL, about 105 U/mL, about 110 U/mL, about 115 U/mL, about 120 U/mL, about 125 U/mL, about 130 U/mL, about 135 U/mL, about 140 U/mL, about 145 U/mL, about 150 U/mL, about 155 U/mL, about 160 U/mL, about 165 U/mL, about 170 U/mL, about 175 U/mL, about 180 U/mL, about 185 U/mL, about 190 U/mL, about 195 U/mL, about 200 U/mL, about 205 U/mL, about 210 U/mL, about 215 U/mL, about 220 U/mL, about 225 U/mL, about 230 U/mL, about 235 U/mL, about 240 U/mL, about 245 U/mL, about 250 U/mL, about 255 U/mL, about 260 U/mL, about 265 U/mL, about 270 U/mL, about 275 U/mL, about 280 U/mL, about 285 U/mL, about 290 U/mL, about 295 U/mL, about 300 U/mL, about 305 U/mL, about 310 U/mL, about 315 U/mL, about 320 U/mL, about 325 U/mL, about 330 U/mL, about 335 U/mL, about 340 U/mL, about 345 U/mL, about 350 U/mL, about 355 U/mL, about 360 U/mL, about 365 U/mL, about 370 U/mL, about 375 U/mL, about 380 U/mL, about 385 U/mL, about 390 U/mL, about 395 U/mL, about 400 U/mL, about 405 U/mL, about 410 U/mL, about 415 U/mL, about 420 U/mL, about 425 U/mL, about 430 U/mL, about 435 U/mL, about 440 U/mL, about 445 U/mL, about 450 U/mL, about 455 U/mL, about 460 U/mL, about 465 U/mL, about 470 U/mL, about 475 U/mL, about 480 U/mL, about 485 U/mL, about 490 U/mL, about 495 U/mL, or about 500 U/mL, about 505 U/mL, about 510 U/mL, about 515 U/mL, about 520 U/mL, about 525 U/mL, about 530 U/mL, about 535 U/mL, about 540 U/mL, about 545 U/mL, about 550 U/mL, about 555 U/mL, about 560 U/mL, about 565 U/mL, about 570 U/mL, about 575 U/mL, about 580 U/mL, about 585 U/mL, about 590 U/mL, about 595 U/mL, about 600 U/mL, about 605 U/mL, about 610 U/mL, about 615 U/mL, about 620 U/mL, about 625 U/mL, about 630 U/mL, about 635 U/mL, about 640 U/mL, about 645 U/mL, about 650 U/mL, about 655 U/mL, about 660 U/mL, about 665 U/mL, about 670 U/mL, about 675 U/mL, about 680 U/mL, about 685 U/mL, about 690 U/mL, about 695 U/mL, about 700 U/mL, about 705 U/mL, about 710 U/mL, about 715 U/mL, about 720 U/mL, about 725 U/mL, about 730 U/mL, about 735 U/mL, about 740 U/mL, about 745 U/mL, about 750 U/mL, about 755 U/mL, about 760 U/mL, about 765 U/mL, about 770 U/mL, about 775 U/mL, about 780 U/mL, about 785 U/mL, about 790 U/mL, about 795 U/mL, about 800 U/mL, about 805 U/mL, about 810 U/mL, about 815 U/mL, about 820 U/mL, about 825 U/mL, about 830 U/mL, about 835 U/mL, about 840 U/mL, about 845 U/mL, about 850 U/mL, about 855 U/mL, about 860 U/mL, about 865 U/mL, about 870 U/mL, about 875 U/mL, about 880 U/mL, about 885 U/mL, about 890 U/mL, about 895 U/mL, about 900 U/mL, about 905 U/mL, about 910 U/mL, about 915 U/mL, about 920 U/mL, about 925 U/mL, about 930 U/mL, about 935 U/mL, about 940 U/mL, about 945 U/mL, about 950 U/mL, about 955 U/mL, about 960 U/mL, about 965 U/mL, about 970 U/mL, about 975 U/mL, about 980 U/mL, about 985 U/mL, about 990 U/mL, about 995 U/mL, about 1000 U/mL, about 1005 U/mL, about 1010 U/mL, about 1015 U/mL, about 1020 U/mL, about 1025 U/mL, about 1030 U/mL, about 1035 U/mL, about 1040 U/mL, about 1045 U/mL, about 1050 U/mL, about 1055 U/mL, about 1060 U/mL, about 1065 U/mL, about 1070 U/mL, about 1075 U/mL, about 1080 U/mL, about 1085 U/mL, about 1090 U/mL, about 1095 U/mL, about 1100 U/mL, about 1105 U/mL, about 1110 U/mL, about 1115 U/mL, about 1120 U/mL, about 1125 U/mL, about 1130 U/mL, about 1135 U/mL, about 1140 U/mL, about 1145 U/mL, about 1150 U/mL, about 1155 U/mL, about 1160 U/mL, about 1165 U/mL, about 1170 U/mL, about 1175 U/mL, about 1180 U/mL, about 1185 U/mL, about 1190 U/mL, about 1195 U/mL, about 1200 U/mL, about 1205 U/mL, about 1210 U/mL, about 1215 U/mL, about 1220 U/mL, about 1225 U/mL, about 1230 U/mL, about 1235 U/mL, about 1240 U/mL, about 1245 U/mL, about 1250 U/mL, about 1255 U/mL, about 1260 U/mL, about 1265 U/mL, about 1270 U/mL, about 1275 U/mL, about 1280 U/mL, about 1285 U/mL, about 1290 U/mL, about 1295 U/mL, about 1300 U/mL, about 1305 U/mL, about 1310 U/mL, about 1315 U/mL, about 1320 U/mL, about 1325 U/mL, about 1330 U/mL, about 1335 U/mL, about 1340 U/mL, about 1345 U/mL, about 1350 U/mL, about 1355 U/mL, about 1360 U/mL, about 1365 U/mL, about 1370 U/mL, about 1375 U/mL, about 1380 U/mL, about 1385 U/mL, about 1390 U/mL, about 1395 U/mL, about 1400 U/mL, about 1405 U/mL, about 1410 U/mL, about 1415 U/mL, about 1420 U/mL, about 1425 U/mL, about 1430 U/mL, about 1435 U/mL, about 1440 U/mL, about 1445 U/mL, about 1450 U/mL, about 1455 U/mL, about 1460 U/mL, about 1465 U/mL, about 1470 U/mL, about 1475 U/mL, about 1480 U/mL, about 1485 U/mL, about 1490 U/mL, about 1495 U/mL, or about 1500 U/mL of IL-2 or a functional fragment thereof.

In some embodiments, the liquid culture medium includes from about 10 U/mL to about 1500 U/mL, about 10 U/mL to about 1490 U/mL, about 10 U/mL to about 1480 U/mL, about 10 U/mL to about 1470 U/mL, about 10 U/mL to about 1460 U/mL, about 10 U/mL to about 1450 U/mL, about 10 U/mL to about 1440 U/mL, about 10 U/mL to about 1430 U/mL, about 10 U/mL to about 1420 U/mL, about 10 U/mL to about 1410 U/mL, about 10 U/mL to about 1400 U/mL, about 10 U/mL to about 1390 U/mL, about 10 U/mL to about 1380 U/mL, about 10 U/mL to about 1370 U/mL, about 10 U/mL to about 1360 U/mL, about 10 U/mL to about 1350 U/mL, about 10 U/mL to about 1340 U/mL, about 10 U/mL to about 1330 U/mL, about 10 U/mL to about 1320 U/mL, about 10 U/mL to about 1310 U/mL, about 10 U/mL to about 1300 U/mL, about 10 U/mL to about 1290 U/mL, about 10 U/mL to about 1280 U/mL, about 10 U/mL to about 1270 U/mL, about 10 U/mL to about 1260 U/mL, about 10 U/mL to about 1250 U/mL, about 10 U/mL to about 1240 U/mL, about 10 U/mL to about 1230 U/mL, about 10 U/mL to about 1220 U/mL, about 10 U/mL to about 1210 U/mL, about 10 U/mL to about 1200 U/mL, about 10 U/mL to about 1190 U/mL, about 10 U/mL to about 1180 U/mL, about 10 U/mL to about 1170 U/mL, about 10 U/mL to about 1160 U/mL, about 10 U/mL to about 1150 U/mL, about 10 U/mL to about 1140 U/mL, about 10 U/mL to about 1130 U/mL, about 10 U/mL to about 1120 U/mL, about 10 U/mL to about 1110 U/mL, about 10 U/mL to about 1100 U/mL, about 10 U/mL to about 1090 U/mL, about 10 U/mL to about 1080 U/mL, about 10 U/mL to about 1070 U/mL, about 10 U/mL to about 1060 U/mL, about 10 U/mL to about 1050 U/mL, about 10 U/mL to about 1040 U/mL, about 10 U/mL to about 1030 U/mL, about 10 U/mL to about 1020 U/mL, about 10 U/mL to about 1010 U/mL, about 10 U/mL to about 1000 U/mL, about 10 U/mL to about 990 U/mL, about 10 U/mL to about 980 U/mL, about 10 U/mL to about 970 U/mL, about 10 U/mL to about 960 U/mL, about 10 U/mL to about 950 U/mL, about 10 U/mL to about 940 U/mL, about 10 U/mL to about 930 U/mL, about 10 U/mL to about 920 U/mL, about 10 U/mL to about 910 U/mL, about 10 U/mL to about 900 U/mL, about 10 U/mL to about 890 U/mL, about 10 U/mL to about 880 U/mL, about 10 U/mL to about 870 U/mL, about 10 U/mL to about 860 U/mL, about 10 U/mL to about 850 U/mL, about 10 U/mL to about 840 U/mL, about 10 U/mL to about 830 U/mL, about 10 U/mL to about 820 U/mL, about 10 U/mL to about 810 U/mL, about 10 U/mL to about 800 U/mL, about 10 U/mL to about 790 U/mL, about 10 U/mL to about 780 U/mL, about 10 U/mL to about 770 U/mL, about 10 U/mL to about 760 U/mL, about 10 U/mL to about 750 U/mL, about 50 U/mL to about 1500 U/mL, about 50 U/mL to about 1490 U/mL, about 50 U/mL to about 1480 U/mL, about 50 U/mL to about 1470 U/mL, about 50 U/mL to about 1460 U/mL, about 50 U/mL to about 1450 U/mL, about 50 U/mL to about 1440 U/mL, about 50 U/mL to about 1430 U/mL, about 50 U/mL to about 1420 U/mL, about 50 U/mL to about 1410 U/mL, about 50 U/mL to about 1400 U/mL, about 50 U/mL to about 1390 U/mL, about 50 U/mL to about 1380 U/mL, about 50 U/mL to about 1370 U/mL, about 50 U/mL to about 1360 U/mL, about 50 U/mL to about 1350 U/mL, about 50 U/mL to about 1340 U/mL, about 50 U/mL to about 1330 U/mL, about 50 U/mL to about 1320 U/mL, about 50 U/mL to about 1310 U/mL, about 50 U/mL to about 1300 U/mL, about 50 U/mL to about 1290 U/mL, about 50 U/mL to about 1280 U/mL, about 50 U/mL to about 1270 U/mL, about 50 U/mL to about 1260 U/mL, about 50 U/mL to about 1250 U/mL, about 50 U/mL to about 1240 U/mL, about 50 U/mL to about 1230 U/mL, about 50 U/mL to about 1220 U/mL, about 50 U/mL to about 1210 U/mL, about 50 U/mL to about 1200 U/mL, about 50 U/mL to about 1190 U/mL, about 50 U/mL to about 1180 U/mL, about 50 U/mL to about 1170 U/mL, about 50 U/mL to about 1160 U/mL, about 50 U/mL to about 1150 U/mL, about 50 U/mL to about 1140 U/mL, about 50 U/mL to about 1130 U/mL, about 50 U/mL to about 1120 U/mL, about 50 U/mL to about 1110 U/mL, about 50 U/mL to about 1100 U/mL, about 50 U/mL to about 1090 U/mL, about 50 U/mL to about 1080 U/mL, about 50 U/mL to about 1070 U/mL, about 50 U/mL to about 1060 U/mL, about 50 U/mL to about 1050 U/mL, about 50 U/mL to about 1040 U/mL, about 50 U/mL to about 1030 U/mL, about 50 U/mL to about 1020 U/mL, about 50 U/mL to about 1010 U/mL, about 50 U/mL to about 1000 U/mL, about 50 U/mL to about 990 U/mL, about 50 U/mL to about 980 U/mL, about 50 U/mL to about 970 U/mL, about 50 U/mL to about 960 U/mL, about 50 U/mL to about 950 U/mL, about 50 U/mL to about 940 U/mL, about 50 U/mL to about 930 U/mL, about 50 U/mL to about 920 U/mL, about 50 U/mL to about 910 U/mL, about 50 U/mL to about 900 U/mL, about 50 U/mL to about 890 U/mL, about 50 U/mL to about 880 U/mL, about 50 U/mL to about 870 U/mL, about 50 U/mL to about 860 U/mL, about 50 U/mL to about 850 U/mL, about 50 U/mL to about 840 U/mL, about 50 U/mL to about 830 U/mL, about 50 U/mL to about 820 U/mL, about 50 U/mL to about 810 U/mL, about 50 U/mL to about 800 U/mL, about 50 U/mL to about 790 U/mL, about 50 U/mL to about 780 U/mL, about 50 U/mL to about 770 U/mL, about 50 U/mL to about 760 U/mL, about 50 U/mL to about 750 U/mL, about 100 U/mL to about 1500 U/mL, about 100 U/mL to about 1490 U/mL, about 100 U/mL to about 1480 U/mL, about 100 U/mL to about 1470 U/mL, about 100 U/mL to about 1460 U/mL, about 100 U/mL to about 1450 U/mL, about 100 U/mL to about 1440 U/mL, about 100 U/mL to about 1430 U/mL, about 100 U/mL to about 1420 U/mL, about 100 U/mL to about 1410 U/mL, about 100 U/mL to about 1400 U/mL, about 100 U/mL to about 1390 U/mL, about 100 U/mL to about 1380 U/mL, about 100 U/mL to about 1370 U/mL, about 100 U/mL to about 1360 U/mL, about 100 U/mL to about 1350 U/mL, about 100 U/mL to about 1340 U/mL, about 100 U/mL to about 1330 U/mL, about 100 U/mL to about 1320 U/mL, about 100 U/mL to about 1310 U/mL, about 100 U/mL to about 1300 U/mL, about 100 U/mL to about 1290 U/mL, about 100 U/mL to about 1280 U/mL, about 100 U/mL to about 1270 U/mL, about 100 U/mL to about 1260 U/mL, about 100 U/mL to about 1250 U/mL, about 100 U/mL to about 1240 U/mL, about 100 U/mL to about 1230 U/mL, about 100 U/mL to about 1220 U/mL, about 100 U/mL to about 1210 U/mL, about 100 U/mL to about 1200 U/mL, about 100 U/mL to about 1190 U/mL, about 100 U/mL to about 1180 U/mL, about 100 U/mL to about 1170 U/mL, about 100 U/mL to about 1160 U/mL, about 100 U/mL to about 1150 U/mL, about 100 U/mL to about 1140 U/mL, about 100 U/mL to about 1130 U/mL, about 100 U/mL to about 1120 U/mL, about 100 U/mL to about 1110 U/mL, about 100 U/mL to about 1100 U/mL, about 100 U/mL to about 1090 U/mL, about 100 U/mL to about 1080 U/mL, about 100 U/mL to about 1070 U/mL, about 100 U/mL to about 1060 U/mL, about 100 U/mL to about 1050 U/mL, about 100 U/mL to about 1040 U/mL, about 100 U/mL to about 1030 U/mL, about 100 U/mL to about 1020 U/mL, about 100 U/mL to about 1010 U/mL, about 100 U/mL to about 1000 U/mL, about 100 U/mL to about 990 U/mL, about 100 U/mL to about 980 U/mL, about 100 U/mL to about 970 U/mL, about 100 U/mL to about 960 U/mL, about 100 U/mL to about 950 U/mL, about 100 U/mL to about 940 U/mL, about 100 U/mL to about 930 U/mL, about 100 U/mL to about 920 U/mL, about 100 U/mL to about 910 U/mL, about 100 U/mL to about 900 U/mL, about 100 U/mL to about 890 U/mL, about 100 U/mL to about 880 U/mL, about 100 U/mL to about 870 U/mL, about 100 U/mL to about 860 U/mL, about 100 U/mL to about 850 U/mL, about 100 U/mL to about 840 U/mL, about 100 U/mL to about 830 U/mL, about 100 U/mL to about 820 U/mL, about 100 U/mL to about 810 U/mL, about 100 U/mL to about 800 U/mL, about 100 U/mL to about 790 U/mL, about 100 U/mL to about 780 U/mL, about 100 U/mL to about 770 U/mL, about 100 U/mL to about 760 U/mL, about 100 U/mL to about 750 U/mL, about 200 U/mL to about 1500 U/mL, about 200 U/mL to about 1490 U/mL, about 200 U/mL to about 1480 U/mL, about 200 U/mL to about 1470 U/mL, about 200 U/mL to about 1460 U/mL, about 200 U/mL to about 1450 U/mL, about 200 U/mL to about 1440 U/mL, about 200 U/mL to about 1430 U/mL, about 200 U/mL to about 1420 U/mL, about 200 U/mL to about 1410 U/mL, about 200 U/mL to about 1400 U/mL, about 200 U/mL to about 1390 U/mL, about 200 U/mL to about 1380 U/mL, about 200 U/mL to about 1370 U/mL, about 200 U/mL to about 1360 U/mL, about 200 U/mL to about 1350 U/mL, about 200 U/mL to about 1340 U/mL, about 200 U/mL to about 1330 U/mL, about 200 U/mL to about 1320 U/mL, about 200 U/mL to about 1310 U/mL, about 200 U/mL to about 1300 U/mL, about 200 U/mL to about 1290 U/mL, about 200 U/mL to about 1280 U/mL, about 200 U/mL to about 1270 U/mL, about 200 U/mL to about 1260 U/mL, about 200 U/mL to about 1250 U/mL, about 200 U/mL to about 1240 U/mL, about 200 U/mL to about 1230 U/mL, about 200 U/mL to about 1220 U/mL, about 200 U/mL to about 1210 U/mL, about 200 U/mL to about 1200 U/mL, about 200 U/mL to about 1190 U/mL, about 200 U/mL to about 1180 U/mL, about 200 U/mL to about 1170 U/mL, about 200 U/mL to about 1160 U/mL, about 200 U/mL to about 1150 U/mL, about 200 U/mL to about 1140 U/mL, about 200 U/mL to about 1130 U/mL, about 200 U/mL to about 1120 U/mL, about 200 U/mL to about 1110 U/mL, about 200 U/mL to about 1100 U/mL, about 200 U/mL to about 1090 U/mL, about 200 U/mL to about 1080 U/mL, about 200 U/mL to about 1070 U/mL, about 200 U/mL to about 1060 U/mL, about 200 U/mL to about 1050 U/mL, about 200 U/mL to about 1040 U/mL, about 200 U/mL to about 1030 U/mL, about 200 U/mL to about 1020 U/mL, about 200 U/mL to about 1010 U/mL, about 200 U/mL to about 1000 U/mL, about 200 U/mL to about 990 U/mL, about 200 U/mL to about 980 U/mL, about 200 U/mL to about 970 U/mL, about 200 U/mL to about 960 U/mL, about 200 U/mL to about 950 U/mL, about 200 U/mL to about 940 U/mL, about 200 U/mL to about 930 U/mL, about 200 U/mL to about 920 U/mL, about 200 U/mL to about 910 U/mL, about 200 U/mL to about 900 U/mL, about 200 U/mL to about 890 U/mL, about 200 U/mL to about 880 U/mL, about 200 U/mL to about 870 U/mL, about 200 U/mL to about 860 U/mL, about 200 U/mL to about 850 U/mL, about 200 U/mL to about 840 U/mL, about 200 U/mL to about 830 U/mL, about 200 U/mL to about 820 U/mL, about 200 U/mL to about 810 U/mL, about 200 U/mL to about 800 U/mL, about 200 U/mL to about 790 U/mL, about 200 U/mL to about 780 U/mL, about 200 U/mL to about 770 U/mL, about 200 U/mL to about 760 U/mL, or about 200 U/mL to about 750 U/mL of IL-2 or a functional fragment thereof.

In some embodiments, the liquid culture medium includes about 0.05 nM, about 0.1 nM, about 0.15 nM, about 0.2 nM, about 0.25 nM, about 0.3 nM, about 0.35 nM, about 0.4 nM, about nM, about 0.5 nM, about 0.55 nM, about 0.6 nM, about 0.65 nM, about 0.7 nM, about 0.75 nM, about 0.8 nM, about 0.85 nM, about 0.9 nM, about 0.95 nM, about 1 nM, about 1.05 nM, about 1.1 nM, about 1.15 nM, about 1.2 nM, about 1.25 nM, about 1.3 nM, about 1.35 nM, about 1.4 nM, about 1.45 nM, about 1.5 nM, about 1.55 nM, about 1.6 nM, about 1.65 nM, about 1.7 nM, about 1.75 nM, about 1.8 nM, about 1.85 nM, about 1.9 nM, about 1.95 nM, about 2 nM, about 2.05 nM, about 2.1 nM, about 2.15 nM, about 2.2 nM, about 2.25 nM, about 2.3 nM, about 2.35 nM, about 2.4 nM, about 2.45 nM, about 2.5 nM, about 2.55 nM, about 2.6 nM, about 2.65 nM, about 2.7 nM, about 2.75 nM, about 2.8 nM, about 2.85 nM, about 2.9 nM, about 2.95 nM, about 3 nM, about 3.05 nM, about 3.1 nM, about 3.15 nM, about 3.2 nM, about 3.25 nM, about 3.3 nM, about 3.35 nM, about 3.4 nM, about 3.45 nM, about 3.5 nM, about 3.55 nM, about 3.6 nM, about 3.65 nM, about 3.7 nM, about 3.75 nM, about 3.8 nM, about 3.85 nM, about 3.9 nM, about 3.95 nM, about 4 nM, from about 0.05 nM to about 4 nM, about 0.05 nM to about 3.5 nM, about 0.05 nM to about 3 nM, about 0.05 nM to about 2.5 nM, about 0.05 nM to about 2 nM, about 0.05 nM to about 1.5 nM, about 0.05 nM to about 1 nM, about 0.05 nM to about 0.9 nM, about 0.05 nM to about 0.8 nM, about 0.05 nM to about 0.7 nM, about 0.05 nM to about 0.6 nM, about 0.05 nM to about 0.5 nM, about 0.05 nM to about 0.4 nM, about 0.05 nM to about 0.3 nM, about 0.05 nM to about 0.2 nM, about 0.05 nM to about 0.1 nM, about 0.1 nM to about 4 nM, about 0.1 nM to about 3.5 nM, about 0.1 nM to about 3 nM, about 0.1 nM to about 2.5 nM, about nM to about 2 nM, about 0.1 nM to about 1.5 nM, about 0.1 nM to about 1 nM, about 0.1 nM to about 0.9 nM, about 0.1 nM to about 0.8 nM, about 0.1 nM to about 0.7 nM, about 0.1 nM to about 0.6 nM, about 0.1 nM to about 0.5 nM, about 0.1 nM to about 0.4 nM, about 0.1 nM to about 0.3 nM, about 0.1 nM to about 0.2 nM, about 0.2 nM to about 4 nM, about 0.2 nM to about 3.5 nM, about 0.2 nM to about 3 nM, about 0.2 nM to about 2.5 nM, about 0.2 nM to about 2 nM, about 0.2 nM to about 1.5 nM, about 0.2 nM to about 1 nM, about 0.2 nM to about 0.9 nM, about 0.2 nM to about 0.8 nM, about 0.2 nM to about 0.7 nM, about 0.2 nM to about 0.6 nM, about 0.2 nM to about 0.5 nM, about 0.2 nM to about 0.4 nM, about 0.2 nM to about 0.3 nM, about 0.2 nM to about 0.2 nM, or about 0.2 nM to about 0.1 nM of IL-18 or a functional fragment thereof.

In some embodiments, the liquid culture medium includes about 0.05 nM, about 0.1 nM, about 0.15 nM, about 0.2 nM, about 0.25 nM, about 0.3 nM, about 0.35 nM, about 0.4 nM, about nM, about 0.5 nM, about 0.55 nM, about 0.6 nM, about 0.65 nM, about 0.7 nM, about 0.75 nM, about 0.8 nM, about 0.85 nM, about 0.9 nM, about 0.95 nM, about 1 nM, about 1.05 nM, about 1.1 nM, about 1.15 nM, about 1.2 nM, about 1.25 nM, about 1.3 nM, about 1.35 nM, about 1.4 nM, about 1.45 nM, about 1.5 nM, about 1.55 nM, about 1.6 nM, about 1.65 nM, about 1.7 nM, about 1.75 nM, about 1.8 nM, about 1.85 nM, about 1.9 nM, about 1.95 nM, about 2 nM, about 2.05 nM, about 2.1 nM, about 2.15 nM, about 2.2 nM, about 2.25 nM, about 2.3 nM, about 2.35 nM, about 2.4 nM, about 2.45 nM, about 2.5 nM, about 2.55 nM, about 2.6 nM, about 2.65 nM, about 2.7 nM, about 2.75 nM, about 2.8 nM, about 2.85 nM, about 2.9 nM, about 2.95 nM, about 3 nM, about 3.05 nM, about 3.1 nM, about 3.15 nM, about 3.2 nM, about 3.25 nM, about 3.3 nM, about 3.35 nM, about 3.4 nM, about 3.45 nM, about 3.5 nM, about 3.55 nM, about 3.6 nM, about 3.65 nM, about 3.7 nM, about 3.75 nM, about 3.8 nM, about 3.85 nM, about 3.9 nM, about 3.95 nM, about 4 nM, about 0.1 nM to about 4 nM, about 0.1 nM to about 3.9 nM, about nM to about 3.8 nM, about 0.1 nM to about 3.7 nM, about 0.1 nM to about 3.6 nM, about 0.1 nM to about 3.5 nM, about 0.1 nM to about 3.4 nM, about 0.1 nM to about 3.3 nM, about 0.1 nM to about 3.2 nM, about 0.1 nM to about 3.1 nM, about 0.1 nM to about 3 nM, about 0.1 nM to about 2.9 nM, about 0.1 nM to about 2.8 nM, about 0.1 nM to about 2.7 nM, about 0.1 nM to about 2.6 nM, about 0.1 nM to about 2.5 nM, about 0.1 nM to about 2.4 nM, about 0.1 nM to about 2.3 nM, about 0.1 nM to about 2.2 nM, about 0.1 nM to about 2.1 nM, about 0.1 nM to about 2 nM, about 0.1 nM to about 1.9 nM, about 0.1 nM to about 1.8 nM, about 0.1 nM to about 1.7 nM, about 0.1 nM to about 1.6 nM, about 0.1 nM to about 1.5 nM, about 0.5 nM to about 4 nM, about 0.5 nM to about 3.9 nM, about 0.5 nM to about 3.8 nM, about 0.5 nM to about 3.7 nM, about 0.5 nM to about 3.6 nM, about 0.5 nM to about 3.5 nM, about 0.5 nM to about 3.4 nM, about 0.5 nM to about 3.3 nM, about 0.5 nM to about 3.2 nM, about 0.5 nM to about 3.1 nM, about 0.5 nM to about 3 nM, about 0.5 nM to about 2.9 nM, about 0.5 nM to about 2.8 nM, about 0.5 nM to about 2.7 nM, about 0.5 nM to about 2.6 nM, about 0.5 nM to about 2.5 nM, about 0.5 nM to about 2.4 nM, about 0.5 nM to about 2.3 nM, about 0.5 nM to about 2.2 nM, about 0.5 nM to about 2.1 nM, about 0.5 nM to about 2 nM, about 0.5 nM to about 1.9 nM, about nM to about 1.8 nM, about 0.5 nM to about 1.7 nM, about 0.5 nM to about 1.6 nM, about 0.5 nM to about 1.5 nM, about 1 nM to about 4 nM, about 1 nM to about 3.9 nM, about 1 nM to about 3.8 nM, about 1 nM to about 3.7 nM, about 1 nM to about 3.6 nM, about 1 nM to about 3.5 nM, about 1 nM to about 3.4 nM, about 1 nM to about 3.3 nM, about 1 nM to about 3.2 nM, about 1 nM to about 3.1 nM, about 1 nM to about 3 nM, about 1 nM to about 2.9 nM, about 1 nM to about 2.8 nM, about 1 nM to about 2.7 nM, about 1 nM to about 2.6 nM, about 1 nM to about 2.5 nM, about 1 nM to about 2.4 nM, about 1 nM to about 2.3 nM, about 1 nM to about 2.2 nM, about 1 nM to about 2.1 nM, about 1 nM to about 2 nM, about 1 nM to about 1.9 nM, about 1 nM to about 1.8 nM, about 1 nM to about 1.7 nM, about 1 nM to about 1.6 nM, or about 1 nM to about 1.5 nM of a first polypeptide comprising B7-H6 or a functional fragment thereof.

In some embodiments, the liquid culture medium includes about 0.05 nM, about 0.1 nM, about 0.15 nM, about 0.2 nM, about 0.25 nM, about 0.3 nM, about 0.35 nM, about 0.4 nM, about nM, about 0.5 nM, about 0.55 nM, about 0.6 nM, about 0.65 nM, about 0.7 nM, about 0.75 nM, about 0.8 nM, about 0.85 nM, about 0.9 nM, about 0.95 nM, about 1 nM, about 1.05 nM, about 1.1 nM, about 1.15 nM, about 1.2 nM, about 1.25 nM, about 1.3 nM, about 1.35 nM, about 1.4 nM, about 1.45 nM, about 1.5 nM, about 1.55 nM, about 1.6 nM, about 1.65 nM, about 1.7 nM, about 1.75 nM, about 1.8 nM, about 1.85 nM, about 1.9 nM, about 1.95 nM, about 2 nM, about 2.05 nM, about 2.1 nM, about 2.15 nM, about 2.2 nM, about 2.25 nM, about 2.3 nM, about 2.35 nM, about 2.4 nM, about 2.45 nM, about 2.5 nM, about 2.55 nM, about 2.6 nM, about 2.65 nM, about 2.7 nM, about 2.75 nM, about 2.8 nM, about 2.85 nM, about 2.9 nM, about 2.95 nM, about 3 nM, about 3.05 nM, about 3.1 nM, about 3.15 nM, about 3.2 nM, about 3.25 nM, about 3.3 nM, about 3.35 nM, about 3.4 nM, about 3.45 nM, about 3.5 nM, about 3.55 nM, about 3.6 nM, about 3.65 nM, about 3.7 nM, about 3.75 nM, about 3.8 nM, about 3.85 nM, about 3.9 nM, about 3.95 nM, about 4 nM, about 0.1 nM to about 4 nM, about 0.1 nM to about 3.9 nM, about nM to about 3.8 nM, about 0.1 nM to about 3.7 nM, about 0.1 nM to about 3.6 nM, about 0.1 nM to about 3.5 nM, about 0.1 nM to about 3.4 nM, about 0.1 nM to about 3.3 nM, about 0.1 nM to about 3.2 nM, about 0.1 nM to about 3.1 nM, about 0.1 nM to about 3 nM, about 0.1 nM to about 2.9 nM, about 0.1 nM to about 2.8 nM, about 0.1 nM to about 2.7 nM, about 0.1 nM to about 2.6 nM, about 0.1 nM to about 2.5 nM, about 0.1 nM to about 2.4 nM, about 0.1 nM to about 2.3 nM, about 0.1 nM to about 2.2 nM, about 0.1 nM to about 2.1 nM, about 0.1 nM to about 2 nM, about 0.1 nM to about 1.9 nM, about 0.1 nM to about 1.8 nM, about 0.1 nM to about 1.7 nM, about 0.1 nM to about 1.6 nM, about 0.1 nM to about 1.5 nM, about 0.5 nM to about 4 nM, about 0.5 nM to about 3.9 nM, about 0.5 nM to about 3.8 nM, about 0.5 nM to about 3.7 nM, about 0.5 nM to about 3.6 nM, about 0.5 nM to about 3.5 nM, about 0.5 nM to about 3.4 nM, about 0.5 nM to about 3.3 nM, about 0.5 nM to about 3.2 nM, about 0.5 nM to about 3.1 nM, about 0.5 nM to about 3 nM, about 0.5 nM to about 2.9 nM, about 0.5 nM to about 2.8 nM, about 0.5 nM to about 2.7 nM, about 0.5 nM to about 2.6 nM, about 0.5 nM to about 2.5 nM, about 0.5 nM to about 2.4 nM, about 0.5 nM to about 2.3 nM, about 0.5 nM to about 2.2 nM, about 0.5 nM to about 2.1 nM, about 0.5 nM to about 2 nM, about 0.5 nM to about 1.9 nM, about nM to about 1.8 nM, about 0.5 nM to about 1.7 nM, about 0.5 nM to about 1.6 nM, about 0.5 nM to about 1.5 nM, about 1 nM to about 4 nM, about 1 nM to about 3.9 nM, about 1 nM to about 3.8 nM, about 1 nM to about 3.7 nM, about 1 nM to about 3.6 nM, about 1 nM to about 3.5 nM, about 1 nM to about 3.4 nM, about 1 nM to about 3.3 nM, about 1 nM to about 3.2 nM, about 1 nM to about 3.1 nM, about 1 nM to about 3 nM, about 1 nM to about 2.9 nM, about 1 nM to about 2.8 nM, about 1 nM to about 2.7 nM, about 1 nM to about 2.6 nM, about 1 nM to about 2.5 nM, about 1 nM to about 2.4 nM, about 1 nM to about 2.3 nM, about 1 nM to about 2.2 nM, about 1 nM to about 2.1 nM, about 1 nM to about 2 nM, about 1 nM to about 1.9 nM, about 1 nM to about 1.8 nM, about 1 nM to about 1.7 nM, about 1 nM to about 1.6 nM, or about 1 nM to about 1.5 nM of a second polypeptide comprising 4-1BBL or a functional fragment thereof.

In some embodiments, the liquid culture medium includes about 0.05 nM, about 0.1 nM, about 0.15 nM, about 0.2 nM, about 0.25 nM, about 0.3 nM, about 0.35 nM, about 0.4 nM, about nM, about 0.5 nM, about 0.55 nM, about 0.6 nM, about 0.65 nM, about 0.7 nM, about 0.75 nM, about 0.8 nM, about 0.85 nM, about 0.9 nM, about 0.95 nM, about 1 nM, about 1.05 nM, about 1.1 nM, about 1.15 nM, about 1.2 nM, about 1.25 nM, about 1.3 nM, about 1.35 nM, about 1.4 nM, about 1.45 nM, about 1.5 nM, about 1.55 nM, about 1.6 nM, about 1.65 nM, about 1.7 nM, about 1.75 nM, about 1.8 nM, about 1.85 nM, about 1.9 nM, about 1.95 nM, about 2 nM, about 2.05 nM, about 2.1 nM, about 2.15 nM, about 2.2 nM, about 2.25 nM, about 2.3 nM, about 2.35 nM, about 2.4 nM, about 2.45 nM, about 2.5 nM, about 2.55 nM, about 2.6 nM, about 2.65 nM, about 2.7 nM, about 2.75 nM, about 2.8 nM, about 2.85 nM, about 2.9 nM, about 2.95 nM, about 3 nM, about 3.05 nM, about 3.1 nM, about 3.15 nM, about 3.2 nM, about 3.25 nM, about 3.3 nM, about 3.35 nM, about 3.4 nM, about 3.45 nM, about 3.5 nM, about 3.55 nM, about 3.6 nM, about 3.65 nM, about 3.7 nM, about 3.75 nM, about 3.8 nM, about 3.85 nM, about 3.9 nM, about 3.95 nM, about 4 nM, about 0.1 nM to about 4 nM, about 0.1 nM to about 3.9 nM, about nM to about 3.8 nM, about 0.1 nM to about 3.7 nM, about 0.1 nM to about 3.6 nM, about 0.1 nM to about 3.5 nM, about 0.1 nM to about 3.4 nM, about 0.1 nM to about 3.3 nM, about 0.1 nM to about 3.2 nM, about 0.1 nM to about 3.1 nM, about 0.1 nM to about 3 nM, about 0.1 nM to about 2.9 nM, about 0.1 nM to about 2.8 nM, about 0.1 nM to about 2.7 nM, about 0.1 nM to about 2.6 nM, about 0.1 nM to about 2.5 nM, about 0.1 nM to about 2.4 nM, about 0.1 nM to about 2.3 nM, about 0.1 nM to about 2.2 nM, about 0.1 nM to about 2.1 nM, about 0.1 nM to about 2 nM, about 0.1 nM to about 1.9 nM, about 0.1 nM to about 1.8 nM, about 0.1 nM to about 1.7 nM, about 0.1 nM to about 1.6 nM, about 0.1 nM to about 1.5 nM, about 0.5 nM to about 4 nM, about 0.5 nM to about 3.9 nM, about 0.5 nM to about 3.8 nM, about 0.5 nM to about 3.7 nM, about 0.5 nM to about 3.6 nM, about 0.5 nM to about 3.5 nM, about 0.5 nM to about 3.4 nM, about 0.5 nM to about 3.3 nM, about 0.5 nM to about 3.2 nM, about 0.5 nM to about 3.1 nM, about 0.5 nM to about 3 nM, about 0.5 nM to about 2.9 nM, about 0.5 nM to about 2.8 nM, about 0.5 nM to about 2.7 nM, about 0.5 nM to about 2.6 nM, about 0.5 nM to about 2.5 nM, about 0.5 nM to about 2.4 nM, about 0.5 nM to about 2.3 nM, about 0.5 nM to about 2.2 nM, about 0.5 nM to about 2.1 nM, about 0.5 nM to about 2 nM, about 0.5 nM to about 1.9 nM, about nM to about 1.8 nM, about 0.5 nM to about 1.7 nM, about 0.5 nM to about 1.6 nM, about 0.5 nM to about 1.5 nM, about 1 nM to about 4 nM, about 1 nM to about 3.9 nM, about 1 nM to about 3.8 nM, about 1 nM to about 3.7 nM, about 1 nM to about 3.6 nM, about 1 nM to about 3.5 nM, about 1 nM to about 3.4 nM, about 1 nM to about 3.3 nM, about 1 nM to about 3.2 nM, about 1 nM to about 3.1 nM, about 1 nM to about 3 nM, about 1 nM to about 2.9 nM, about 1 nM to about 2.8 nM, about 1 nM to about 2.7 nM, about 1 nM to about 2.6 nM, about 1 nM to about 2.5 nM, about 1 nM to about 2.4 nM, about 1 nM to about 2.3 nM, about 1 nM to about 2.2 nM, about 1 nM to about 2.1 nM, about 1 nM to about 2 nM, about 1 nM to about 1.9 nM, about 1 nM to about 1.8 nM, about 1 nM to about 1.7 nM, about 1 nM to about 1.6 nM, or about 1 nM to about 1.5 nM of a fourth polypeptide comprising IL-21 or a functional fragment thereof.

In some embodiments, the liquid culture medium includes about 0.05 nM, about 0.1 nM, about 0.15 nM, about 0.2 nM, about 0.25 nM, about 0.3 nM, about 0.35 nM, about 0.4 nM, about nM, about 0.5 nM, about 0.55 nM, about 0.6 nM, about 0.65 nM, about 0.7 nM, about 0.75 nM, about 0.8 nM, about 0.85 nM, about 0.9 nM, about 0.95 nM, about 1 nM, about 1.05 nM, about 1.1 nM, about 1.15 nM, about 1.2 nM, about 1.25 nM, about 1.3 nM, about 1.35 nM, about 1.4 nM, about 1.45 nM, about 1.5 nM, about 1.55 nM, about 1.6 nM, about 1.65 nM, about 1.7 nM, about 1.75 nM, about 1.8 nM, about 1.85 nM, about 1.9 nM, about 1.95 nM, about 2 nM, about 2.05 nM, about 2.1 nM, about 2.15 nM, about 2.2 nM, about 2.25 nM, about 2.3 nM, about 2.35 nM, about 2.4 nM, about 2.45 nM, about 2.5 nM, about 2.55 nM, about 2.6 nM, about 2.65 nM, about 2.7 nM, about 2.75 nM, about 2.8 nM, about 2.85 nM, about 2.9 nM, about 2.95 nM, about 3 nM, about 3.05 nM, about 3.1 nM, about 3.15 nM, about 3.2 nM, about 3.25 nM, about 3.3 nM, about 3.35 nM, about 3.4 nM, about 3.45 nM, about 3.5 nM, about 3.55 nM, about 3.6 nM, about 3.65 nM, about 3.7 nM, about 3.75 nM, about 3.8 nM, about 3.85 nM, about 3.9 nM, about 3.95 nM, about 4 nM, about 0.1 nM to about 4 nM, about 0.1 nM to about 3.9 nM, about nM to about 3.8 nM, about 0.1 nM to about 3.7 nM, about 0.1 nM to about 3.6 nM, about 0.1 nM to about 3.5 nM, about 0.1 nM to about 3.4 nM, about 0.1 nM to about 3.3 nM, about 0.1 nM to about 3.2 nM, about 0.1 nM to about 3.1 nM, about 0.1 nM to about 3 nM, about 0.1 nM to about 2.9 nM, about 0.1 nM to about 2.8 nM, about 0.1 nM to about 2.7 nM, about 0.1 nM to about 2.6 nM, about 0.1 nM to about 2.5 nM, about 0.1 nM to about 2.4 nM, about 0.1 nM to about 2.3 nM, about 0.1 nM to about 2.2 nM, about 0.1 nM to about 2.1 nM, about 0.1 nM to about 2 nM, about 0.1 nM to about 1.9 nM, about 0.1 nM to about 1.8 nM, about 0.1 nM to about 1.7 nM, about 0.1 nM to about 1.6 nM, about 0.1 nM to about 1.5 nM, about 0.5 nM to about 4 nM, about 0.5 nM to about 3.9 nM, about 0.5 nM to about 3.8 nM, about 0.5 nM to about 3.7 nM, about 0.5 nM to about 3.6 nM, about 0.5 nM to about 3.5 nM, about 0.5 nM to about 3.4 nM, about 0.5 nM to about 3.3 nM, about 0.5 nM to about 3.2 nM, about 0.5 nM to about 3.1 nM, about 0.5 nM to about 3 nM, about 0.5 nM to about 2.9 nM, about 0.5 nM to about 2.8 nM, about 0.5 nM to about 2.7 nM, about 0.5 nM to about 2.6 nM, about 0.5 nM to about 2.5 nM, about 0.5 nM to about 2.4 nM, about 0.5 nM to about 2.3 nM, about 0.5 nM to about 2.2 nM, about 0.5 nM to about 2.1 nM, about 0.5 nM to about 2 nM, about 0.5 nM to about 1.9 nM, about nM to about 1.8 nM, about 0.5 nM to about 1.7 nM, about 0.5 nM to about 1.6 nM, about 0.5 nM to about 1.5 nM, about 1 nM to about 4 nM, about 1 nM to about 3.9 nM, about 1 nM to about 3.8 nM, about 1 nM to about 3.7 nM, about 1 nM to about 3.6 nM, about 1 nM to about 3.5 nM, about 1 nM to about 3.4 nM, about 1 nM to about 3.3 nM, about 1 nM to about 3.2 nM, about 1 nM to about 3.1 nM, about 1 nM to about 3 nM, about 1 nM to about 2.9 nM, about 1 nM to about 2.8 nM, about 1 nM to about 2.7 nM, about 1 nM to about 2.6 nM, about 1 nM to about 2.5 nM, about 1 nM to about 2.4 nM, about 1 nM to about 2.3 nM, about 1 nM to about 2.2 nM, about 1 nM to about 2.1 nM, about 1 nM to about 2 nM, about 1 nM to about 1.9 nM, about 1 nM to about 1.8 nM, about 1 nM to about 1.7 nM, about 1 nM to about 1.6 nM, or about 1 nM to about 1.5 nM of a third polypeptide comprising ICAM-1 or a functional fragment thereof.

In some embodiments of any of the methods described herein, the methods comprise contacting the NK cell with an anti-CD16 antibody or antigen-binding fragment thereof. In some embodiments, a liquid culture medium provided herein includes an anti-CD16 antibody or antigen-binding fragment thereof. In some embodiments, the liquid culture medium includes about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, about 20 μg/mL, about 21 μg/mL, about 22 μg/mL, about 23 μg/mL, about 24 μg/mL, about 25 μg/mL, about 26 μg/mL, about 27 μg/mL, about 28 μg/mL, about 29 μg/mL, about 30 μg/mL, about 1 μg/mL to about 30 μg/mL, about 1 μg/mL to about 25 μg/mL, about 1 μg/mL to about 20 μg/mL, about 1 μg/mL to about 15 μg/mL, about 1 μg/mL to about 10 μg/mL, about 1 μg/mL to about 5 μg/mL, about 5 μg/mL to about 30 μg/mL, about 5 μg/mL to about 25 μg/mL, about 5 μg/mL to about 20 μg/mL, about 5 μg/mL to about 15 μg/mL, or about 5 μg/mL to about 10 μg/mL, of an anti-CD16 antibody or an antigen-binding fragment thereof. Non-limiting examples of anti-CD16 antibodies are known in the art (e.g., anti-human CD16 monoclonal antibody, clone 3G8 (Cat. No. 302001, Biolegend), eBioCB16 (CB16) monoclonal antibody (eBioscience™); MEM-154 mouse monoclonal anti-CD16 antibody (ab46679; Abcam™); and rabbit polyclonal anti-CD16 antibody (ab203883; Abcam™)), and anti-human CD16 monoclonal antibody, clone GRM1 (Product No. C465, Leinco Technologies, Inc.). The anti-CD16 antibody or antigen-binding fragment thereof may be an antibody (e.g., IgG1, IgG2, IgG3, IgG4), a VHH, an scFv, a Fab′ fragment, an Fv fragment, or a F(ab′)2 fragment.

In some embodiments, the liquid culture medium includes a polypeptide comprising IL-21 or a functional fragment thereof. IL-21 is a cytokine that has potent regulatory effects on cells of the immune system. In some embodiments, the liquid culture medium does not include a polypeptide comprising IL-21 or a functional fragment thereof. In some embodiments, the polypeptide comprising IL-21 or a functional fragment thereof includes an amino acid sequence that is at least 80%, at least 90%, at least 95%, at least 96%, at least 97%, at least 98%, at least 99%, or 100% identical to SEQ ID NO: 23, 24, or 25.

In some embodiments, the liquid culture medium does not include IL-12 or a functional fragment thereof.

In some embodiments, the method includes replenishing the liquid culture medium one or more times. For example, the liquid culture medium is replenished 2, 3, 4, 5, 6, 7, 8, 9, 10, 12, 14, 16, 18, 20, 22, 24, 26, 28, 30 or more times. In some embodiments, replenishing the liquid culture medium is performed using batch fed culturing. In some embodiments, replenishing the liquid culture medium is performed using perfusion culturing. In some embodiments, replenishing is performed by replacement of the liquid culture medium. In some embodiments, replenishing the liquid culture medium occurs about every day, about every two days, about every 3 days, about every four days, about every five days, about every six days, about every seven days, about every eight days, about every nine days, about every ten days, about every eleven days, or about every twelve days. In some embodiments, replenishing the liquid culture medium occurs about every day. In some embodiments, replenishing the liquid culture medium occurs about every one or two days.

In some embodiments, one or more of the first polypeptide, the second polypeptide, and the third polypeptide are directly or indirectly attached to one or more substrate(s). In some embodiments, two or more of the first polypeptide, the second polypeptide, and the third polypeptide are directly or indirectly attached to one or more substrate(s). In some embodiments, the first polypeptide, the second polypeptide, and the third polypeptide are directly or indirectly attached to one or more substrate(s).

In some embodiments, one or more of the first polypeptide, the second polypeptide, and the third polypeptide are directly attached to the one or more substrate(s). In some embodiments, two or more of the first polypeptide, the second polypeptide, and the third polypeptide are directly attached to the one or more substrate(s). In some embodiments, the first polypeptide, the second polypeptide, and the third polypeptide are directly attached to the one or more substrate(s).

In some embodiments, one or more of the first polypeptide, the second polypeptide, and the third polypeptide are indirectly attached to the one or more substrate(s). In some embodiments, two or more of the first polypeptide, the second polypeptide, and the third polypeptide are indirectly attached to one or more substrate(s). In some embodiments, the first polypeptide, the second polypeptide, and the third polypeptide are indirectly attached to the one or more substrate(s).

In some embodiments, one or more of the first polypeptide, the second polypeptide, and the third polypeptide is attached (e.g., directly or indirectly) to the same substrate (e.g., bead or flask). In some embodiments, one or more of the first polypeptide, the second polypeptide and the third polypeptide is attached to two or more separate substrates (e.g., bead or flask). In some embodiments, the first polypeptide is attached to a first substrate, the second polypeptide is attached to a second substrate, and the third polypeptide is attached to a third substrate. In some embodiments, the first polypeptide and the second polypeptide are attached to a first substrate and the third polypeptide is attached to a second substrate. In some embodiments, the first polypeptide is attached to a first substrate, and the second polypeptide and the third polypeptide are attached to a second substrate. In some embodiments, one or more of the first polypeptide, the second polypeptide and the third polypeptide is attached to a substrate (e.g., the same or different substrates), and one or more of the first polypeptide, the second polypeptide, the third polypeptide is in solution (e.g., in a cell culture medium).

In some embodiments, one or more of the first polypeptide, the second polypeptide, and the third polypeptide includes a tag. In some embodiments, two or more of the first polypeptide, the second polypeptide, and the third polypeptide include a tag. In some embodiments, the first polypeptide, the second polypeptide, and the third polypeptide include a tag. In some embodiments, the one or more substrate(s) includes an agent that specifically binds to the tag. In some embodiments, the tag is an Fc domain, a myc tag, a HA tag, or a GST tag. In some embodiments, the tag is an Fc domain, and the agent that specifically binds to the tag is an anti-Fc antibody or antigen-binding fragment thereof. In some embodiments, the tag is a myc tag and the agent is an anti-myc tag antibody or antigen-binding fragment thereof. In some embodiments, the tag is an HA tag and the agent that specifically binds to the tag is an anti-HA tag antibody or antigen-binding fragment thereof. In some embodiments, the tag is a GST tag and the agent that specifically binds to the tag is an anti-GST tag antibody or antigen-binding fragment thereof.

In some embodiments, one or more of the first polypeptide, the second polypeptide, and the third polypeptide, and the fourth polypeptide, further comprises a linker. For example, linkers can be used to attach the first polypeptide, the second polypeptide, the third polypeptide, and the fourth polypeptide, to one or more substrate(s) described herein. For example, one or more of the first polypeptide, the second polypeptide, the third polypeptide, and the fourth polypeptide may include an N-terminal linker and/or a C-terminal linker (e.g., which may be disposed between the substrate and the other portions of the first polypeptide, the second polypeptide, the third polypeptide, and/or the fourth polypeptide when attached to the substrate)). Linkers of varying lengths can be important in reducing steric hindrance, improving protein folding, and/or protein stability which can increase the potency of one or more of the polypeptides described herein, particularly in the context of certain substrates, such as beads. In some embodiments, the linker is an amino acid repeat, such as a glycine-serine (GS) repeat. In some embodiment, the GS repeat is about 2, about 4, about 6, about 8, about 10, about 12, about 14, about 16, about 18, about 20, about 22, about 24, about 26, about 28, about 30, about 32, about 34, about 36, about 38, about 40, about 42, about 44, about 46, about 48, about 50, or more amino acid residues. Additionally and/or alternatively, the linker can include an IgG hinge regions. In some embodiments, the IgG hinge region is an IgG1 hinge region. In some embodiments, the hinge region is an IgG2 hinge region. In some embodiments, the IgG hinge region is an IgG3 hinge region. In some embodiments, the IgG hinge region is an IgG4 hinge region.

In some embodiments, one or more of the first polypeptide, the second polypeptide, the third polypeptide, and the fourth polypeptide are attached to the surface of the one or more substrate(s). In some embodiments, the one or more substrate(s) (e.g., a bead or flask) contains one or more materials at or near its surface that can be used to attach (e.g., couple, link, or conjugate) one or more polypeptides described herein (e.g., a first polypeptide, a second polypeptide, a third polypeptide, and/or a fourth polypeptide) to the one or more substrate(s). In some embodiments, surface of the one or more substrate(s) (e.g., a bead or flask) is functionalized (e.g., includes functional groups that are capable of forming a covalent bond with a molecule (e.g., a polypeptide). In some embodiments, the one or more substrate(s) (e.g., a bead or flask) comprises surface exposed carboxyl, amino, tosyl, hydroxyl, epoxy, iodoacetyl and/or chloromethyl groups. In some embodiments the one or more substrate(s) includes surface-exposed agarose. In some embodiments, the one or more substrate(s) includes attached reagents that can bind or attach polypeptides. In some embodiment, the one or more substrate(s) includes surface exposed protein A, protein G, biotin, and/or an antibody or antigen-binding fragment thereof (e.g., an anti-Fc antibody or antigen-binding fragment thereof (e.g., a rabbit anti-human Fc antibody or antigen-binding fragment thereof), an anti-myc tag antibody or antigen-binding fragment thereof, an anti-HA tag antibody or antigen-binding fragment thereof, or an anti-GST tag antibody or antigen-binding fragment thereof). Methods for attaching (e.g., conjugating) polypeptides to a substrate are known in the art, including chemical conjugation (e.g., click chemistry) and can be used to attach polypeptides to the one or more substrate(s).

In some embodiments, the one or more substrate(s) is a bead. In some embodiments, the bead has a diameter of from about 10 nm to about 1,000 nm, from about 50 nm to about 1,000 nm, from about 100 nm to about 1,000 nm, from about 200 nm to about 1,000 nm, from about 300 nm to about 1,000 nm, from about 400 nm to about 1,000 nm, from about 500 nm to about 1,000 nm, from about 600 nm to about 1,000 nm, from about 700 nm to about 1,000 nm, from about 800 nm to about 1000 nm, from about 900 nm to about 1,000 nm, from about 10 nm to about 750 nm, from about 50 nm to about 750 nm, from about 100 nm to about 750 nm, from about 200 nm to about 750 nm, from about 300 nm to about 750 nm, from about 400 nm to about 750 nm, from about 500 nm to about 7500 nm, from about 600 nm to about 750 nm, from about nm to about 500 nm, from about 50 nm to about 500 nm, from about 100 nm to about 500 nm, from about 200 nm to about 500 nm, from about 300 nm to about 500 nm, from about 400 nm to about 500 nm, from about 10 nm to about 250 nm, from about 50 nm to about 250 nm, from about 100 nm to about 250 nm, from about 200 nm to about 250 nm, from about 10 nm to about 100 nm, from about 20 nm to about 100 nm, from about 30 nm to about 100 nm, or from about nm to about 100 nm, from about 50 nm to about 100 nm, from about 60 nm to about 100 nm, from about 70 nm to about 100 nm, from about 80 nm to about 100 nm, or from about 90 nm to 100 nm. In some embodiments, the bead has a diameter of about 10 nm, 20 nm, 30 nm, 40 nm, nm, 60 nm, 70 nm, 80 nm, 90 nm, 100 nm, 150 nm, 200 nm, 300 nm, 400 nm, 500 nm, 600 nm, 700 nm, 800 nm, or 900 nm. In some embodiments, the bead has a diameter of from about 1.0 μm to about 10.0 μm, from about 2.0 μm to about 10.0 μm, from about 3.0 μm to about 10.0 μm, from about 4.0 μm to about 10.0 μm, from about 1.0 μm to about 9.0 μm, from about 2.0 μm to about 9.0 μm, from about 3.0 μm to about 9.0 μm, from about 4.0 μm to about 9.0 μm, from about 1.0 μm to about 8.0 μm, from about 2.0 μm to about 8.0 μm, from about 3.0 μm to about 8.0 μm, from about 4.0 μm to about 8.0 μm, from about 1.0 μm to about 7.0 μm, from about 2.0 μm to about 7.0 μm, from about 3.0 μm to about 7.0 μm, from about 4.0 μm to about 7.0 μm, from about 1.0 μm to about 6.0 μm, from about 2.0 μm to about 6.0 μm, from about 3.0 μm to about 6.0 μm, from about 4.0 μm to about 6.0 μm, from about 1.0 μm to about 5.0 μm, from about 2.0 μm to about 5.0 μm, from about 3.0 μm to about 5.0 μm, or from about 4.0 μm to about 5.0 μm. In some embodiments, the bead has a diameter of 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm or 10 μm.

In some embodiments, the one or more substrate(s) (e.g., bead or flask) includes a hydrogel. In some embodiments, the one or more substrate(s) (e.g., bead or flask) includes a natural polymer. In some embodiments, the one or more substrate(s) (e.g., bead or flask) includes a synthetic polymer. In some embodiments, the one or more substrate(s) (e.g., bead or flask) includes a co-polymer, where the co-polymer is a co-polymer of natural polymers. In some embodiments, the one or more substrate(s) (e.g., bead or flask) includes a co-polymer, where the co-polymer is a co-polymer of synthetic polymers. In some embodiments, the one or more substrate(s) (e.g., bead or flask) includes a co-polymer, wherein the co-polymer is a co-polymer of a natural polymer and a synthetic polymer. Natural polymers are known in the art. Non-limiting examples of natural polymers include: alginate, agarose, carrageenan, chitosan, dextran, carboxymethylcellulose, heparin, hyaluronic acid, polyamino acids, collagen, gelatin, fibrin, fibrous protein-based biopolymers. Synthetic polymers are known in the art. Non-limiting examples of synthetic polymers include: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene).

In some embodiments, the one or more substrate(s) is a bead capable of reacting with a magnetic field. In some embodiments, the bead is a magnetic bead (e.g., paramagnetic, superparamagnetic, ferromagnetic or ferrimagnetic). In some embodiments, the one or more substrate(s) is a bead that does not display magnetic properties unless exposed to a magnetic field. In some embodiments, the one or more substrate(s) is a bead comprising a magnetic core, a paramagnetic core, or a superparamagnetic core. In some embodiments, the bead core contains a metal (e.g., iron, nickel, copper, cobalt, gadolinium, manganese, tantalum, zinc, zirconium, or any combination thereof). In some embodiments, the bead core contains metal oxides (e.g., iron oxide or chromium dioxide), ferrites (e.g., manganese ferrites, cobalt ferrites, and nickel ferrites), hematite, or metal alloys. In some embodiments, the bead core contains magnetite, maghemite or greigite. In some embodiments, the one or more substrate(s) is a bed that contains a magnetic, paramagnetic, and/or superparamagnetic core covered by a coating (e.g., a surface functionalized coating). In some embodiments, the coating includes one or more of a polymer (e.g., polyethylene glycol, poly (lactic-co-glycolic acid), polyglutaraldehyde, polyurethane, polystyrene, or a polyvinyl alcohol), a polysaccharide, a silica, a fatty acid, a polypeptide, a carbon, agarose, and a Sepharose® resin).

In some embodiments, the one or more substrate(s) is a culture vessel (e.g., a bioreactor). For example, the culture vessel can be a cell culture flask (e.g., a multilayer cell culture flask), cell culture bag, tube (e.g., a roller bottle), plate (e.g., a multiwell plate), dish, or well (e.g., a round-bottomed well or a flat-bottomed well) in a multi-well plate). In some embodiments, two or more culture vessels can be used when performing the methods described herein. For example, during expansion, the NK cells can be transferred to a second, a third, a fourth, a fifth, a sixth, a seventh, an eight, a ninth, or a tenth culture vessel. In some embodiments, each of the culture vessels used to perform the methods are the same. In some embodiments, the culture vessels used to perform the methods are different.

In some embodiments, the one or more substrate(s) can form part of a culture vessel. In some embodiments the culture vessel is a large surface area cell culture flask or plate (e.g., a T-flask (e.g., T25, T75, T125, T175, and T250 flasks), Nunc® TripleFlask™ cell culture vessels (Thermo Fisher Scientific), a HYPERFlask® cell culture vessels (Corning®), HYPERStack® cell culture vessels (Corning), CellCube® modules (Corning), and Nunc Cell Factory culture systems). In some embodiments, the culture vessels may be made of any suitable gas-permeable/liquid impermeable materials, including but not limited to, polystyrene, polycarbonate, ethylene vinyl acetate, polysulfone, polymethyl pentene (PMP), polytetrafluoroethylene (PTFE) or compatible fluoropolymer, a silicone rubber or copolymer, poly(styrene-butadiene-styrene), and polyolefin (e.g., polyethylene or polypropylene), and combinations of these materials. In some embodiments, the culture vessel is coated with a cell adhesion substance (e.g., extracellular matrix component(s) to improve the adhesiveness of the vessel surface to cells (e.g., NK cells)). Non-limiting cell adhesion substances include collagen, gelatin, poly-L-lysine, poly-D-lysine, poly-L-ornithine, laminin, vitronectin, chondronectin, and fibronectin, and any combination of the foregoing including fragments thereof.

In some embodiments, more than one and/or different culture vessels are used to perform the methods described herein.

In some embodiments, the first polypeptide, the second polypeptide, and the third polypeptide are present in approximately equal amounts or concentrations (e.g., in the liquid culture medium or attached to one or more substrate(s) described herein).

In some embodiments, contacting is performed in the presence of a liquid culture medium (e.g., any of the liquid culture media described herein).

In some embodiments of any of the methods described herein, the NK cell is grown in adhesion culture. In some embodiments of any of the methods described herein, the NK cell is grown in suspension culture.

In some embodiments, the method further includes, before the contacting step, culturing the NK cell in the presence of a feeder cell. In some embodiments, the method further includes after the contacting step, culturing the NK cell in the presence of a feeder cell. In some embodiments, the feeder cell is an irradiated feeder cell (e.g., irradiated with gamma rays in the range of from about 6,000 to 10,000 rads). In some embodiments, the feeder cell has been engineered to express one or more proteins (e.g., cytokines such as IL-21 or a functional fragment thereof and IL-15 or a functional fragment thereof). In some embodiments, the feeder cell is a Jurkat cell, an Epstein-Barr virus- (EBV-) immortalized lymphoblastoid cell line (LCLs), a K562 cell, a T-cell (e.g., a CD4⁺ T cell or a CD8⁺ T cell), a HuT78 cell, and/or a peripheral blood mononuclear cell (PBMC).

In some embodiments, the method further includes after the contacting step, culturing the NK cell in a liquid culture medium not including each of (i) a first polypeptide comprising B7-H6 or a functional fragment thereof, or an anti-NKp30 antigen-binding domain, (ii) a second polypeptide comprising 4-1BBL or a functional fragment thereof, and (iii) a third polypeptide comprising ICAM-1 or a functional fragment thereof. In some embodiments, the method further includes after the contacting step, culturing the NK cell in a liquid culture medium not including a first polypeptide comprising B7-H6 or a functional fragment thereof, or an anti-NKp30 antigen-binding domain. In some embodiments, the method further includes after the contacting step, culturing the NK cell in a liquid culture medium not including a second polypeptide comprising 4-1BBL or a functional fragment thereof. In some embodiments, the method further includes after the contacting step, culturing the NK cell in a liquid culture medium not including a third polypeptide comprising ICAM-1 or a functional fragment thereof. In some embodiments, the method further includes after the contacting step, culturing the NK cell in a liquid culture medium not including a fourth polypeptide including IL-21 or a functional fragment thereof.

In some embodiments, the method further includes, before the contacting step, introducing into the NK cell a nucleic acid encoding one or more exogenous polypeptides. In some embodiments, the method further includes, after the contacting step, introducing into the NK cell a nucleic acid encoding one or more exogenous polypeptides. The nucleic acid can be introduced into the NK cell using any suitable means including transfection, transduction (e.g., using a retrovirus, adenovirus or lentivirus), using a transposon system, and electroporation. In some embodiments, the one or more exogenous polypeptides is a chimeric antigen receptor (CAR). In some embodiments, the one or more exogenous polypeptides is a therapeutic protein. In some embodiments, the NK cell is a genetically engineered NK cell.

Also provided herein are methods of expanding an NK cell including contacting an NK cell disposed in a liquid culture medium, in the absence of a feeder cell, with (i) a first polypeptide including B7-H6 or a functional fragment thereof, or an anti-NKp30 antigen-binding domain, (ii) a second polypeptide including 4-1BBL or a functional fragment thereof, (iii) a third polypeptide including ICAM-1 or a functional fragment thereof, and (iv) a fourth polypeptide including IL-21 or a functional fragment thereof, under conditions sufficient for expansion of the NK cell.

In some embodiments, the NK cell is periodically re-contacted with the first polypeptide, the second polypeptide, the third polypeptide, and the fourth polypeptide.

In some embodiments, the methods provided herein are performed for about 1, about 5, about 10, about 15, about 20, about 25, about 30, about 35, about 40, about 45, about 50, about or about 60 days. In some embodiments, the methods provided herein are performed for about 1 day to about 60 days (e.g., or any of the subranges of this range described herein).

In some embodiments, the NK cell is re-contacted with the first polypeptide, the second polypeptide, the third polypeptide, and the fourth polypeptide at about day 1, day 2, day 3, day 4, day 5, day 10, day 15, day 20, day 25, day 30, day 35, day 40, day 45, day 50, day 55, and/or at about day 60 after an initial contacting (e.g., after an initial contacting step). In some embodiments, the NK cell is re-contacted with the first polypeptide, the second polypeptide, the third polypeptide, and the fourth polypeptide at about day 1, day 2, day 3, day 4, day 5, day 6, day 9, day 12, day 15, day 18, day 21, day 24, day 27, day 30, day 33, day 36, day 39, day 42, day 45, day 48, day 51, day 54, day 57, and/or day 60 after an initial contacting (e.g., after an initial contacting step). In some embodiments, the NK cell is re-contacted with the first polypeptide, the second polypeptide, the third polypeptide, and the fourth polypeptide at about day 1, day 2, day 3, day 4, day 5, day 8, day 12, day 16, day 20, day 24, day 28, day 32, day 36, day 40, day 44, day 48, day 52, day 56, and/or day 60 after an initial contacting (e.g., after an initial contacting step). In some embodiments, the NK cell is re-contacted with the first polypeptide, the second polypeptide, the third polypeptide, and the fourth polypeptide at about day 1, day 2, day 3, day 4, day 5, day 6, day 12, day 18, day 24, day 30, day 36, day 42, day 48, day 54, and/or day 60 after an initial contacting (e.g., after an initial contacting step). In some embodiments, the NK cell is re-contacted with the first polypeptide, the second polypeptide, the third polypeptide, and the fourth polypeptide at about day 1, day 2, day 3, day 4, day 5, day 7, day 14, day 21, day 28, day 35, day 42, day 49, and/or day 56 after an initial contacting (e.g., after an initial contacting step). In some embodiments, the NK cell is re-contacted with the first polypeptide, the second polypeptide, the third polypeptide, and the fourth polypeptide at about day 1, day 2, day 3, day 4, day 5, day 8, day 16, day 24, day 32, day 40, day 48, and/or day 56 after an initial contacting (e.g., after an initial contacting step). In some embodiments, the NK cell is re-contacted with the first polypeptide, the second polypeptide, the third polypeptide, and the fourth polypeptide at about day 1, day 2, day 3, day 4, day 5, day 9, day 18, day 27, day 36, day 45, and/or day 54 after an initial contacting (e.g., after an initial contacting step). In some embodiments, the NK cell is re-contacted with the first polypeptide, the second polypeptide, the third polypeptide, and the fourth polypeptide at about day 1, day 2, day 3, day 4, day 5, day 10, day 20, day 30, day 40, day 50, and/or day 60 after an initial contacting (e.g., after an initial contacting step).

In some embodiments, the methods described herein are performed for about 30 days, and the NK cell is re-contacted with the first polypeptide, the second polypeptide, the third polypeptide, and the fourth polypeptide at about day 10 and at about day 20 after an initial contacting (e.g., after an initial contacting step).

In some embodiments, one or more of the first polypeptide, the second polypeptide, the third polypeptide, and the fourth polypeptide are present in a liquid culture medium. In some embodiments, two or more of the first polypeptide, the second polypeptide, the third polypeptide, and the fourth polypeptide are present in a liquid culture medium. In some embodiments, three or more of the first polypeptide, the second polypeptide, the third polypeptide, and the fourth polypeptide are present in a liquid culture medium. In some embodiments, the first polypeptide, the second polypeptide, the third polypeptide, and the fourth polypeptide are present in a liquid culture medium.

In some embodiments, the liquid culture medium includes a chemically-defined liquid culture medium (e.g., any of the liquid culture mediums described herein). For example, suitable liquid culture mediums include AIM-V medium, NK MACS medium, EL837 medium, X-VIVO 10 medium, X-VIVO 15 medium, X-VIVO 20 medium, LGM-3 lymphocyte growth medium, and LYMPHOONE T-cell expansion xeno-free medium. In some embodiments, the liquid culture medium includes additional reagents. For example, one or more additional reagents can further be included in the liquid culture medium, e.g., serum, a serum replacement (e.g., CTS Immune Cell SR serum replacement, KNOCKOUT serum replacement, Panexin NTS Pharma Grade), penicillin, streptomycin, HEPES, and/or L-alanyl-L-glutamine.

In some embodiments, the liquid culture medium includes IL-2 or a functional fragment thereof (e.g., any of the IL-2 proteins and functional fragments thereof described herein at any of the concentrations described herein). In some embodiments, the liquid culture medium includes IL-18 or a functional fragment thereof (e.g., any of the IL-18 proteins and functional fragments thereof described herein at any of the concentrations described herein). In some embodiments, the liquid culture medium includes both IL-2 or a functional fragment thereof (e.g., any of the IL-2 proteins and functional fragments thereof described herein at any of the concentrations described herein) and IL-18 or a functional fragment thereof (e.g., any of the IL-18 proteins and functional fragments thereof described herein at any of the concentrations described herein).

In some embodiments, the liquid culture medium includes an anti-CD16 antibody or antigen-binding fragment thereof. In some embodiments, the liquid culture medium does not include IL-12 or a functional fragment thereof.

In some embodiments, the method includes replenishing the liquid culture medium one or more times. For example, the liquid culture medium is replenished 2, 3, 4, 5, 6, 7, 8, 9, 10, 12, 14, 16, 18, 20, 22, 24, 26, 28, 30 or more times. In some embodiments, replenishing the liquid culture medium is performed using batch fed culturing. In some embodiments, replenishing the liquid culture medium is performed using perfusion culturing. In some embodiments, replenishing is performed by replacement of the liquid culture medium. In some embodiments, replenishing the liquid culture medium occurs about every day, about every two days, about every 3 days, about every four days, about every five days, about every six days, about every seven days, about every eight days, about every nine days, about every ten days, about every eleven days, or about every twelve days. In some embodiments, replenishing the liquid culture medium occurs about every one or two days.

In some embodiments, one or more of the first polypeptide, the second polypeptide, the third polypeptide, and the fourth polypeptide are directly or indirectly attached to one or more substrate(s) (e.g., any of the substrates described herein). In some embodiments, two or more of the first polypeptide, the second polypeptide, the third polypeptide, and the fourth polypeptide are directly or indirectly attached to one or more substrate(s). In some embodiments, three or more of the first polypeptide, the second polypeptide, the third polypeptide, and the fourth polypeptide are directly or indirectly attached to one or more substrates. In some embodiments, the first polypeptide, the second polypeptide, the third polypeptide, and the fourth polypeptide are directly or indirectly attached to one or more substrate(s).

In some embodiments, one or more of the first polypeptide, the second polypeptide, the third polypeptide, and the fourth polypeptide are directly attached to the one or more substrate(s). In some embodiments, two or more of the first polypeptide, the second polypeptide, the third polypeptide, and the fourth polypeptide are directly attached to the one or more substrate(s). In some embodiments, three or more of the first polypeptide, the second polypeptide, the third polypeptide, and the fourth polypeptide are directly attached to the one or more substrates. In some embodiments, the first polypeptide, the second polypeptide, the third polypeptide, and the fourth polypeptide are directly attached to the one or more substrate(s).

In some embodiments, one or more of the first polypeptide, the second polypeptide, the third polypeptide, and the fourth polypeptide are indirectly attached to the one or more substrate(s). In some embodiments, two or more of the first polypeptide, the second polypeptide, the third polypeptide, and the fourth polypeptide are indirectly attached to one or more substrate(s). In some embodiments, three or more of the first polypeptide, the second polypeptide, the third polypeptide, and the fourth polypeptide are indirectly attached to one or more substrate(s). In some embodiments, the first polypeptide, the second polypeptide, the third polypeptide, and the fourth polypeptide are indirectly attached to the one or more substrate(s).

In some embodiments, one or more of the first polypeptide, the second polypeptide, the third polypeptide, and the fourth polypeptide is attached (e.g., directly or indirectly) to the same substrate (e.g., bead or flask). In some embodiments, one or more of the first polypeptide, the second polypeptide, the third polypeptide, and the fourth polypeptide is attached to two or more separate substrates (e.g., bead or flask). In some embodiments, the first polypeptide is attached to a first substrate, the second polypeptide is attached to a second substrate, the third polypeptide is attached to a third substrate, and the fourth polypeptide is attached to a fourth substrate. In some embodiments, the first polypeptide and the second polypeptide are attached to a first substrate, and the third polypeptide and the fourth polypeptide are attached to a second substrate. In some embodiments, the first and third polypeptide are attached to a first substrate, and the second polypeptide and the fourth polypeptide are attached to a second substrate. In some embodiments, the first polypeptide and the fourth polypeptide are attached to a first substrate, and the second polypeptide and the third polypeptide are attached to a second substrate. In some embodiments, one or more of the first polypeptide, the second polypeptide, the third polypeptide and the fourth polypeptide is attached to a substrate (e.g., the same or different substrates), and one or more of the first polypeptide, the second polypeptide, the third polypeptide, and the fourth polypeptide is in solution (e.g., in a cell culture medium). In some embodiments, one or more of the first polypeptide, the second polypeptide, the third polypeptide, and the fourth polypeptide includes a tag. In some embodiments, two or more of the first polypeptide, the second polypeptide, the third polypeptide, and the fourth polypeptide include a tag. In some embodiments, three or more of the first polypeptide, the second polypeptide, the third polypeptide, and the fourth polypeptide include a tag. In some embodiments, the first polypeptide, the second polypeptide, the third polypeptide, and the fourth polypeptide include a tag. In some embodiments, the one or more substrate(s) includes an agent that specifically binds to the tag. In some embodiments, the tag is an Fc domain, a myc tag, a hemagglutinin (HA) tag, or a glutathione S-transferase GST tag. In some embodiments, the tag is an Fc domain, and the agent that specifically binds to the tag is an anti-Fc antibody or antigen-binding fragment thereof. In some embodiments, the tag is a myc tag and the agent is an anti-myc tag antibody or antigen-binding fragment thereof. In some embodiments, the tag is an HA tag and the agent that specifically binds to the tag is an anti-HA tag antibody or antigen-binding fragment thereof. In some embodiments, the tag is a GST tag and the agent that specifically binds to the tag is an anti-GST tag antibody or antigen-binding fragment thereof. In some embodiments, the one or more substrate(s) includes a hydrogel. In some embodiments, the one or more substrate(s) includes a natural polymer (e.g., any of the natural polymers described herein). In some embodiments, the bead includes a synthetic polymer (e.g., any of the synthetic polymers described herein). In some embodiments, the one or more substrate(s) includes a co-polymer, where the co-polymer is a co-polymer of natural polymers (e.g., any of the natural polymers described herein). In some embodiments, the one or more substrate(s) includes a co-polymer, where the co-polymer is a co-polymer of synthetic polymers (e.g., any of the synthetic polymers described herein). In some embodiments, the one or more substrate(s) includes a co-polymer, wherein the co-polymer is a co-polymer of a natural polymer and a synthetic polymer. In some embodiments, the one or more substrate(s) is a bead (e.g., a magnetic bead).

In some embodiments, the one or more substrate(s) is a culture vessel. For example, the culture vessel can be a cell culture flask, cell culture bag, tube, plate, dish, or well (e.g., round bottomed or flat bottomed well). In some embodiments, more than one and/or different culture vessels are used.

In some embodiments, contacting is performed in the presence of a liquid culture medium (e.g., any of the liquid culture mediums described herein).

In some embodiments, the first polypeptide, the second polypeptide, the third polypeptide, and the fourth polypeptide are present (e.g., in a liquid culture medium or attached to one or more substrate(s) described herein) in approximately equal amounts or concentrations.

In some embodiments, the method further includes, before the contacting step, culturing the NK cell in the presence of a feeder cell (e.g., a feeder cell provided herein). In some embodiments, the method further includes, after the contacting step, culturing the NK cell in the presence of a feeder cell.

In some embodiments, the method further includes after the contacting step, culturing the NK cell in a liquid culture medium not including each of (i) a first polypeptide comprising B7-H6 or a functional fragment thereof, or an anti-NKp30 antigen-binding domain, (ii) a second polypeptide comprising 4-1BBL or a functional fragment thereof, (iii) a third polypeptide comprising ICAM-1 or a functional fragment thereof, and (iv) a fourth polypeptide including IL-21 or a functional fragment thereof. In some embodiments, the method further includes after the contacting step, culturing the NK cell in a liquid culture medium not including a first polypeptide comprising B7-H6 or a functional fragment thereof, or an anti-NKp30 antigen-binding domain. In some embodiments, the method further includes after the contacting step, culturing the NK cell in a liquid culture medium not including a second polypeptide comprising 4-1BBL or a functional fragment thereof. In some embodiments, the method further includes after the contacting step, culturing the NK cell in a liquid culture medium not including a third polypeptide comprising ICAM-1 or a functional fragment thereof. In some embodiments, the method further includes after the contacting step, culturing the NK cell in a liquid culture medium not including a fourth polypeptide including IL-21 or a functional fragment thereof.

In some embodiments, the method further includes, before the contacting step, introducing into the NK cell a nucleic acid encoding one or more exogenous polypeptides by any suitable means (e.g., transfection, transduction, using a transposon system, and electroporation). In some embodiments, the method further includes, after the contacting step, introducing into the NK cell a nucleic acid encoding one or more exogenous polypeptides. In some embodiments, the one or more exogenous polypeptides is a CAR. In some embodiments, the one or more exogenous polypeptides is a therapeutic protein. In some embodiments, the NK cell is a genetically engineered NK cell.

In some embodiments, the method results in expansion of NK cells (e.g., any of the fold-expansion yields described herein).

### Cells

Also provided herein are NK cells (e.g., any of the exemplary NK cells described herein or known in the art) produced using any of the methods described herein.

In some embodiments, the NK cell includes a nucleic acid encoding one or more exogenous polypeptides. In some embodiments, the one or more exogenous polypeptides is a chimeric antigen receptor. In some embodiments, the one or more exogenous polypeptides is a therapeutic protein. In some embodiments, the NK cell includes one or more exogenous polypeptides. In some embodiments, the one or more exogenous polypeptides is a chimeric antigen receptor. In some embodiments, the one or more exogenous polypeptides is a therapeutic protein.

In some embodiments, the NK cells produced using any of the methods described herein are genetically engineered NK cells. In some embodiments, the NK cells is genetically engineered to include one or more exogenous nucleic acids. In some embodiments, the NK cell is genetically engineered to edit (e.g., knock-out) a nucleic acid (e.g., a gene or promoter). In some embodiments, the NK cell is genetically engineered, e.g., using methods known in the art, during the methods provided herein. In some embodiments, the NK cell has been genetically engineered prior to being used in any of the methods provided herein. In some embodiments, the NK cell is genetically engineered after being subjected to any of the methods provided herein.

In some embodiments, the NK cell is a CD56⁺CD3⁻ NK cell, a CD56⁺CD16⁺CD3⁻ NK cell, or a CD56⁺CD16⁻CD3⁻NK cell. The NK cells described herein may be from any source including tissues (e.g., lymph nodes, spleen, liver, lungs, and intestines) or has been differentiated from an induced pluripotent stem cell (iPS) or embryonic stem cell (ESC). In some embodiments, the NK cell is present in or derived from a heterogeneous cell population. In some embodiments, the NK cell is from a biological sample (e.g., a human biological sample). In some embodiments, the biological sample is or contains blood, plasma, serum, cerebrospinal fluid, synovial fluid, peripheral blood mononuclear cells (PBMCs), mobilized peripheral blood, bone marrow, cord blood, white blood cells, an apheresis product, or a leukapheresis product. In some embodiments, the NK cell is an isolated NK cell (e.g., isolated from a biological sample provided herein). In some embodiments, the NK cell has been obtained from a biological sample subjected to one or more processing steps (e.g., separation, centrifugation, washing and/or incubation). Methods of isolating NK cells from a biological sample are known in the art (see, e.g., Pak-Wittel et al. (2014) Curr. Protoc. Immunol. 105: 3.22.1-3.22.9; human NK Cell Isolation Kit (Miltenyi Biotec; Cat. No. 130-092-657). In some embodiments, the NK cell is derived from a cell line, e.g., an NK cell line including, e.g., the cell lines NK-92, NKG, YT, NK-YS, HANK-1, YTS, and NKL.

In some embodiments, the NK cells produced using any of the methods described herein are gene edited. As used herein “gene editing,” “gene edited,” and “editing” refers to a type of genetic engineering in which DNA is inserted, replaced, or removed from a genome using any gene editing tool known in the art. Non-limiting examples of gene editing tools include, without limitation, nucleases such as clustered regularly interspaced short palindromic repeats (CRISPR) associated nuclease, a transcription activator like effector nuclease (TALEN), a zinc finger nuclease, and transposases. In some embodiments, the NK cells are gene edited (e.g., using any of the exemplary methods described herein) after expansion using the methods described herein. In some embodiments, the NK cells are gene edited (e.g., using any of the exemplary methods described herein) before the NK cells are expanded using the methods described herein.

As used herein the terms “target site,” “target sequence,” “target site sequence,” “target DNA,” and “target locus” are used interchangeably and refer to a polynucleotide sequence such as, but not limited to, a nucleotide sequence on a chromosome or any other DNA molecule in the genome of an NK cell, at which a guide polynucleotide/Cas endonuclease or other gene editing tools described herein complex can recognize, bind to, and optionally nick or cleave. The target site can be an endogenous site in the genome of an NK cell, or alternatively, the target site can be heterologous to the cell and thereby not be naturally occurring in the genome of the NK cell, or the target site can be found in a heterologous genomic location compared to where it occurs in nature.

In some embodiments, the knocking out or knocking down a target sequence is performed using a gene editing system such as a CRISPR/Cas system (see e.g., Sanders and Joung, Nature Biotechnol. 32:347-55, (2014); Huang et al., J. Cell Physiol. 10:1-17, (2017) and Mitsunobu et al., Trends Biotechnol. 17:30132-4, (2017)). Generally, CRISPR/Cas systems include a CRISPR-associated protein (“Cas”) protein and at least one ribonucleic acid that is capable of directing the Cas protein to and hybridizing to a target sequence in the NK cell (e.g., expanded NK cells and/or non-expanded NK cells). The Cas protein can cleave the target sequence which results in a double-stranded break or a single-stranded break. Any CRISPR/Cas system that is capable of altering a target polynucleotide sequence in an NK cell can be used in methods described herein. In some embodiments, the CRISPR/Cas system is a CRISPR type I system. In some embodiments, the CRISPR/Cas system is a CRISPR type II system. In some embodiments, the CRISPR/Cas system is a CRISPR type V system.

Cas proteins used in the methods described herein can be a naturally occurring Cas protein or a functional derivative thereof. A “functional derivative” includes, but is not limited to, fragments of a native sequence and derivatives of a native sequence polypeptide and its fragments, provided that they have a biological activity in common with the corresponding native sequence polypeptide. Biological activity contemplated herein is the ability of the functional derivative to hydrolyze a DNA substrate (e.g., a target sequence) into fragments. Additionally, the term “derivative” encompasses both amino acid sequence substitutions of a polypeptide, covalent modifications, and fusions thereof. Suitable derivatives of a Cas protein or a fragment thereof include but are not limited to mutants, fusions, or covalent modifications of Cas protein.

In some embodiments, the Cas protein used in the methods described herein include Cas9 protein, Cpf1 protein, C2c1 protein, C2c2 protein, C2c3 protein, Cas3 protein, Cas5 protein, Cas7 protein, Cas8 protein, Cas10 protein, functional derivatives thereof, and complexes thereof. The double-stranded endonuclease activity of Cas9 protein typically requires a short conserved sequence (e.g., 2-5 nucleotides), known as a protospacer-associated motif (PAM), which follows immediately after the 3′ end of a target sequence.

The terms “single guide RNA,” “sgRNA,” “guide RNA,” or “gRNA” are used interchangeably herein and relate to a fusion of two RNA molecules, a crRNA (CRISPR RNA) including a variable targeting domain fused to a tracrRNA (trans-activating CRISPR RNA). The single guide RNA can comprise a crRNA or crRNA fragment and a tracrRNA or tracrRNA fragment that can form a complex with a Cas endonuclease, where the guide RNA/Cas endonuclease complex can direct the Cas endonuclease to a DNA target site, enabling the Cas endonuclease to recognize, bind to, and optionally nick or cleave (introduce a single or double strand break) the DNA target site. The single guide polynucleotide being comprised of sequences from the crRNA and the tracrRNA may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). In some embodiments, the one or two ribonucleic acids are designed to hybridize to a target sequence immediately adjacent to a deoxyribonucleic acid sequence recognized by the Cas protein. Guide RNAs can also be designed using available software. In some embodiments, the one or more sgRNAs can be transfected into NK cells, according to methods known in the art.

In some embodiments, the target sequence to which the Cas protein is directed by a guide RNA (or components thereof), can be between about 15 and about 25 nucleotides in length (e.g., 15, 16, 17, 18, 19, 20, 21, 22, 23, 24, or 25 nucleotides in length). In some embodiments, the target sequence is at least 20 nucleotides in length. In some embodiments, the target sequence immediately precedes a short conserved sequence known as PAM, recognized by the Cas protein. In some embodiments, the target sequences can be selected to minimize off-target effects of the CRISPR/Cas systems. Those skilled in the art will appreciate that a variety of techniques can be used to select suitable target sequences for minimizing off-target effects (e.g., such as the use of bioinformatics analyses).

In some embodiments, the Cas protein is introduced into NK cells in polypeptide form. In some embodiments, an NK cell can be genetically engineered to produce the Cas protein (e.g., by introducing mRNA encoding the Cas protein into the NK cell).

Gene editing tools include TAL effector nucleases (TALENs) which are a class of sequence-specific nucleases that can be used to make double-strand breaks at specific target sequences in the genome of a plant or other organism (see, e.g., Miller et al. Nature Biotechnol. 29:143-148 (2011)). Endonucleases are enzymes that cleave the phosphodiester bond within a polynucleotide chain. Endonucleases include restriction endonucleases, which cleave DNA at specific sites without damaging the bases, and meganucleases, also known as homing endonucleases (“HEases”), which similar to restriction endonucleases, bind and cut at a specific recognition site, however the recognition sites for meganucleases are typically longer, about 18 bp or more. Meganucleases have been classified into four families based on conserved sequences. These motifs participate in the coordination of metal ions and hydrolysis of phosphodiester bonds. HEases are notable for their long recognition sites, and for tolerating some sequence polymorphisms in their DNA substrates. One step in the recombination process involves polynucleotide cleavage at or near the recognition site. The cleaving activity can be used to produce a double-strand break. For reviews of site-specific recombinases and their recognition sites, see, Sauer, Curr. Op. Biotechnol. 5:521-7 (1994); and Sadowski, FASEB J. 7:760-7 (1993), each of which is incorporated herein by reference in its entirety. In some examples, the recombinase is from the integrase or resolvase families.

Zinc finger nucleases (ZFNs) are gene-editing tools that can engineer double-strand breaks in DNA of an NK cell (e.g., expanded NK cells or non-expanded NK cells). ZFNs include a zinc finger DNA binding domain and a double-strand-break-inducing agent domain. Target sequence specificity is conferred by the zinc finger domain, which typically includes two, three, or four zinc fingers. One example of a zinc finger domain structure is C2H2, however other zinc finger structures are known in the art. Zinc finger domains are useful for designing polypeptides which specifically bind a target sequence. ZFNs can include an engineered DNA-binding zinc finger domain linked to a non-specific endonuclease domain, for example nuclease domain from a Type IIs endonuclease such as FokI. Additional functional domain can be fused to the zinc-finger binding domain, including transcriptional activator domains, transcription repressor domains, and methylases. In some examples, dimerization of the nuclease domain is required for cleavage activity. Generally, each zinc finger recognizes three consecutive base pairs in the target sequence, such that, a 3 finger domain recognizes a sequence of 9 contiguous nucleotides. In cases where dimerization is required, two sets of zinc finger triplets are used to bind an 18 nucleotide target sequence, for example.

Gene editing tools, such as transposons and systems thereof, can be used to edit feeder-free expanded NK cells or non-expanded NK cells. Transposon systems can include a transcriptional unit of a nucleic acid construct, i.e., the regulatory region operably linked to a target nucleic acid sequence, flanked by an inverted repeat of a transposon. Examples of transposon systems, include Sleeping Beauty (see, U.S. Pat. No. 6,613,752 and U.S. Publication No. 2005/0003542); Frog Prince (Miskey et al., Nucleic Acids Res., 31:6873 (2003)); Tol2 (Kawakami, Genome Biology, 8 (Suppl. 1):57 (2007); Minos (Pavlopoulos et al., Genome Biol. 8 (Suppl. 1): S2 (2007)); Hsmar 1 (Miskey et al., Mol. Cell Biol., 27:4589 (2007)); and Passport have been developed to introduce nucleic acids into cells.

In some embodiments, a polynucleotide is introduced into a NK cell using a transposon system. In some embodiments, the transposon system comprises a vector comprising transposable elements and a polynucleotide encoding an exogenous protein contemplated herein; and a transposase. In some embodiments, the transposon vector system is a single transposase vector system, see, e.g., International Publ. No. WO 2008/027384. Exemplary transposases include, but are not limited to: TcBuster, piggyBac, Sleeping Beauty, Mosl, Tcl/mariner, Tn5, Tn7, Tol2, mini-Tol2, Tc3, MuA, Himar I, Frog Prince, and derivatives thereof. The piggyback transposon and transposase are described, for example, in U.S. Pat. No. 6,962,810, which is incorporated herein by reference in its entirety. The Sleeping Beauty transposon and transposase are described, for example, in Izsvak et al., J. Mol. Biol. 302: 93-102 (2000), which is incorporated herein by reference in its entirety. The Tol2 transposon which was first isolated from the medaka fish Oryzias latipes and belongs to the hAT family of transposons is described in Kawakami et al. (2000). Mini-Tol2 is a variant of Tol2 and is described in Balciunas et al. (2006). The Tol2 and Mini-Tol2 transposons facilitate integration of a transgene into the genome of an organism when co-acting with the Tol2 transposase. The Frog Prince transposon and transposase are described, for example, in Miskey et al., Nucleic Acids Res. 31:6873-6881 (2003).

A transposase can be delivered as a protein, encoded on the same nucleic acid construct as the target nucleic acid, can be introduced on a separate nucleic acid construct, or provided as an mRNA (e.g., an in vitro-transcribed and capped mRNA).

Gene editing tools as described herein can be introduced into an NK cell by any method known in the art, such as, but not limited to, topical application, transient introduction methods, transfection, delivery via cell-penetrating peptides, electroporation, whiskers mediated delivery, microinjection, particle mediated delivery, or mesoporous silica nanoparticle (MSN)-mediated direct delivery. In some embodiments, gene editing tools as described herein are introduced into NK cells (e.g., expanded NK cells or NK cells) via a vector. Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., HIV-1, SIV or FIV), retrovirus (e.g., ASV, ALV or MoMLV), and transposons (e.g., TcBuster, Sleeping Beauty, P-elements, Tol-2, Frog Prince, piggyBac).

### Pharmaceutical Compositions

Also provided herein are compositions (e.g., pharmaceutical compositions) that include an NK cell produced using any of the methods described herein. In some embodiments, the compositions (e.g., pharmaceutical compositions) are formulated for different routes of administration (e.g., intravenous or intratumoral administration). In some embodiments, the compositions (e.g., pharmaceutical compositions) can include a pharmaceutically acceptable carrier (e.g., phosphate buffered saline). Single or multiple administrations of any of the pharmaceutical compositions described herein can be administered to a subject depending on, for example: the dosage and frequency as required and tolerated by the patient. A dosage of the pharmaceutical composition should provide a sufficient quantity of the NK cell (e.g., any of the NK cells described herein) to effectively treat or ameliorate conditions, diseases, or symptoms in a subject in need thereof.

### Methods of Treatment

Also provided herein are methods of treating a subject in need thereof including administering to the subject an NK cell (e.g., any of the NK cells produced using any of the methods described herein). Also provided herein are methods of treating a subject in need thereof including administering to the subject a pharmaceutical composition (e.g., any of the pharmaceutical compositions described herein).

Also provided herein are methods of treating a subject having a cancer or an infection that include administering a therapeutically effective amount of any of the compositions or pharmaceutical compositions provided herein.

### Kits

Provided herein are kits including one or more (e.g., two or three) substrate(s) that together include: (i) a first polypeptide comprising B7-H6 or a functional fragment thereof, or an anti-NKp30 antigen-binding domain, (ii) a second polypeptide comprising 4-1BBL or a functional fragment thereof, and (iii) a third polypeptide including ICAM-1 or a functional fragment thereof, directly or indirectly attached to the one or more substrate(s).

The first polypeptide can be any of the exemplary first polypeptides described herein. The second polypeptide can be any of the exemplary second polypeptides described herein. The third polypeptide can be any of the exemplary third polypeptides described herein.

In some embodiments, the first polypeptide, the second polypeptide, and the third polypeptide are directly attached to the one or more substrate(s) (e.g., any of the substrates provided herein). In some embodiments, the first polypeptide, the second polypeptide, and the third polypeptide are indirectly attached to the one or more substrate(s). In some embodiments, the first polypeptide, the second polypeptide, and the third polypeptide include a tag. In some embodiments, the one or more substrate(s) includes an agent that specifically binds to the tag. In some embodiments, the tag is an Fc domain, a myc tag, a HA tag, or a GST tag. In some embodiments, the tag is an Fc domain, and the agent that specifically binds to the tag is an anti-Fc antibody or antigen-binding fragment thereof. In some embodiments, the tag is a myc tag and the agent is an anti-myc tag antibody or antigen-binding fragment thereof. In some embodiments, the tag is an HA tag and the agent that specifically binds to the tag is an anti-HA tag antibody or antigen-binding fragment thereof. In some embodiments, the tag is a GST tag and the agent that specifically binds to the tag is an anti-GST tag antibody or antigen-binding fragment thereof. In some embodiments, the first polypeptide, the second polypeptide, and the third polypeptide are present in or on the one or more substrate(s) in approximately equal amounts or concentrations.

In some embodiments, the one or more substrate(s) is a bead. In some embodiments, the substrate includes a hydrogel. In some embodiments, the substrate includes a natural polymer, a synthetic polymer, or a co-polymer. For example, the natural polymer can be any of the exemplary natural polymers described herein. In some embodiments, the synthetic polymer can be any of the exemplary synthetic polymers described herein.

In some embodiment, the substrate includes a co-polymer, where the co-polymer is a copolymer of natural polymers (e.g., any of the exemplary natural polymers described herein). In some embodiments, the substrate includes a co-polymer of synthetic polymers (e.g., any of the exemplary synthetic polymers described herein). In some embodiments, the substrate includes a copolymer of a natural polymer (e.g., any of the exemplary natural polymers described herein) with a synthetic polymer (e.g., any of the exemplary synthetic polymers described herein).

In some embodiments, the substrate is a bead. In some embodiments, the bead is a magnetic bead.

In some embodiments, the one or more substrate(s) is a culture vessel. In some embodiments, the culture vessel is a cell culture flask, cell culture bag, tube, plate, dish, or well.

In some embodiments, the kit includes a liquid culture medium. In some embodiments, the liquid culture medium is a chemically-defined liquid culture medium. In some embodiments, the liquid culture medium is AIM-V medium, NK MACS medium, EL837 medium, X-VIVO 10 medium, X-VIVO 15 medium, X-VIVO 20 medium, LGM-3 lymphocyte growth medium, or LYMPHOONE T-cell expansion xeno-free medium.

In some embodiments, the liquid culture medium includes one or more of: serum, a serum replacement (e.g., CTS Immune Cell SR serum replacement, KNOCKOUT serum replacement, Panexin NTS Pharma Grade), penicillin, streptomycin, HEPES, and/or L-alanyl-L-glutamine.

In some embodiments, the liquid culture medium includes IL-2 or a functional fragment thereof (e.g., any of the exemplary IL-2 proteins and functional fragments thereof described herein at any of the exemplary concentrations described herein). In some embodiments, the liquid culture medium includes IL-18 or a functional fragment thereof (e.g., any of the exemplary IL-18 proteins and functional fragments thereof described herein at any of the exemplary concentrations described herein). In some embodiments, the liquid culture medium includes IL-2 or a functional fragment thereof (e.g., any of the exemplary IL-2 proteins and functional fragments thereof described herein at any of the exemplary concentrations described herein) and IL-18 or a functional fragment thereof (e.g., any of the exemplary IL-18 proteins and functional fragments thereof described herein at any of the exemplary concentrations described herein).

In some embodiments, the liquid culture medium includes an anti-CD16 antibody or antigen-binding fragment thereof.

In some embodiments, the liquid culture medium does not include IL-12 or a functional fragment thereof. In some embodiments, the liquid culture medium includes IL-21 or a functional fragment thereof (e.g., any of the exemplary IL-21 proteins and functional fragments thereof described herein at any of the exemplary concentrations described herein). In some embodiments, the liquid culture medium does not include IL-21 or a functional fragment thereof.

Also provided herein are kits including: one or more substrate(s) that together include: (i) a first polypeptide comprising B7-H6 or a functional fragment thereof, or an anti-NKp30 antigen-binding domain, (ii) a second polypeptide comprising 4-1BBL or a functional fragment thereof, (iii) a third polypeptide comprising ICAM-1 or a functional fragment thereof, and (iv) a fourth polypeptide comprising IL-21 or a functional fragment thereof, directly or indirectly attached to the one or more substrate(s).

The first polypeptide can be any of the exemplary first polypeptides described herein. The second polypeptide can be any of the exemplary second polypeptides described herein. The third polypeptide can be any of the exemplary third polypeptides described herein. The fourth polypeptide can be any of the exemplary fourth polypeptides described herein.

In some embodiments, the first polypeptide, the second polypeptide, the third polypeptide, and the fourth polypeptide are directly attached to the one or more substrate(s) (e.g., any of the substrates described herein). In some embodiments, the first polypeptide, the second polypeptide, the third polypeptide, and the fourth polypeptide are indirectly attached to the one or more substrate(s). In some embodiments, the first polypeptide, the second polypeptide, the third polypeptide, and the fourth polypeptide include a tag. In some embodiments, the one or more substrate(s) include(s) an agent that specifically binds to the tag. In some embodiments, the tag is an Fc domain, a myc tag, a HA tag, or a GST tag. In some embodiments, the tag is an Fc domain, and the agent that specifically binds to the tag is an anti-Fc antibody or antigen-binding fragment thereof. In some embodiments, the tag is a myc tag and the agent is an anti-myc tag antibody or antigen-binding fragment thereof. In some embodiments, the tag is an HA tag and the agent that specifically binds to the tag is an anti-HA tag antibody or antigen-binding fragment thereof. In some embodiments, the tag is a GST tag and the agent that specifically binds to the tag is an anti-GST tag antibody or antigen-binding fragment thereof.

In some embodiments, the first polypeptide, the second polypeptide, the third polypeptide, and the fourth polypeptide are present in or on the one or more substrate(s) at approximately equal amounts or concentrations.

In some embodiments, the substrate includes a hydrogel. In some embodiments, the substrate includes a natural polymer, a synthetic polymer, or a co-polymer. In some embodiments, the natural polymer can be any of the exemplary natural polymers described herein. In some embodiments, the synthetic polymer can be any of the exemplary synthetic polymers described herein. In some embodiments, the co-polymer is a copolymer of natural polymers, a copolymer of synthetic polymers or a copolymer of a natural polymer with a synthetic polymer.

In some embodiments, the one or more substrate(s) is a bead. In some embodiments, the bead is a magnetic bead.

In some embodiments, the one or more substrate(s) is a culture vessel. In some embodiments, the culture vessel is a cell culture flask, cell culture bag, tube, plate, dish, or well.

In some embodiments, the kit includes a liquid culture medium. In some embodiments, the liquid culture medium is a chemically-defined liquid culture medium. In some embodiments, the liquid culture medium is selected from the group consisting of: AIM-V medium, NK MACS medium, EL837 medium, X-VIVO 10 medium, X-VIVO 15 medium, X-VIVO 20 medium, LGM-3 lymphocyte growth medium, and LymphoONE T-cell expansion xeno-free medium.

In some embodiments, the liquid culture medium includes one or more of: serum, a serum replacement (e.g., CTS Immune Cell SR serum replacement, KNOCKOUT serum replacement, Panexin NTS Pharma Grade), penicillin, streptomycin, HEPES, and/or L-alanyl-L-glutamine.

In some embodiments, the liquid culture medium includes IL-2 or a functional fragment thereof (e.g., any of the exemplary IL-2 proteins or functional fragments thereof described herein at any of the exemplary concentrations described herein). In some embodiments, the liquid culture medium includes IL-18 or a functional fragment thereof (e.g., any of the exemplary IL-18 proteins and functional fragments thereof described herein at any of the exemplary concentrations described herein). In some embodiments, the liquid culture medium includes IL-2 or a functional fragment thereof (e.g., any of the exemplary IL-2 proteins and functional fragments thereof described herein at any of the exemplary concentrations described herein) and IL-18, or a functional fragment thereof (e.g., any of the exemplary IL-18 proteins and functional fragments thereof described herein at any of the exemplary concentrations described herein).

In some embodiments, the liquid culture medium includes an anti-CD16 antibody or antigen-binding fragment thereof. In some embodiments, the liquid culture medium does not include IL-12 or a functional fragment thereof.

### EXAMPLES

### Example 1. Feeder-Free NK Cell Expansion Methods Result in Robust NK Cell Expansion

To test the expansion yield of a new feeder-free NK cell expansion method, the following experiments were performed using peripheral blood NK cells from 2 separate donors. Briefly, non-treated round bottom 96-well cell culture plates were coated with PBS including 10 μg/mL of rabbit anti-human Fc domain antibody (“coating solution”; 200 μL/well) overnight at room temperature. After removal of the coating solution, the plates were treated with 200 μL/well of phosphate buffered saline (PBS) including (a) 1.87 nM of a first polypeptide comprising human B7-H6 and a human Fc domain tag, (b) 1.14 nM of a second polypeptide comprising human 4-1BBL and a human Fc domain tag, (c) 2.38 nM of a third polypeptide comprising human ICAM-1 and a human Fc domain tag, and (d) 1.29 nM of a fourth polypeptide comprising human IL-21 and an Fc domain tag, and incubated at room temperature for about 5 hours to conjugate the polypeptides to the flask culture surface. For a first round of expansion, at day 0, 1×10⁶ NK cells in 100 μL of AIM-V medium supplemented with 5% CTS™ immune cell serum replacement (Thermo Fisher Scientific), 10 mM HEPES, 2 mM _{L}-alanyl-_{L}-glutamine, 100 IU/mL human recombinant IL-2, 0.56 nM human recombinant IL-18, and penicillin/streptomycin, were seeded in 10 wells (i.e., 1×10⁵ NK cells/well). At day 2, one volume of culture medium containing 2× concentration of IL-2 and IL-18 (hereinafter “2×IL-2/IL-18 culture medium”) (i.e., AIM-V medium supplemented with 5% CTS™ immune cell serum replacement, 10 mM HEPES, 2 mM _{L}-alanyl-_{L}-glutamine, 200 IU/mL human recombinant IL-2, 1.12 nM human recombinant IL-18, and penicillin/streptomycin) was added to each well. At day 4, the NK cells were collected and pooled from the ten wells and transferred to an uncoated T25 cell culture flask along with one volume of 2×IL-2/IL-18 culture medium. At day 6, the NK cells were transferred to an uncoated T75 cell culture flask. At days 6, 8 and 9 (as needed), one volume of 2×IL-2/IL-18 culture medium was added to the flasks. As needed, the NK cells were split into separate uncoated T75 cell culture flasks at day 8. At day 10, the NK cells were collected, and NK cell numbers assessed using an automated cell counter. For a second round of expansion, 1×10⁶ NK cells/flask (5.0×10⁵ NK cells/mL) from the first round of expansion (i.e., NK cells from day 10) were seeded into multiple T25 T-flasks previously coated with rabbit anti-human Fc domain antibody and subsequently treated with PBS containing the concentrations of the first polypeptide comprising human B7-H6 and a human Fc domain tag, the second polypeptide comprising 4-1BBL and a human Fc domain tag, the third polypeptide comprising ICAM-1 and a human Fc domain tag, and the fourth polypeptide comprising IL-21 and an Fc domain tag (as described above) to conjugate the polypeptides to the flask culture surface. At day 12, one volume of 2×IL-2/IL-18 culture medium was added to each flask. At day 14, the NK cells were transferred to an uncoated T75 cell culture flask along with one volume of 2×IL-2/IL-18 culture medium. At days 16, 18 and 19 (as needed), one volume of 2×IL-2/IL-18 culture medium was added to the flasks. At day 20, the NK cells were collected, and NK cell numbers assessed as using an automated cell counter. For a third round of expansion, the methods described above for the second round of expansion were repeated, and at day 30 the NK cell numbers were assessed as described above. Throughout the second and third rounds of expansion, as needed, the NK cells were split into separate uncoated T75 flasks.

For comparison, NK cells were also expanded using irradiated (100 Gy) membrane-bound IL21- and 4-1BBL-expressing K562 feeder cells. Briefly, for a first round of expansion, at day 0, 1×10⁶ peripheral blood NK cells (2.5×10⁵ cells/mL) from the same 2 donors used above were co-cultured with the irradiated feeder cells at a 1:2 ratio (NK cell:feeder cell) in AIM-V medium supplemented with 5% CTS™ immune cell serum replacement, 10 mM HEPES, 2 mM _{L}-alanyl-_{L}-glutamine, 100 IU/mL human recombinant IL-2, and penicillin/streptomycin, in an uncoated T25 cell culture flask. At day 2, one volume of culture medium containing 2× concentration of IL-2 (hereinafter “2×IL-2 culture medium”) (i.e., AIM-V medium supplemented with 5% CTS™ immune cell serum replacement, 10 mM HEPES, 2 mM _{L}-alanyl-_{L}-glutamine, 200 IU/mL human recombinant IL-2, and penicillin/streptomycin) was added to each cell culture flask. At day 4, the NK cells were collected and transferred to an uncoated T75 cell culture flask. At days 6, 8 and 9 (as needed), one volume of 2×IL-2 culture medium was added to the cell culture flasks. At day 10, the NK cells were collected, and NK cell numbers were assessed using an automated cell counter. For a second and third rounds of expansion, at days 10 and 20, respectively, 1×10⁶ NK cells/flask (1.25×10⁵ NK cells/mL) from the previous round of expansion (i.e., NK cells from day 10 or day 20, respectively) were seeded into multiple uncoated T75 cell culture flasks. The methods described above for the first round of expansion were repeated for the second and third rounds of expansion (for a total of 30 days) and NK cell counts assessed at days 20 and 30. As needed, the NK cells were split into separate uncoated T75 flasks.

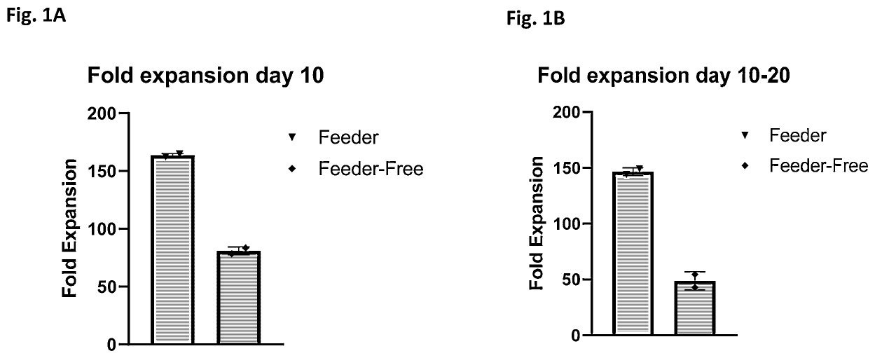

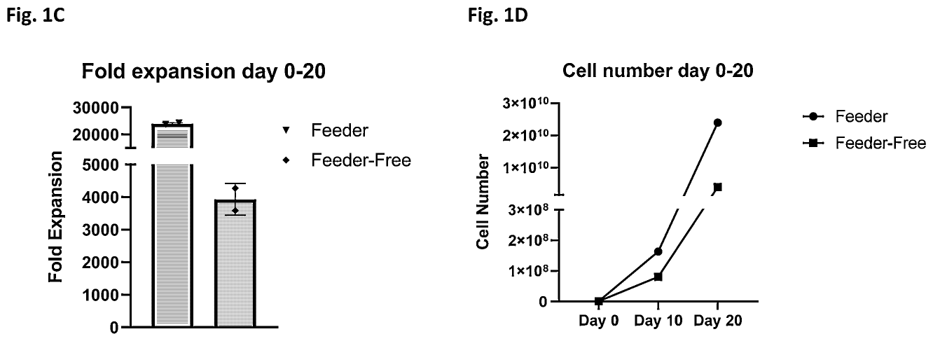

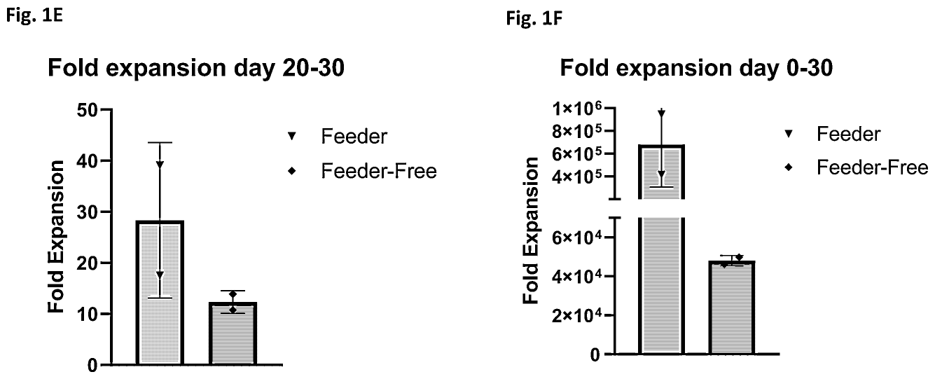

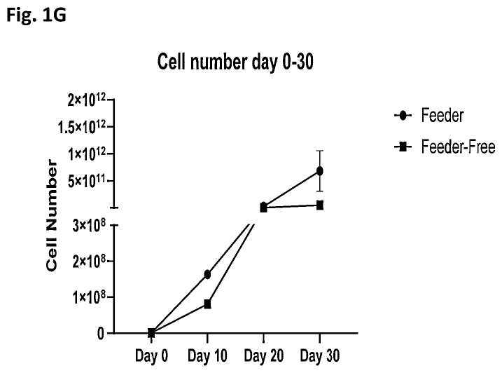

As shown in FIG. 1A, at day 10, the feeder-free expansion method resulted in an average of about 80-fold expansion of the NK cells, as compared to an average of about 160-fold expansion of the NK cells using the feeder cell expansion method. Similarly, from day 10 to day 20 (following the second round of expansion), the feeder-free expansion method resulted in an average of about 50-fold expansion of the NK cells, as compared to an average of about 150-fold expansion of the NK cells using the feeder cell expansion method (see FIG. 1B). The total expansion of NK cells through day 20 was an average of about 3,900-fold (3.9×10⁹ total NK cells starting from 1×10⁶ NK cells) using the feeder-free method as compared to an average of about 23,900-fold (2.39×10¹⁰ total NK cells starting from 1×10⁶ NK cells) using the feeder cell expansion method (see FIG. 1C and FIG. 1D). From day 20 to day 30 (following the third round of expansion), the feeder-free expansion method resulted in an average of about 12-fold expansion of the NK cells, as compared to an average of about 28-fold expansion using the feeder cell expansion method (see FIG. 1E). The total expansion of NK cells through day 30 was 47,900-fold (4.79×10¹⁰ total NK cells starting from 1×10⁶ NK cells) using the feeder-free method as compared to 681,900-fold (6.82×10¹¹ total NK cells starting from 1×10⁶ NK cells) using the feeder cell expansion method (see FIG. 1F and FIG. 1G). Overall, the above data demonstrate that the feeder-free expansion system provides for surprisingly robust and desirable in vitro expansion of NK cells.

### Example 2. Feeder-Free Expanded NK Cells Exhibit Cytolytic Activity Against Cancer Cell Lines

To examine the functional activity of NK cells expanded using the feeder-free expansion methods, the cytolytic activity of feeder-free expanded NK cells, as described in Example 1, was compared with the cytolytic activity of feeder cell-expanded NK cells using four different target cancer cell lines. Cytolytic activity was measured using a luciferase killing assay with NK cells that were expanded for 20 days (e.g., either feeder-free or feeder cell-expanded NK cells). Briefly, adherent cells (e.g., target cell lines) were trypsinized and resuspsended in 10 mL of medium. Cells were counted and added to a 96-well plate (˜1×10⁵ cells/100 μL per well). The target cancer cells expressing luciferase were incubated for 24 hours and the number of cells calculated based on their proliferation rate. Next, effector cells (e.g., feeder-free expanded NK cells or feeder cell-expanded NK cells) were counted, added at a 1:1 ratio to the wells containing the target cells, and incubated for about 24 hours. After incubation luciferin was added (280 μg/mL) to the cell mixture, incubated for 10 minutes at 37° C. in a 5% CO₂ incubator and luminescence detected on a plate reader. The above protocol was used for target cells in suspension as well.

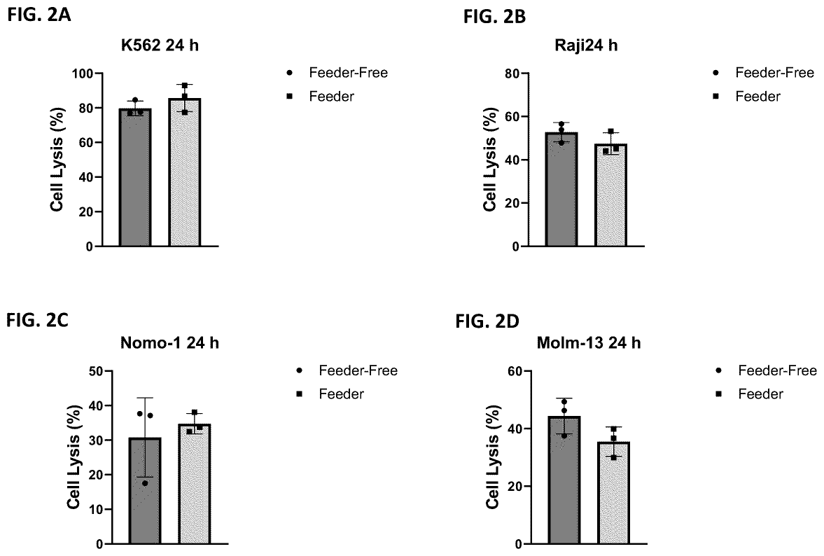

FIG. 2A is a bar graph showing the percent of cell lysis of K562 cells after 24 hours with either feeder-free expanded NK cells and feeder cell-expanded NK cells. The K562 cell line is a model for chronic myelogenous leukemia. Feeder-free expanded NK cells showed a comparable level of cell lysis compared to feeder cell-expanded NK cells. FIG. 2B is a bar graph showing the percent of cell lysis of Raji cells after 24 hours with either feeder-free expanded NK cells and feeder cell-expanded NK cells. The Raji cell line is a model for Burkitt's lymphoma. Feeder-free expanded NK cells showed improved cytolytic activity compared to feeder cell-expanded NK cells. FIG. 2C is a bar graph showing the percent of cell lysis of Nomo-1 cells after 24 hours with either feeder-free expanded NK cells and feeder cell-expanded NK cells. The Nomo-1 cell line is a model for acute myeloid leukemia. Feeder-free expanded NK cells showed a comparable level of cell lysis compared to feeder cell-expanded NK cells. FIG. 2D is a bar graph showing the percent of cell lysis of Molm-13 cells after 24 hours with either feeder-free expanded NK cells and feeder cell-expanded NK cells. The Molm-13 cell line is another model for acute myeloid leukemia. In this assay, feeder-free expanded NK cells showed improved cytolytic activity compared to feeder cell-expanded NK cells.

Collectively, the data demonstrate that feeder-free expanded NK cells have comparable cytolytic activity to feeder cell-expanded NK cells and in some cases outperform feeder cell-expanded NK cells.

### Example 3. Telomere Length of Feeder-Free Expanded NK Cells Versus Feeder-Cell Expanded NK Cells

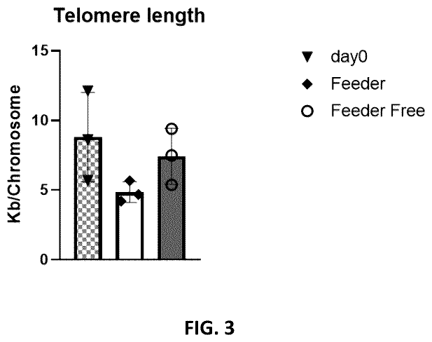

The telomere length of NK cells expanded using the feeder-free expansion methods, as described in Example 1, was compared with the telomere length of feeder cell-expanded NK cells using the Absolute Human Telomere Length Quantification (AHTLQ) qPCR Assay Kit (ScienCell™ Research Laboratories, Cat. No. 8918), as instructed by the manufacturer. The experiment was performed using NK cells from three separate donors, and telomere length was determined prior to expansion, and at day 20 following expansion using either the feeder-free expansion method or the feeder cell-expansion method. As shown in FIG. 3, NK cells expanded using the feeder-free methods exhibited similar telomere lengths as feeder cell-expanded methods.

### Example 4. Cytolytic Activity of Feeder-Free Expanded NK Cells Against Solid Tumor Cancer Cell Lines

To assess the functional activity of feeder-free expanded NK cells against solid tumors, the following experiments can be performed. Briefly, the cytolytic activity of feeder-free expanded NK cells, as described in Example 1, can be compared to the cytolytic activity of feeder cell-expanded NK cells using the methods described in Example 2, using cancer cell lines grown in adherent conditions. The following cell lines, modified to express luciferase, can be used to assess cytolytic activity against solid tumors: 786-O (a model for renal cell carcinoma), SK-OV-3 (a model for ovarian cancer), 143B U2OS (a model for osteosarcoma), and LNCap (a model for prostate cancer).

What is claimed is:

1. A method of expanding an NK cell comprising:
contacting an NK cell disposed in a liquid culture medium, in the absence of a feeder cell, with (i) a first polypeptide comprising B7-H6 or a functional fragment thereof, or an anti-NKp30 antigen-binding domain, (ii) a second polypeptide comprising 4-1BBL or a functional fragment thereof, and (iii) a third polypeptide comprising ICAM-1 or a functional fragment thereof, under conditions sufficient for expansion of the NK cell.

2. The method of claim 1, wherein after the contacting step the method comprises periodically re-contacting the NK cell with the first polypeptide, the second polypeptide, and the third polypeptide.

3. The method of claim 2, wherein the method is performed over about 30 days, and the re-contacting step is performed at about day 10 and at about day 20 after the contacting step.

4. The method of claim 1 or 2, wherein the method is performed over about 1 day to about 30 days.

5. The method of claim 4, wherein the method is performed over about 5 days to about 30 days.

6. The method of claim 5, wherein the method is performed over about 10 days to about days.

7. The method of any one of claims 1-6, wherein one or more of the first polypeptide, the second polypeptide, and the third polypeptide are present in the liquid culture medium.

8. The method of any one of claims 1-6, wherein two or more of the first polypeptide, the second polypeptide, and the third polypeptide are present in the liquid culture medium.

9. The method of any one of claims 1-6, wherein the first polypeptide, the second polypeptide, and the third polypeptide are present in the liquid culture medium.

10. The method of any one of claims 1-6, wherein one or more of the first polypeptide, the second polypeptide, and the third polypeptide are directly or indirectly attached to one or more substrate(s).

11. The method of any one of claims 1-10, wherein the first polypeptide, the second polypeptide, and the third polypeptide are present in approximately equal amounts or concentrations.

12. The method of claim 10 or 11, wherein one or more of the first polypeptide, the second polypeptide, and the third polypeptide are directly attached to the one or more substrate(s).

13. The method of claim 10 or 11, wherein one or more of the first polypeptide, the second polypeptide, and the third polypeptide are indirectly attached to the one or more substrate(s).

14. The method of any one of claims 10-13, wherein one or more of the first polypeptide, the second polypeptide, and the third polypeptide further comprises a tag.

15. The method of claim 14, wherein the one or more substrate(s) comprises an agent that specifically binds to the tag.

16. The method of claim 14 or 15, wherein the tag is an Fc domain, a myc tag, a HA tag, or a GST tag.

17. The method of claim 15 or 16, wherein: (a) the tag is an Fc domain, and the agent that specifically binds to the tag is an anti-Fc antibody or antigen-binding fragment thereof; (b) the tag is a myc tag and the agent is an anti-myc tag antibody or antigen-binding fragment thereof (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

18. The method of any one of claims 10-17, wherein one or more of the first polypeptide, the second polypeptide, and the third polypeptide further comprises a linker.

19. The method of claim 18, wherein the linker comprises a glycine-serine (GS) repeat.

20. The method of claim 18 or 19, wherein the linker comprises an IgG hinge region.

21. The method of any one of claims 1-6, wherein two or more of the first polypeptide, the second polypeptide, and the third polypeptide are directly or indirectly attached to one or more substrate(s).

22. The method of claim 22, wherein two or more of the first polypeptide, the second polypeptide, and the third polypeptide are directly attached to the one or more substrate(s).

23. The method of claim 22, wherein two or more of the first polypeptide, the second polypeptide, and the third polypeptide are indirectly attached to the one or more substrate(s).

24. The method of any one of claims 21-23, wherein two or more of the first polypeptide, the second polypeptide, and the third polypeptide further comprise a tag.

25. The method of claim 24, wherein the one or more substrate(s) comprises an agent that specifically binds to the tag.

26. The method of claim 24 or 25, wherein the tag is an Fc domain, a myc tag, a HA tag, or a GST tag.

27. The method of claim 25 or 26, wherein: (a) the tag is an Fc domain, and the agent that specifically binds to the tag is an anti-Fc antibody or antigen-binding fragment thereof (b) the tag is a myc tag and the agent is an anti-myc tag antibody or antigen-binding fragment thereof (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

28. The method of any one of claims 21-27, wherein one or more of the first polypeptide, the second polypeptide, and the third polypeptide further comprises a linker.

29. The method of claim 28, wherein the linker comprises a glycine-serine (GS) repeat.

30. The method of claim 28 or claim 29, wherein the linker comprises an IgG hinge region.

31. The method of any one of claims 1-6, wherein the first polypeptide, the second polypeptide, and the third polypeptide are directly or indirectly attached to one or more substrate(s).

32. The method of claim 31, wherein the first polypeptide, the second polypeptide, and the third polypeptide are directly attached to the one or more substrate(s).

33. The method of claim 31, wherein the first polypeptide, the second polypeptide, and the third polypeptide are indirectly attached to the one or more substrate(s).

34. The method of any one of claims 31-33, wherein the first polypeptide, the second polypeptide, and the third polypeptide further comprise a tag.

35. The method of claim 34, wherein the one or more substrate(s) comprises an agent that specifically binds to the tag.

36. The method of claim 34 or 35, wherein the tag is an Fc domain, a myc tag, a HA tag, or a GST tag.

37. The method of claim 35 or 36, wherein: (a) the tag is an Fc domain, and the agent that specifically binds to the tag is an anti-Fc antibody or antigen-binding fragment thereof; (b) the tag is a myc tag and the agent is an anti-myc tag antibody or antigen-binding fragment thereof; (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

38. The method of any one of claims 31-37, wherein one or more of the first polypeptide, the second polypeptide, and the third polypeptide further comprises a linker.

39. The method of claim 38, wherein the linker comprises a glycine-serine (GS) repeat.

40. The method of claim 38 or 39, wherein the linker comprises an IgG hinge region.

41. The method of any one of claims 10-40, wherein the substrate comprises a hydrogel.

42. The method of any one of claims 10-40, wherein the substrate comprises a natural polymer, a synthetic polymer, or a co-polymer.

43. The method of claim 42, wherein the natural polymer is selected from the group consisting of: alginate, agarose, carrageenan, chitosan, dextran, carboxymethylcellulose, heparin, hyaluronic acid, polyamino acids, collagen, gelatin, fibrin, fibrous protein-based biopolymers, and any combination thereof.

44. The method of claim 42, wherein the synthetic polymer is selected from the group consisting of: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene)), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof.

45. The method of claim 42, wherein the co-polymer is a copolymer of natural polymers, a copolymer of synthetic polymers or a copolymer of a natural polymer with a synthetic polymer.

46. The method of any one of claims 10-45, wherein the one or more substrate(s) is a bead.

47. The method of claim 46, wherein the bead is a magnetic bead.

48. The method of any one of claims 10-45, wherein the one or more substrate(s) is a culture vessel.

49. The method of claim 48, wherein the culture vessel is a cell culture flask, cell culture bag, tube, plate, dish, or well.

50. The method of any one of claims 1-49, wherein the liquid culture medium comprises a chemically-defined liquid culture medium.

51. The method of any one of claims 1-49, wherein the liquid culture medium is selected from the group consisting of: AIM-V medium, NK MACS medium, EL837 medium, X-VIVO medium, X-VIVO 15 medium, X-VIVO 20 medium, LGM-3 lymphocyte growth medium, and LymphoONE T-cell expansion xeno-free medium.

52. The method of claim 51, wherein the liquid culture medium is AIM-V medium.

53. The method of any one of claims 1-49, 51, and 52, wherein the liquid culture medium comprises one or more of: serum, a serum replacement, penicillin, streptomycin, HEPES, and _{L}-alanyl-_{L}-glutamine.

54. The method of any one of claims 1-53, wherein the liquid culture medium further comprises one or both of (a) IL-2 or a functional fragment thereof and (b) IL-18 or a functional fragment thereof.

55. The method of claim 55, wherein the liquid culture medium comprises about U/mL to about 200 U/mL of IL-2 or a functional fragment thereof.

56. The method of claim 54 or 55, wherein the liquid culture medium comprises about U/mL to about 125 U/mL of IL-2 or a functional fragment thereof.

57. The method of any one of claims 54-56, wherein the liquid culture medium comprises about 0.1 nM to about 4.0 nM of IL-18 or a functional fragment thereof.

58. The method of any one of claims 54-57, wherein the liquid culture medium comprises about 0.2 nM to about 0.8 nM of IL-18 or a functional fragment thereof.

59. The method of any one of claims 1-58, wherein the liquid culture medium further comprises an anti-CD16 antibody or antigen-binding fragment thereof.

60. The method of any one of claims 1-59, wherein the liquid culture medium does not comprise IL-12 or a functional fragment thereof.

61. The method of any one of claims 1-60, wherein the liquid culture medium further comprises IL-21 or a functional fragment thereof.

62. The method of any one of claims 1-60, wherein the liquid culture medium does not comprise IL-21 or a functional fragment thereof.

63. The method of any one of claims 1-62, wherein the method comprises replenishing the liquid culture medium one or more times.

64. The method of claim 63, wherein the replenishing is performed using fed batch culturing.

65. The method of claim 63, wherein the replenishing is performed using perfusion culturing.

66. The method of claim 63 wherein the replenishing is performed by replacement of the liquid culture medium.

67. The method of any one of claims 63-66, wherein the replenishing of the liquid culture medium occurs about every one or two days.

68. The method of any one of claims 1-67, wherein the method further comprises, before the contacting step, culturing the NK cell in the presence of a feeder cell.

69. The method of any one of claims 1-68, wherein the method further comprises, after the contacting step, culturing the NK cell in the presence of a feeder cell.

70. The method of any one of claims 1-69, wherein the method further comprises, after the contacting step, culturing the NK cell in a liquid culture medium not comprising each of (i) a first polypeptide comprising B7-H6 or a functional fragment thereof, or an anti-NKp30 antigen-binding domain, (ii) a second polypeptide comprising 4-1BBL or a functional fragment thereof, and (iii) a third polypeptide comprising ICAM-1 or a functional fragment thereof.

71. The method of any one of claims 1-70, wherein the method further comprises, before the contacting step, introducing into the NK cell a nucleic acid encoding one or more exogenous polypeptides.

72. The method of any one of claims 1-71, wherein the method further comprises, after the contacting step, introducing into the NK cell a nucleic acid encoding one or more exogenous polypeptides.

73. The method of claim 71 or 72, wherein the one or more exogenous polypeptides is a chimeric antigen receptor or a therapeutic protein.

74. The method of any one of claims 1-73, wherein the NK cell is a genetically engineered NK cell.

75. The method of any one of claims 1-74, wherein the method results in about 8-fold to about 512,000-fold expansion of NK cells.

76. The method of any one of claims 1-75, wherein the method results in about 8-fold to about 80-fold expansion of NK cells at about day 10 after the contacting step.

77. The method of any one of claims 1-76, wherein the method results in about 64-fold to about 6,400-fold expansion of NK cells at about day 20 after the contacting step.

78. The method of any one of claims 1-77, wherein the method results in about 512-fold to about 512,000-fold expansion of NK cells at about day 30 after the contacting step.

79. A method of expanding an NK cell comprising:
contacting an NK cell disposed in a liquid culture medium, in the absence of a feeder cell, with (i) a first polypeptide comprising B7-H6 or a functional fragment thereof, or an anti-NKp30 antigen-binding domain, (ii) a second polypeptide comprising 4-1BBL or a functional fragment thereof, (iii) a third polypeptide comprising ICAM-1 or a functional fragment thereof, and (iv) a fourth polypeptide comprising IL-21 or a functional fragment thereof, under conditions sufficient for expansion of the NK cell.

80. The method of claim 79, wherein after the contacting step the method comprises periodically re-contacting the NK cell with the first polypeptide, the second polypeptide, the third polypeptide, and the fourth polypeptide.

81. The method of claim 80, wherein the method is performed over 30 days, and the re-contacting step is performed at about day 10 and at about day 20 after the contacting step.

82. The method of claim 79 or 80, wherein the method is performed over about 1 day to about 30 days.

83. The method of claim 82, wherein the method is performed over about 5 days to about days.

84. The method of claim 82, wherein the method is performed over about 10 days to about 30 days.

85. The method of any one of claims 79-84, wherein one or more of the first polypeptide, the second polypeptide, the third polypeptide, and the fourth polypeptide are present in the liquid culture medium.

86. The method of any one of claims 79-84, wherein two or more of the first polypeptide, the second polypeptide, the third polypeptide, and the fourth polypeptide are present in the liquid culture medium.

87. The method of any one of claims 79-84, wherein three or more of the first polypeptide, the second polypeptide, the third polypeptide, and the fourth polypeptide are present in the liquid culture medium.

88. The method of any one of claims 79-84, wherein the first polypeptide, the second polypeptide, the third polypeptide, and the fourth polypeptide are present in the liquid culture medium.

89. The method of any one of claims 79-84, wherein one or more of the first polypeptide, the second polypeptide, the third polypeptide, and the fourth polypeptide are directly or indirectly attached to one or more substrate(s).

90. The method of claim 79-89, wherein the first polypeptide, the second polypeptide, the third polypeptide, and the fourth polypeptide are present in approximately equal amounts or concentrations.

91. The method of claim 89 or 90, wherein one or more of the first polypeptide, the second polypeptide, the third polypeptide, and the fourth polypeptide are directly attached to the one or more substrate(s).

92. The method of claim 89 or 90, wherein one or more of the first polypeptide, the second polypeptide, the third polypeptide, and the fourth polypeptide are indirectly attached to the one or more substrate(s).

93. The method of any one of claims 89-92, wherein one or more of the first polypeptide, the second polypeptide, the third polypeptide, and the fourth polypeptide further comprises a tag.

94. The method of claim 93, wherein the one or more substrate(s) comprises an agent that specifically binds to the tag.

95. The method of claim 93 or 94, wherein the tag is an Fc domain, a myc tag, a HA tag, or a GST tag.

96. The method of claim 94 or 95, wherein: (a) the tag is an Fc domain, and the agent that specifically binds to the tag is an anti-Fc antibody or antigen-binding fragment thereof (b) the tag is a myc tag and the agent is an anti-myc tag antibody or antigen-binding fragment thereof (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

97. The method of any one of claims 89-96, wherein one or more of the first polypeptide, the second polypeptide, the third polypeptide, and the fourth polypeptide further comprises a linker.

98. The method of claim 97, wherein the linker comprises a glycine-serine (GS) repeat.

99. The method of claim 97 or 98, wherein the linker comprises an IgG hinge region.

100. The method of any one of claims 79-84, wherein two or more of the first polypeptide, the second polypeptide, the third polypeptide, and the fourth polypeptide are directly or indirectly attached to one or more substrate(s).

101. The method of claim 100, wherein two or more of the first polypeptide, the second polypeptide, the third polypeptide, and the fourth polypeptide are directly attached to the one or more substrate(s).

102. The method of claim 100, wherein two or more of the first polypeptide, the second polypeptide, the third polypeptide, and the fourth polypeptide are indirectly attached to the one or more substrate(s).

103. The method of any one of claims 100-102, wherein two or more of the first polypeptide, the second polypeptide, the third polypeptide, and the fourth polypeptide further comprise a tag.

104. The method of claim 103, wherein the one or more substrate(s) comprises an agent that specifically binds to the tag.

105. The method of claim 103 or 104, wherein the tag is an Fc domain, a myc tag, a HA tag, or a GST tag.

106. The method of claim 104 or 105, wherein: (a) the tag is an Fc domain, and the agent that specifically binds to the tag is an anti-Fc antibody or antigen-binding fragment thereof (b) the tag is a myc tag and the agent is an anti-myc tag antibody or antigen-binding fragment thereof (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

107. The method of any one of claims 100-107, wherein one or more of the first polypeptide, the second polypeptide, the third polypeptide, and the fourth polypeptide further comprises a linker.

108. The method of claim 107, wherein the linker comprises a glycine-serine (GS) repeat.

109. The method of claim 107 or 108, wherein the linker comprises an IgG hinge region.

110. The method of any one of claims 79-84, wherein three or more of the first polypeptide, the second polypeptide, the third polypeptide, and the fourth polypeptide are directly or indirectly attached to one or more substrate(s).

111. The method of claim 110, wherein three or more of the first polypeptide, the second polypeptide, the third polypeptide, and the fourth polypeptide are directly attached to the one or more substrate(s).

112. The method of claim 110, wherein three or more of the first polypeptide, the second polypeptide, the third polypeptide, and the fourth polypeptide are indirectly attached to the one or more substrate(s).

113. The method of any one of claims 110-112, wherein three or more of the first polypeptide, the second polypeptide, the third polypeptide, and the fourth polypeptide further comprise a tag.

114. The method of claim 113, wherein the one or more substrate(s) comprises an agent that specifically binds to the tag.

115. The method of claim 113 or 114, wherein the tag is an Fc domain, a myc tag, a HA tag, or a GST tag.

116. The method of claim 114 or 115, wherein: (a) the tag is an Fc domain, and the agent that specifically binds to the tag is an anti-Fc antibody or antigen-binding fragment thereof; (b) the tag is a myc tag and the agent is an anti-myc tag antibody or antigen-binding fragment thereof; (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

117. The method of any one of claims 110-116, wherein one or more of the first polypeptide, the second polypeptide, the third polypeptide, and the fourth polypeptide further comprises a linker.

118. The method of claim 117, wherein the linker comprises a glycine-serine (GS) repeat.

119. The method of claim 117 or 118, wherein the linker comprises an IgG hinge region.

120. The method of any one of claims 79-84, wherein the first polypeptide, the second polypeptide, the third polypeptide, and the fourth polypeptide are directly or indirectly attached to one or more substrate(s).

121. The method of claim 120, wherein the first polypeptide, the second polypeptide, the third polypeptide, and the fourth polypeptide are directly attached to the one or more substrate(s).

122. The method of claim 120, wherein the first polypeptide, the second polypeptide, the third polypeptide, and the fourth polypeptide are indirectly attached to the one or more substrate(s).

123. The method of any one claims 120-122, wherein the first polypeptide, the second polypeptide, the third polypeptide, and the fourth polypeptide further comprise a tag.

124. The method of claim 123, wherein the one or more substrate(s) comprises an agent that specifically binds to the tag.

125. The method of claim 123 or 124, wherein the tag is an Fc domain, a myc tag, a HA tag, or a GST tag.

126. The method of claim 124 or 125, wherein: (a) the tag is an Fc domain, and the agent that specifically binds to the tag is an anti-Fc antibody or antigen-binding fragment thereof (b) the tag is a myc tag and the agent is an anti-myc tag antibody or antigen-binding fragment thereof (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

127. The method of any one of claims 120-126, wherein one or more of the first polypeptide, the second polypeptide, the third polypeptide, and the fourth polypeptide further comprises a linker.

128. The method of claim 127, wherein the linker comprises a glycine-serine (GS) repeat.

129. The method of claim 127 or 128, wherein the linker comprises an IgG hinge region.

130. The method of claim 79-129, wherein the substrate comprises a hydrogel.

131. The method of claim 79-130, wherein the substrate comprises a natural polymer, a synthetic polymer, or a co-polymer.

132. The method of claim 131, wherein the natural polymer is selected from the group consisting of: alginate, agarose, carrageenan, chitosan, dextran, carboxymethylcellulose, heparin, hyaluronic acid, polyamino acids, collagen, gelatin, fibrin, fibrous protein-based biopolymers, and any combination thereof.

133. The method of claim 131, wherein the synthetic polymer is selected from the group consisting of: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof.

134. The method of claim 131, wherein the co-polymer is a copolymer of natural polymers, a copolymer of synthetic polymers or a copolymer of a natural polymer with a synthetic polymer.

135. The method of any one of claims 79-134, wherein the one or more substrate(s) is a bead.

136. The method of claim 134, wherein the bead is a magnetic bead.

137. The method of any one of claims 79-134, wherein the one or more substrate(s) is a culture vessel.

138. The method of claim 137, wherein the culture vessel is a cell culture flask, cell culture bag, tube, plate, dish, or well.

139. The method of any one of claims 79-138, wherein the liquid culture medium is a chemically-defined liquid culture medium.

140. The method of any one of claims 79-138, wherein the liquid culture medium is selected from the group consisting of: AIM-V medium, NK MACS medium, EL837 medium, X-VIVO 10 medium, X-VIVO 15 medium, X-VIVO 20 medium, LGM-3 lymphocyte growth medium, and LYMPHOONE T-cell expansion xeno-free medium.

141. The method of claim 140, wherein the liquid culture medium is AIM-V medium.

142. The method of any one of claims 79-138, 140, and 141, wherein the liquid culture medium comprises one or more of: serum, a serum replacement, penicillin, streptomycin, HEPES, and _{L}-alanyl-_{L}-glutamine.

143. The method of any one of claims 79-142, wherein the liquid culture medium further comprises one or both of (a) IL-2 or a functional fragment thereof and (b) IL-18 or a functional fragment thereof.

144. The method of claim 143, wherein the liquid culture medium comprises about U/mL to about 200 U/mL of IL-2 or a functional fragment thereof.

145. The method of claim 143 or 144, wherein the liquid culture medium comprises about 75 U/mL to about 125 U/mL of IL-2 or a functional fragment thereof.

146. The method of any one of claims 143-145, wherein the liquid culture medium comprises about 0.1 nM to about 4.0 nM of IL-18 or a functional fragment thereof.

147. The method of claim 146, wherein the liquid culture medium comprises about nM to about 0.8 nM of IL-18 or a functional fragment thereof.

148. The method of any one of claims 79-147, wherein the liquid culture medium further comprises an anti-CD16 antibody or antigen-binding fragment thereof.

149. The method of any one of claims 79-148, wherein the liquid culture medium does not comprise IL-12 or a functional fragment thereof.

150. The method of any one of claims 79-149, wherein the method comprises replenishing the liquid culture medium one or more times.

151. The method of claim 150, wherein the replenishing is performed using fed batch culturing.

152. The method of claim 150, wherein the replenishing is performed using perfusion culturing.

153. The method of claim 150, wherein the replenishing is performed by replacement of the liquid culture medium.

154. The method of any one of claims 150-154, wherein the replenishing of the liquid culture medium occurs about every one or two days.

155. The method of any one of claims 79-154, wherein the method further comprises, before the contacting step, culturing the NK cell in the presence of a feeder cell.

156. The method of any one of claims 79-155, wherein the method further comprises, after the contacting step, culturing the NK cell in the presence of a feeder cell.

157. The method of any one of claims 79-156, wherein the method further comprises, after the contacting step, culturing the NK cell in a liquid culture medium not comprising each of (i) a first polypeptide comprising B7-H6 or a functional fragment thereof, or an anti-NKp30 antigen-binding domain, (ii) a second polypeptide comprising 4-1BBL or a functional fragment thereof, (iii) a third polypeptide comprising ICAM-1 or a functional fragment thereof; and (iv) a fourth polypeptide comprising IL-21 or a functional fragment thereof.

158. The method of any one of claims 79-157, wherein the method further comprises, before the contacting step, introducing into the NK cell a nucleic acid encoding one or more exogenous polypeptides.

159. The method of any one of claims 79-157, wherein the method further comprises, after the contacting step, introducing into the NK cell a nucleic acid encoding one or more exogenous polypeptides.

160. The method of claim 158 or 159, wherein the one or more exogenous polypeptides is a chimeric antigen receptor or a therapeutic protein.

161. The method of any one of claims 79-160, wherein the NK cell is a genetically engineered NK cell.

162. The method of any one of claims 79-161, wherein the method results in about 8-fold to about 512,000-fold expansion of NK cells.

163. The method of any one of claims 79-162, wherein the method results in about 8-fold to about 80,000-fold expansion of NK cells at about day 10 after the contacting step.

164. The method of any one of claims 79-163, wherein the method results in about 64-fold to about 6,400-fold expansion of NK cells at about day 20 after the contacting step.

165. The method of any one of claims 79-164, wherein the method results in about 512-fold to about 512,000-fold expansion of NK cells at about day 30 after the contacting step.

166. An NK cell produced by the method of any one of claims 1-165.

167. A pharmaceutical composition comprising the NK cell of claim 166.

168. A method of treating a subject in need thereof comprising administering to the subject an NK cell of claim 166 or the pharmaceutical composition of claim 167.

169. A kit comprising:
one or more substrate(s) that together comprise: (i) a first polypeptide comprising B7-H6 or a functional fragment thereof, or an anti-NKp30 antigen-binding domain, (ii) a second polypeptide comprising 4-1BBL or a functional fragment thereof, and (iii) a third polypeptide comprising ICAM-1 or a functional fragment thereof, directly or indirectly attached to the one or more substrate(s).

170. The kit of claim 169, wherein the first polypeptide, the second polypeptide, and the third polypeptide are directly attached to the one or more substrate(s).

171. The kit of claim 169, wherein the first polypeptide, the second polypeptide, and the third polypeptide are indirectly attached to the one or more substrate(s).

172. The kit of any one of claims 169-171, wherein the first polypeptide, the second polypeptide, and the third polypeptide further comprise a tag.

173. The kit of claim 172, wherein the one or more substrate(s) comprises an agent that specifically binds to the tag.

174. The kit of claim 172 or 173, wherein the tag is an Fc domain, a myc tag, a HA tag, or a GST tag.

175. The kit of claim 173 or 174, wherein: (a) the tag is an Fc domain, and the agent that specifically binds to the tag is an anti-Fc antibody or antigen-binding fragment thereof; (b) the tag is a myc tag and the agent is an anti-myc tag antibody or antigen-binding fragment thereof; (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

176. The kit of any one of claims 169-175, wherein one or more of the first polypeptide, the second polypeptide, and the third polypeptide further comprises a linker.

177. The kit of claim 176, wherein the linker comprises a glycine-serine (GS) repeat.

178. The kit of claim 176 or 177, wherein the linker comprises an IgG hinge region.

179. The kit of any one of claims 169-178, wherein the first polypeptide, the second polypeptide, and the third polypeptide are present in approximately equal amounts or concentrations.

180. The kit of any one claims 169-179, wherein the one or more substrate(s) comprises a hydrogel.

181. The kit of any one of claims 169-180, wherein the one or more substrate(s) comprises a natural polymer, a synthetic polymer, or a co-polymer.

182. The kit of claim 181, wherein the natural polymer is selected from the group consisting of: alginate, agarose, carrageenan, chitosan, dextran, carboxymethylcellulose, heparin, hyaluronic acid, polyamino acids, collagen, gelatin, fibrin, fibrous protein-based biopolymers, and any combination thereof.

183. The kit of claim 181, wherein the synthetic polymer is selected from the group consisting of: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof.

184. The kit of claim 181, wherein the co-polymer is a copolymer of natural polymers, a copolymer of synthetic polymers or a copolymer of a natural polymer with a synthetic polymer.

185. The kit of any one of claims 169-184, wherein the one or more substrate(s) is a bead.

186. The kit of claim 185, wherein the bead is a magnetic bead.

187. The kit of any one of claims 169-184, wherein the one or more substrate(s) is a culture vessel.

188. The kit of claim 187, wherein the culture vessel is a cell culture flask, cell culture bag, tube, plate, dish, or well.

189. The kit of any one of claims 169-188, wherein the kit further comprises a liquid culture medium.

190. The kit of claim 189, wherein the liquid culture medium is a chemically-defined liquid culture medium.

191. The kit of claim 189, wherein the liquid culture medium is selected from the group consisting of: AIM-V medium, NK MACS medium, EL837 medium, X-VIVO 10 medium, X-VIVO 15 medium, X-VIVO 20 medium, LGM-3 lymphocyte growth medium, and LymphoONE T-cell expansion xeno-free medium.

192. The kit of claim 191, wherein the liquid culture medium is AIM-V medium.

193. The kit of any one of claims 189, 191, and 192, wherein the liquid culture medium comprises one or more of: serum, a serum replacement, penicillin, streptomycin, HEPES, and _{L}-alanyl-_{L}-glutamine.

194. The kit of any one of claims 189-193, wherein the liquid culture medium further comprises one or both of (a) IL-2 or a functional fragment thereof and (b) IL-18 or a functional fragment thereof.

195. The kit of claim 194, wherein the liquid culture medium comprises about 50 U/mL to about 200 U/mL of IL-2 or a functional fragment thereof.

196. The kit of claim 194 or 195, wherein the liquid culture medium comprises about U/mL to about 125 U/mL of IL-2 or a functional fragment thereof.

197. The kit of any one of claims 194-196, wherein the liquid culture medium comprises about 0.1 nM to about 4.0 nM of IL-18 or a functional fragment thereof.

198. The kit of any one of claims 194-197, wherein the liquid culture medium comprises about 0.2 nM to about 0.8 nM of IL-18 or a functional fragment thereof.

199. The kit of any one of claims 189-198, wherein the liquid culture medium further comprises an anti-CD16 antibody or antigen-binding fragment thereof.

200. The kit of any one of claims 189-199, wherein the liquid culture medium does not comprise IL-12 or a functional fragment thereof.

201. The kit of any one of claims 189-200, wherein the liquid culture medium further comprises IL-21 or a functional fragment thereof.

202. The kit of any one of claims 189-200, wherein the liquid culture medium does not comprise IL-21 or a functional fragment thereof.

203. A kit comprising:
one or more substrate(s) that together comprise: (i) a first polypeptide comprising B7-H6 or a functional fragment thereof, or an anti-NKp30 antigen-binding domain, (ii) a second polypeptide comprising 4-1BBL or a functional fragment thereof, (iii) a third polypeptide comprising ICAM-1 or a functional fragment thereof, and (iv) a fourth polypeptide comprising IL-21 or a functional fragment thereof, directly or indirectly attached to the one or more substrate(s).

204. The kit of claim 203, wherein the first polypeptide, the second polypeptide, the third polypeptide, and the fourth polypeptide are directly attached to the one or more substrate(s).

205. The kit of claim 203, wherein the first polypeptide, the second polypeptide, the third polypeptide, and the fourth polypeptide are indirectly attached to the one or more substrate(s).

206. The kit of any one of claims 203-204, wherein the first polypeptide, the second polypeptide, the third polypeptide, and the fourth polypeptide further comprise a tag.

207. The kit of claim 206, wherein the one or more substrate(s) comprise(s) an agent that specifically binds to the tag.

208. The kit of claim 206 or 207, wherein the tag is an Fc domain, a myc tag, a HA tag, or a GST tag.

209. The kit of claim 207 or 208, wherein: (a) the tag is an Fc domain, and the agent that specifically binds to the tag is an anti-Fc antibody or antigen-binding fragment thereof; (b) the tag is a myc tag and the agent is an anti-myc tag antibody or antigen-binding fragment thereof; (c) the tag is an HA tag and the agent that specifically binds to the tag is an anti-HA tag antibody or antigen-binding fragment thereof; or (d) the tag is a GST tag and the agent that specifically binds to the tag is an anti-GST tag antibody or antigen-binding fragment thereof.

210. The kit of any one of claims 203-29, wherein one or more of the first polypeptide, the second polypeptide, the third polypeptide, and the fourth polypeptide further comprises a linker.

211. The kit of claim 210, wherein the linker comprises a glycine-serine (GS) repeat.

212. The kit of claim 210 or 211, wherein the linker comprises an IgG hinge region.

213. The kit of any one of claims 203-212, wherein the first polypeptide, the second polypeptide, the third polypeptide, and the fourth polypeptide are present in approximately equal amounts or concentrations.

214. The kit of any one of claims 203-213, wherein the one or more substrate(s) comprises a hydrogel.

215. The kit of any one of claims 203-214, wherein the one or more substrate(s) comprises a natural polymer, a synthetic polymer, or a co-polymer.

216. The kit of claim 215, wherein the natural polymer is selected from the group consisting of: alginate, agarose, carrageenan, chitosan, dextran, carboxymethylcellulose, heparin, hyaluronic acid, polyamino acids, collagen, gelatin, fibrin, fibrous protein-based biopolymers, and any combination thereof.

217. The kit of claim 215, wherein the synthetic polymer is selected from the group consisting of: alginic acid-polyethylene glycol copolymer, poly(ethylene glycol) (PEG), poly(2-methyl-2-oxazoline) (PMOXA), poly(ethylene oxide), poly(vinyl alcohol), and poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ehtylhexyl)phthalate, di-iso-nonyl-1,2-cyclohexanedicarboxylate, polytetrafluoroethylene, ethylene vinyl alcohol copolymer, poly(hexamethylene diisocyanate), poly(ethylene) (e.g., high density, low density, or ultrahigh molecular weight poly(ethylene), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), and any combination thereof.

218. The kit of claim 215, wherein the co-polymer is a copolymer of natural polymers, a copolymer of synthetic polymers or a copolymer of a natural polymer with a synthetic polymer.

219. The kit of any one of claims 203-218, wherein the one or more substrate(s) is a bead.

220. The kit of claim 219, wherein the bead is a magnetic bead.

221. The kit of any one of claims 203-218, wherein the one or more substrate(s) is a culture vessel.

222. The kit of claim 221, wherein the culture vessel is a cell culture flask, cell culture bag, tube, plate, dish, or well.

223. The kit of any one of claims 203-222, wherein the kit further comprises a liquid culture medium.

224. The kit of claim 223, wherein the liquid culture medium is a chemically-defined liquid culture medium.

225. The kit of claim 223, wherein the liquid culture medium is selected from the group consisting of: AIM-V medium, NK MACS medium, EL837 medium, X-VIVO 10 medium, X-VIVO 15 medium, X-VIVO 20 medium, LGM-3 lymphocyte growth medium, and LYMPHOONE T-cell expansion xeno-free medium.

226. The kit of claim 225, wherein the liquid culture medium is AIM-V medium.

227. The kit of any one of claims 223, 224, and 226, wherein the liquid culture medium comprises one or more of: serum, a serum replacement, penicillin, streptomycin, HEPES, and _{L}-alanyl-_{L}-glutamine.

228. The kit of any one of claims 223-227, wherein the liquid culture medium further comprises one or both of (a) IL-2 or a functional fragment thereof and (b) IL-18 or a functional fragment thereof.

229. The kit of claim 228, wherein the liquid culture medium comprises about 50 U/mL to about 200 U/mL of IL-2 or a functional fragment thereof.

230. The kit of claim 228 or 229, wherein the liquid culture medium comprises about 75 U/mL to about 125 U/mL of IL-2 or a functional fragment thereof.

231. The kit of any one of claims 228-230, wherein the liquid culture medium comprises about 0.1 nM to about 4.0 nM of IL-18 or a functional fragment thereof.

232. The kit of any one of claims 228-231, wherein the liquid culture medium comprises about 0.2 nM to about 0.8 nM of IL-18 or a functional fragment thereof.

233. The kit of any one of claims 223-232, wherein the liquid culture medium further comprises an anti-CD16 antibody or antigen-binding fragment thereof.

234. The kit of any one of claims 223-233, wherein the liquid culture medium does not comprise IL-12 or a functional fragment thereof.


SEQUENCE LISTING (29 sequences)

SEQ ID NO: 1 - protein, 430 aa, Homo sapiens
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 2 - protein, 238 aa, Homo sapiens
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 3 - protein, 300 aa, Rattus sp.
LPSAGGLELEMAGTTQIVFLHEDVTIPCKILGSLHLDLSIVGVIWSLKKDGDESEVFKFYGDQLEAVRPGANVSLLGLEHGDASLYLPRFELWEAGEYQCKVVVTPEKKEGTTRLEVVAHPNMSLSEKPATARGGKEKLIICQLDGFYPEALDIKWMGSALKDSHFQEITEGVVTGPTVKNDDGTFSVTSSLALKPALEDHMYQCVVWHRSWLMPQSLNVTVFENTRDSTHGTVPPTAEVGPPVSEPRSVMIYVTIIGLCILLFSVIVCGLWKWKRLTTSNTGLCLRLDVRCCLPSFRNT

SEQ ID NO: 4 - protein, 248 aa, Rattus sp.
LPSAGGLELEMAGTTQIVFLHEDVTIPCKILGSLHLDLSIVGVIWSLKKDGDESEVFKFYGDQLEAVRPGANVSLLGLEHGDASLYLPRFELWEAGEYQCKVVVTPEKKEGTTRLEVVAHPNMSLSEKPATARGGKEKLIICQLDGFYPEALDIKWMGSALKDSHFQEITEGVVTGPTVKNDDGTFSVTSSLALKPALEDHMYQCVVWHRSWLMPQSLNVTVFENTRDSTHGTVPPTAEVGPPVSEPR

SEQ ID NO: 5 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 204 aa, Homo sapiens
CPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 7 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 8 - protein, 206 aa, Mus musculus
RTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 9 - protein, 308 aa, Rattus sp.
MDQHALDVGGTVDAGHPPGTAQPPDAAFLRDTGLPSDAAFLVDTVRLTEAALPTDAACPAVNVRDPEAAWPPALNFCSRHPRLYCLGGLVLLLLVACVPIFIRILPQPALTITTSPDLDTGENTADQVTPVSHIGCANTTQQGFPVFAKLLAKNQASLSNTTLNWHSQEGAGSSYLSRGLRYEEDKKELVVDSPGLYYVFLELKLSPTSINTGRKVQGWVSLVLQAKPQVDHLDSLALTVELFPCSMEDKLVDRSWNRLLRLKAGHRLSVDLRAYLHGAQDAYRDWELSHANTTSFGLFLVKPDPPQG

SEQ ID NO: 10 - protein, 206 aa, Rattus sp.
RILPQPALTITTSPDLDTGENTADQVTPVSHIGCANTTQQGFPVFAKLLAKNQASLSNTTLNWHSQEGAGSSYLSRGLRYEEDKKELVVDSPGLYYVFLELKLSPTSINTGRKVQGWVSLVLQAKPQVDHLDSLALTVELFPCSMEDKLVDRSWNRLLRLKAGHRLSVDLRAYLHGAQDAYRDWELSHANTTSFGLFLVKPDPPQG

SEQ ID NO: 11 - protein, 505 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 12 - protein, 403 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELK

SEQ ID NO: 13 - protein, 510 aa, Mus musculus
QVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 14 - protein, 458 aa, Mus musculus
QVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNN

SEQ ID NO: 15 - protein, 518 aa, Rattus sp.
QVSIHPTEAFLPRGGSVQVNCSSSCEDENLGLGLETNWMKDELSSGHNWKLFKLSDIGEDSRPLCFENCGTTQSSASATITVYSFPERVELDPLPAWQQVGKNLILRCLVEGGAPRTQLSVVLLRGNETLSRQAVDGDPKEITFTVLASRGDHGANFSCFTELDLRPQGLSLFKNVSEVRQLRTFDLPTRVLKLDTPDLLEVGTQQKFLCSLEGLFPASEAQIYLEMGGQMLTLESTNSRDFVSATASVEVTEKLDRTLQLRCVLELADQTLEMEKTLRIYNFSAPILTLSQPEVSEGDQVTVKCEAHGGAQVVLLNSTSPRPPTSQGTSPRPPTSQIQFTLNASPEDHKRRFFCSAALEVDGKSLFKNQTLELHVLYGPHLDKKDCLGNWTWQEGSQQTLTCQPQGNPAPNLTCSRKADGVPLPIGMVKSVKREMNGTYKCRAFSSRGSITRDVHLTVLYHDQNTWVIIVGVLVLIIAGFVIVASIYTYYRQRKIRIYKLQKAQEEALKLKVQAPPP

SEQ ID NO: 16 - protein, 465 aa, Rattus sp.
QVSIHPTEAFLPRGGSVQVNCSSSCEDENLGLGLETNWMKDELSSGHNWKLFKLSDIGEDSRPLCFENCGTTQSSASATITVYSFPERVELDPLPAWQQVGKNLILRCLVEGGAPRTQLSVVLLRGNETLSRQAVDGDPKEITFTVLASRGDHGANFSCFTELDLRPQGLSLFKNVSEVRQLRTFDLPTRVLKLDTPDLLEVGTQQKFLCSLEGLFPASEAQIYLEMGGQMLTLESTNSRDFVSATASVEVTEKLDRTLQLRCVLELADQTLEMEKTLRIYNFSAPILTLSQPEVSEGDQVTVKCEAHGGAQVVLLNSTSPRPPTSQGTSPRPPTSQIQFTLNASPEDHKRRFFCSAALEVDGKSLFKNQTLELHVLYGPHLDKKDCLGNWTWQEGSQQTLTCQPQGNPAPNLTCSRKADGVPLPIGMVKSVKREMNGTYKCRAFSSRGSITRDVHLTVLYHDQN

SEQ ID NO: 17 - protein, 133 aa, Homo sapiens [sequence omitted]

SEQ ID NO: 18 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 19 - protein, 135 aa, Rattus sp.
APTSSPAKETQQHLEQLLLDLQVLLRGIDNYKNLKLPMMLTFKFYLPKQATELKHLQCLENELGALQRVLDLTQSKSFHLEDAGNFISNIRVTVVKLKGSENKFECQFDDEPATVVEFLRRWIAICQSIISTMTQ

SEQ ID NO: 20 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 21 - protein, 157 aa, Mus musculus
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 22 - protein, 158 aa, Rattus sp.
HFGRLHCTTAVIRSINDQVLFVDKRNPPVFEDMPDIDRTANESQTRLIIYMYKDSEVRGLAVTLSVKDGRMSTLSCKNKIISFEEMNPPENIDDIKSDLIFFQKRVPGHNKMEFESSLYEGHFLACQKEDDAFKLVLKRKDENGDKSVMFTLTNLHQS

SEQ ID NO: 23 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 24 - protein, 129 aa, Mus musculus
HKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 25 - protein, 129 aa, Rattus sp.
HKSSPQRPDHLLIRLRHLMDIVEQLKIYENDLDPELLTAPQDVKGQCEHEAFACFQKAKLKPSNTGNNKTFINDLLAQLRRRLPAKRTGNKQRHMAKCPSCDLYEKKTPKEFLERLKWLLQKMIHQHLS

SEQ ID NO: 26 - protein, 120 aa, Homo sapiens
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPA

SEQ ID NO: 27 - protein, 112 aa, Homo sapiens
KVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLE

SEQ ID NO: 28 - protein, 112 aa, Rattus sp.
GGLELEMAGTTQIVFLHEDVTIPCKILGSLHLDLSIVGVIWSLKKDGDESEVFKFYGDQLEAVRPGANVSLLGLEHGDASLYLPRFELWEAGEYQCKVVVTPEKKEGTTRLE

SEQ ID NO: 29 - protein, 63 aa, Rattus sp.
GGSVQVNCSSSCEDENLGLGLETNWMKDELSSGHNWKLFKLSDIGEDSRPLCFENCGTTQSSA